(19) United States
(12) Patent Application Publication
(10) Pub. No.: US 2022/0340566 A1
(43) Pub. Date: Oct. 27, 2022

(54) BENZYL AMINE-CONTAINING 5,6-HETEROAROMATIC COMPOUNDS USEFUL AGAINST MYCOBACTERIAL INFECTION

(71) Applicants: Shionogi & Co., Ltd., Osaka (JP); HSIRI THERAPEUTICS, INC., Media, PA (US)

(72) Inventors: Manabu FUJITANI, Osaka (JP); Tsutomu IWAKI, Osaka (JP); Rina NAKAMURA, Osaka (JP); Marvin J. MILLER, South Bend, IN (US); Garrett C. MORASKI, Bozeman, MT (US)

(73) Assignees: Shionogi & Co., Ltd., Osaka (JP); HSIRI THERAPEUTICS, INC., Media, PA (US)

(21) Appl. No.: 17/641,668

(22) PCT Filed: Sep. 10, 2020
(86) PCT No.: PCT/US2020/050173

§ 371 Date: Mar. 9, 2022

Related U.S. Application Data
(60) Provisional application No. 62/898,066, filed on Sep. 10, 2019.

(51) Int. Cl. C07D 471/04; A61P 31/10

(52) U.S. Cl. CPC C07D 471/04; A61P 31/10

(57) ABSTRACT

A compound represented by formula (I):

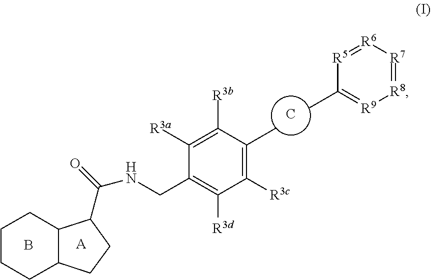

or its pharmaceutically acceptable salt,
wherein
a group represented by formula:

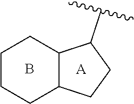

is a group represented by formula:

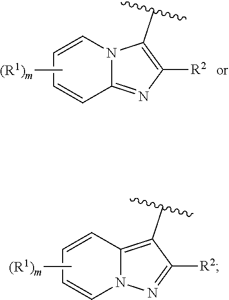

R¹ is each independently halogen, hydroxy, cyano or the like;
m is 0, 1, 2, 3 or 4;
R² is a hydrogen atom, halogen, cyano, substituted or unsubstituted alkyl or the like;
R^{3a}, R^{3b}, R^{3c} and R^{3d} are each independently hydrogen atom, halogen or the like, with the proviso that R^{3a}, R^{3b}, R^{3c} and R^{3d} are not simultaneously hydrogen atom; ring C is represented as follows:

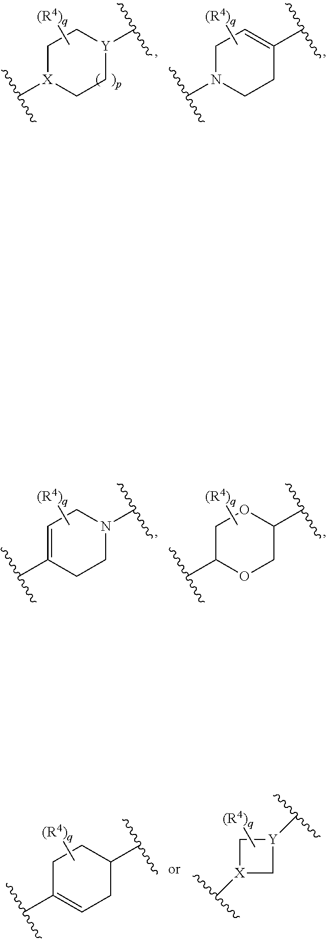

X is CH or N;
Y is CH or N;
R⁴ is each independently halogen, hydroxy, cyano or the like;
p is 0 or 1;
q is 0, 1, 2, 3 or 4;
R⁵ is CR^{5C} or N; R⁶ is CR^{6C} or N; R⁷ is CR^{7C} or N; R⁸ is CR^{8C} or N; R⁹ is CR^{9C} or N; with the proviso that R⁵, R⁶, R⁷, R⁸ and R⁹ are not simultaneously N;
R^{5C}, R^{6C}, R^{7C}, R^{8C} and R^{9C} are each independently hydrogen atom or the like.

### RELATED APPLICATIONS

This application claims the benefit of U.S. Provisional Application No. 62/898,066, filed Sep. 10, 2019, the entire contents of which are hereby incorporated by reference.

### TECHNICAL FIELD

The present invention relates to novel compounds. The invention also relates to such compounds for use as a pharmaceutical composition and further for the use in the treatment of bacterial diseases, including diseases caused by pathogenic mycobacteria such as non-tuberculosis mycobacteria. Such compounds may work by interfering with synthesis of ATP in pathogenic mycobacteria, with the inhibition of cytochrome bc1 activity as the primary mode of action.

### BACKGROUND

Genus Mycobacterium has 95 well characterized species. Over the centuries, two well known mycobacterial species, namely, Mycobacterium tuberculosis and M. Leprae have been the known causes of immense human suffering. Most other mycobacteria are present in the environment and their pathogenic potential has been recognized since the beginning of the last century. These mycobacteria are called non-tuberculous mycobacteria (NTM). Whereas the incidence of tuberculosis (TB) is decreasing, a new health concern has been raised globally by NTM. Pulmonary disease caused by NTM is characterized by progressive, irreversible pulmonary damage and increased mortality. About 80% of pulmonary NTM disease is caused by Mycobacterium avium complex (MAC: M. avium, M. intracellulare and M. chimaera).

The annual prevalence of NTM pulmonary disease varies in different regions, ranging from 0.2/100,000 to 14.7/100,000 with an overall alarming growth rate. The disease is more prevalent after age 60 where the estimated prevalence is from 19.6/100,000 during 1994-1996 to 26.7/100,000 during 2004-2006 in the US.

Different from TB, NTM are opportunistic pathogens, causing mostly TB-like pulmonary diseases in immunocompromised patients or patients with pre-existing lung conditions, such as cystic fibrosis (CF), bronchiectasis or chronic obstructive pulmonary disease (COPD). In addition, post-menopausal women without pre-existing structural pulmonary disease represent another risk group for NTM lung disease. These women, primarily older women of Caucasian or Asian descent, present with nodular bronchiectasis as their NTM lung disease.

Described herein are agents that can be a treatment for one or both of TB and NTM infections. There is an unmet medical need for compounds effective against one or both of TB and NTM pathogens, and the present compounds are considered to be effective against the drug resistant strains as a single agent or in combination therapy.

Patent Document 1 discloses a variety of compounds having a cytochrome bc1 inhibitory activity. For example, the following compounds are disclosed.

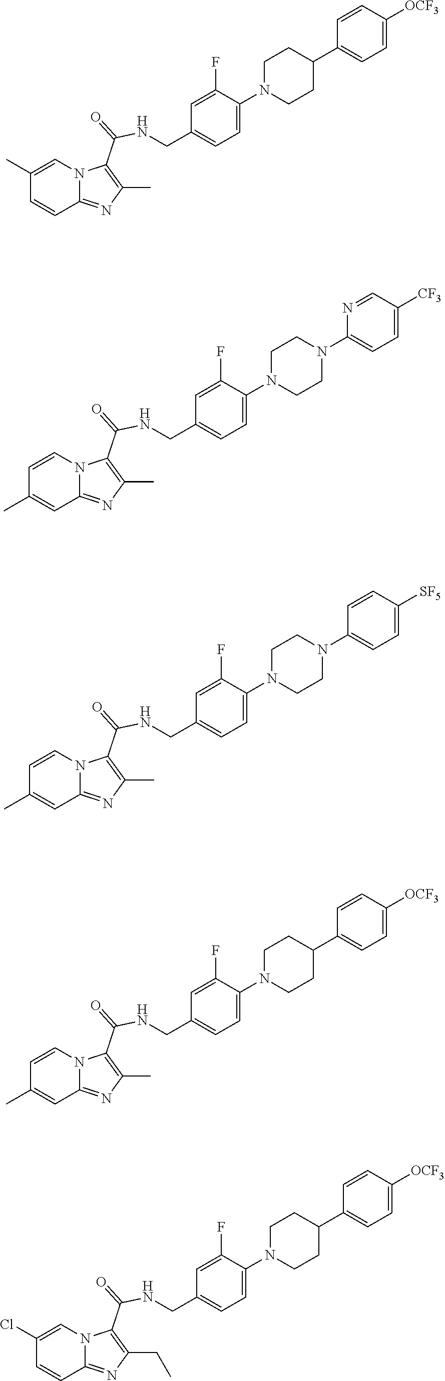

Patent Documents 2 to 12 disclose a variety of compounds having cytochrome bc1 inhibitory activity. For example, the following compound is disclosed in Patent Document 12.

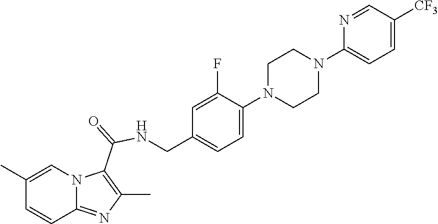 [SMILES: Cc1ccc2nc(C)c(C(=O)NCc3ccc(N4CCN(c5ccc(C(F)(F)F)cn5)CC4)c(F)c3)n2c1]

### PATENT DOCUMENTS

Patent Document 1: WO2017/049321
Patent Document 2: WO2011/057145
Patent Document 3: WO2014/015167
Patent Document 4: WO2011/113606
Patent Document 5: WO2015/014993
Patent Document 6: WO2017/001660
Patent Document 7: WO2017/001661
Patent Document 8: WO2017/216281
Patent Document 9: WO2017/216283
Patent Document 10: WO2018/158280
Patent Document 11: US2017/0313697
Patent Document 12: WO2019/175737

### DISCLOSURE OF INVENTION

### Problems to be Solved by the Invention

An object of the present invention is to provide a compound useful for treating or preventing bacterial diseases, including diseases caused by pathogenic mycobacteria such as non-tuberculosis mycobacteria, or its pharmaceutically acceptable salt, and a pharmaceutical composition containing thereof.

### Means for Solving the Problems

As a result of intensive studies in order to solve the above problems, the present inventors succeeded in synthesizing an excellent compound for the prevention and/or treatment of a mycobacterial infection, especially non-tuberculous mycobacterial infection.

### BRIEF DESCRIPTION OF SEVERAL EMBODIMENTS OF THE INVENTION

(1) A compound represented by formula (I):

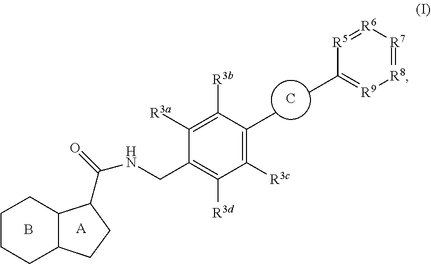

or its pharmaceutically acceptable salt,
wherein
a group represented by formula:

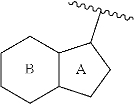

is a group represented by formula:

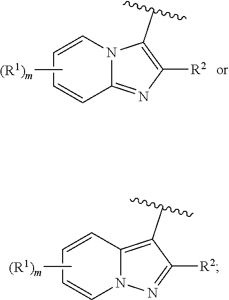

R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted alkylsulfanyl, substituted or unsubstituted alkenylsulfanyl, substituted or unsubstituted alkynylsulfanyl, substituted or unsubstituted alkylsulfinyl, substituted or unsubstituted alkenylsulfinyl, substituted or unsubstituted alkynylsulfinyl, substituted or unsubstituted alkylsulfonyl, substituted or unsubstituted alkenylsulfonyl or substituted or unsubstituted alkynylsulfonyl;
m is 0, 1, 2, 3 or 4;
R² is a hydrogen atom, halogen, cyano, substituted or unsubstituted alkyl, substituted or unsubstituted alkenyl or substituted or unsubstituted alkynyl;
R^{3a}, R^{3b}, R^{3c} and R^{3d} are each independently hydrogen atom, halogen, hydroxy, cyano, substituted or unsubstituted alkyl, substituted or unsubstituted alkenyl or substituted or unsubstituted alkynyl, with the proviso that R^{3a}, R^{3b}, R^{3c} and R^{3d} are not simultaneously hydrogen atom;
ring C is represented as follows:

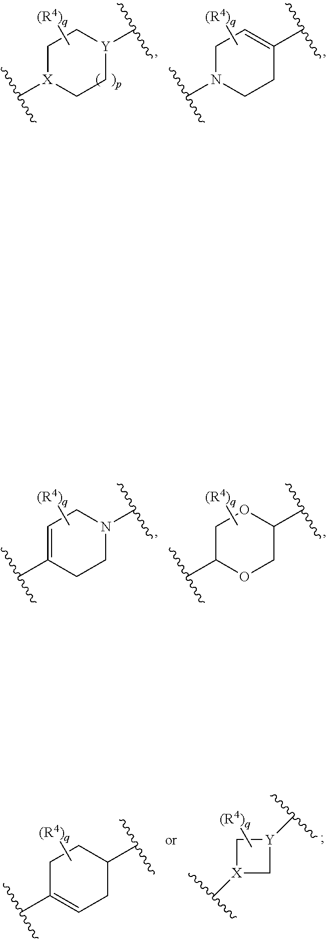

### X is CH or N;

### Y is CH or N;

R⁴ is each independently halogen, hydroxy, cyano, substituted or unsubstituted alkyl, substituted or unsubstituted alkenyl or substituted or unsubstituted alkynyl; two R⁴ groups attached to adjacent carbon atoms may be taken together with the carbon atoms to which they are attached to form a substituted or unsubstituted 5- to 6-membered non-aromatic carbocycle or a substituted or unsubstituted 5- to 6-membered non-aromatic heterocycle;
two R⁴ groups attached to a same carbon atom may be taken together with the carbon atom to which they are attached to form a substituted or unsubstituted 3- to 6-membered non-aromatic carbocycle or a substituted or unsubstituted 3- to 6-membered non-aromatic heterocycle;
two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano or substituted or unsubstituted alkyl;
R^{4N} is each independently a hydrogen atom or substituted or unsubstituted alkyl;
p is 0 or 1;
q is 0, 1, 2, 3 or 4;
R⁵ is CR^{5C} or N;
R⁶ is CR^{6C} or N;
R⁷ is CR^{7C} or N;
R⁸ is CR^{8C} or N;
R⁹ is CR^{9C} or N;
with the proviso that R⁵, R⁶, R⁷, R⁸ and R⁹ are not simultaneously N; R^{5C}, R^{6C}, R^{7C}, R^{8C} and R^{9C} are each independently hydrogen atom, halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted aromatic carbocyclyloxy, substituted or unsubstituted non-aromatic carbocyclyloxy, substituted or unsubstituted aromatic heterocyclyloxy, substituted or unsubstituted non-aromatic heterocyclyloxy or pentafluorothio;
with the proviso that compounds shown below are excluded:

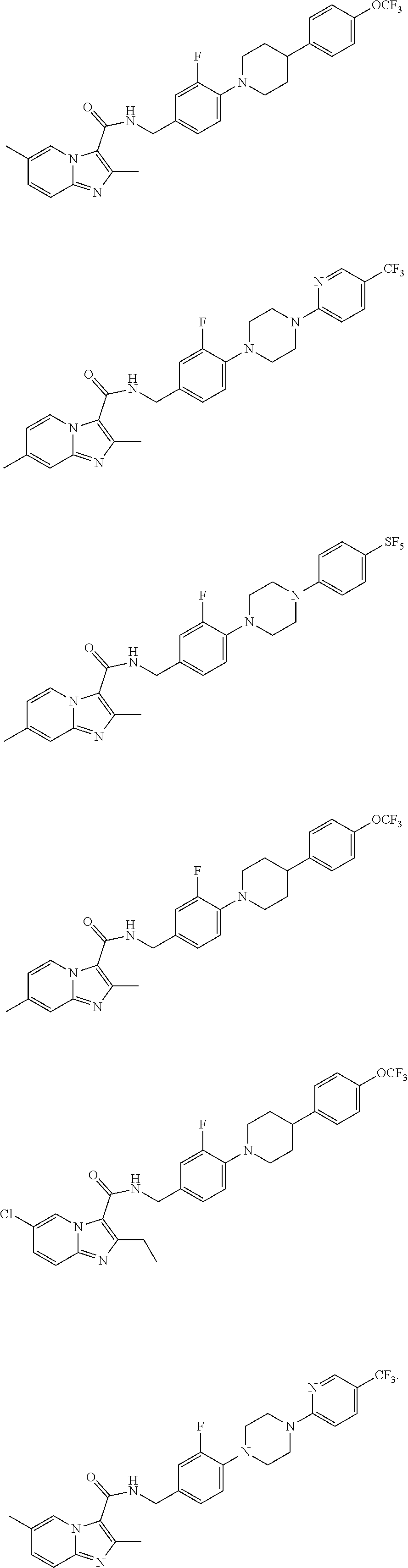

(2) The compound according to the above (1) or its pharmaceutically acceptable salt,
wherein
a group represented by formula:

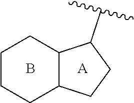

is a group represented by formula:

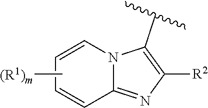

wherein each symbol is as defined in the above (1).
(3) The compound according to the above (1) or its pharmaceutically acceptable salt,
wherein
a group represented by formula:

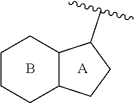

is a group represented by formula:

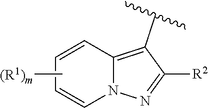

wherein each symbol is as defined in the above (1).
(4) The compound according to the above (2) or its pharmaceutically acceptable salt, wherein R¹ is halogen or substituted or unsubstituted alkyl.
(5) The compound according to the above (3) or its pharmaceutically acceptable salt, wherein R¹ is substituted or unsubstituted alkyloxy or substituted or unsubstituted alkyl.
(6) The compound according to any one of the above (1) to (5) or its pharmaceutically acceptable salt, wherein m is 1.
(7) The compound according to any one of the above (1) to (6) or its pharmaceutically acceptable salt, wherein R² is substituted or unsubstituted alkyl.
(8) The compound according to any one of the above (1) to (7) or its pharmaceutically acceptable salt, wherein R^{3b} is halogen.
(9) The compound according to any one of the above (1) to (8) or its pharmaceutically acceptable salt, wherein R^{3b} and R^{3c} are each independently halogen.
(10) The compound according to any one of the above (1) to (7) or its pharmaceutically acceptable salt, wherein R^{3a} is halogen.
(11) The compound according to any one of the above (1) to (10) or its pharmaceutically acceptable salt, wherein ring C is represented as follows:

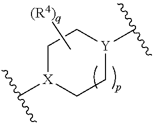

wherein each symbol is as defined in the above (1).
(12) The compound according to any one of the above (1) to (11) or its pharmaceutically acceptable salt, wherein p is 1.
(13) The compound according to any one of the above (1) to (12) or its pharmaceutically acceptable salt, wherein q is 0.
(14) The compound according to any one of the above (1) to (12) or its pharmaceutically acceptable salt, wherein q is 1.
(15) The compound according to any one of the above (1) to (12) or its pharmaceutically acceptable salt, wherein q is 2.
(16) The compound according to the above (14) or its pharmaceutically acceptable salt, wherein ring C is represented as follows:

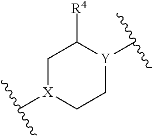

wherein each symbol is as defined in the above (1).
(17) The compound according to the above (15) or its pharmaceutically acceptable salt, wherein ring C is represented as follows:

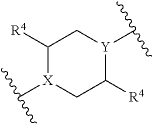

wherein each symbol is as defined in the above (1).
(18) The compound according to any one of the above (1) to (17) or its pharmaceutically acceptable salt, wherein X and Y are N.
(19) The compound according to any one of the above (1) to (17) or its pharmaceutically acceptable salt, wherein one of X and Y is N, and the other of X and Y is CH.
(20) The compound according to any one of the above (1) to (12) or (14) to (19), or its pharmaceutically acceptable salt, wherein R⁴ is each independently substituted or unsubstituted alkyl.
(21) The compound according to any one of the above (1) to (20) or its pharmaceutically acceptable salt, wherein R⁷ is CR^{7C}, and R^{7C} is substituted or unsubstituted alkyl or substituted or unsubstituted alkyloxy.
(22) The compound according to the above (21) or its pharmaceutically acceptable salt, wherein R⁵, R⁶, R⁸ and R⁹ are CH.
(23) The compound according to the above (21) or its pharmaceutically acceptable salt, wherein R⁵ is CR^{5C}, R^{5C} is halogen and R⁶, R⁸ and R⁹ are CH.
(24) The compound according to the above (1) or its pharmaceutically acceptable salt, wherein the compound is selected from compounds (I-1-3), (I-1-25), (I-1-29), (I-1-38), (I-1-39), (I-1-42), (I-1-43), (I-1-45), (I-1-95) and (I-1-118).
(25) The compound according to the above (1) or its pharmaceutically acceptable salt, wherein the compound is selected from compounds (I-1-144), (I-1-149) and (I-2-6).
(26) A pharmaceutical composition comprising the compound according to any one of the above (1) to (25) or its pharmaceutically acceptable salt.
(27) The pharmaceutical composition according to the above (26), for the treatment and/or prevention of mycobacterial infection.
(28) A method for preventing or treating mycobacterial infection, comprising administering the compound to a subject according to any one of the above (1) to (25), or its pharmaceutically acceptable salt.
(29) The compound according to any one of the above (1) to (25), or its pharmaceutically acceptable salt, for the treatment and/or prevention of mycobacterial infection.
(1A) A compound represented by formula (I):

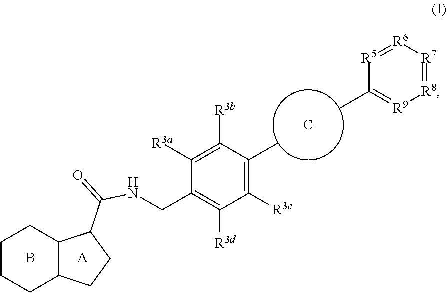

or its pharmaceutically acceptable salt,
wherein
ring A and ring B are represented as follows:

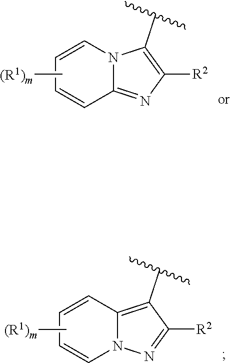

R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted alkylsulfanyl, substituted or unsubstituted alkenylsulfanyl, substituted or unsubstituted alkynylsulfanyl, substituted or unsubstituted alkylsulfinyl, substituted or unsubstituted alkenylsulfinyl, substituted or unsubstituted alkynylsulfinyl, substituted or unsubstituted alkylsulfonyl, substituted or unsubstituted alkenylsulfonyl or substituted or unsubstituted alkynylsulfonyl;
m is 0, 1, 2, 3 or 4;
R² is a hydrogen atom, halogen, cyano, substituted or unsubstituted alkyl, substituted or unsubstituted alkenyl or substituted or unsubstituted alkynyl;
R^{3a}, R^{3b}, R^{3c} and R^{3d} are each independently hydrogen atom, halogen, hydroxy, cyano, substituted or unsubstituted alkyl, substituted or unsubstituted alkenyl or substituted or unsubstituted alkynyl, with the proviso that R^{3a}, R^{3b}, R^{3c} and R^{3d} are not simultaneously hydrogen atom;
ring C is represented as follows:

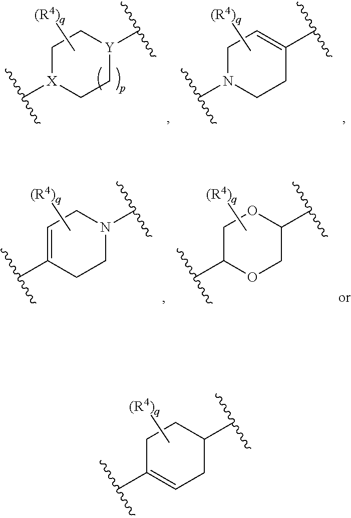

### X is CH or N;

### Y is CH or N;

R⁴ is each independently halogen, hydroxy, cyano, substituted or unsubstituted alkyl, substituted or unsubstituted alkenyl or substituted or unsubstituted alkynyl; two R⁴s attached to adjacent carbon atoms may be taken together with the carbon atoms to which they are attached to form a substituted or unsubstituted 5- to 6-membered non-aromatic carbocycle or a substituted or unsubstituted 5- to 6-membered non-aromatic heterocycle;
two R⁴s attached to a same carbon atom may be taken together with the carbon atom to which they are attached to form a substituted or unsubstituted 3- to 6-membered non-aromatic carbocycle or a substituted or unsubstituted 3- to 6-membered non-aromatic heterocycle;
two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano or substituted or unsubstituted alkyl;
R^{4N} is each independently a hydrogen atom or substituted or unsubstituted alkyl;
p is 0 or 1;
q is 0, 1, 2, 3 or 4;
R⁵ is CR^{5C} or N;
R⁶ is CR^{6C} or N;
R⁷ is CR^{7C} or N;
R⁸ is CR^{8C} or N;
R⁹ is CR^{9C} or N;
with the proviso that R⁵, R⁶, R⁷, R⁸ and R⁹ are not simultaneously N;
R^{5C}, R^{6C}, R^{7C}, R^{8C} and R^{9C} are each independently a hydrogen atom, halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted aromatic carbocyclyloxy, substituted or unsubstituted non-aromatic carbocyclyloxy, substituted or unsubstituted aromatic heterocyclyloxy, substituted or unsubstituted non-aromatic heterocyclyloxy or pentafluorothio;
with the proviso that compounds shown below are excluded:

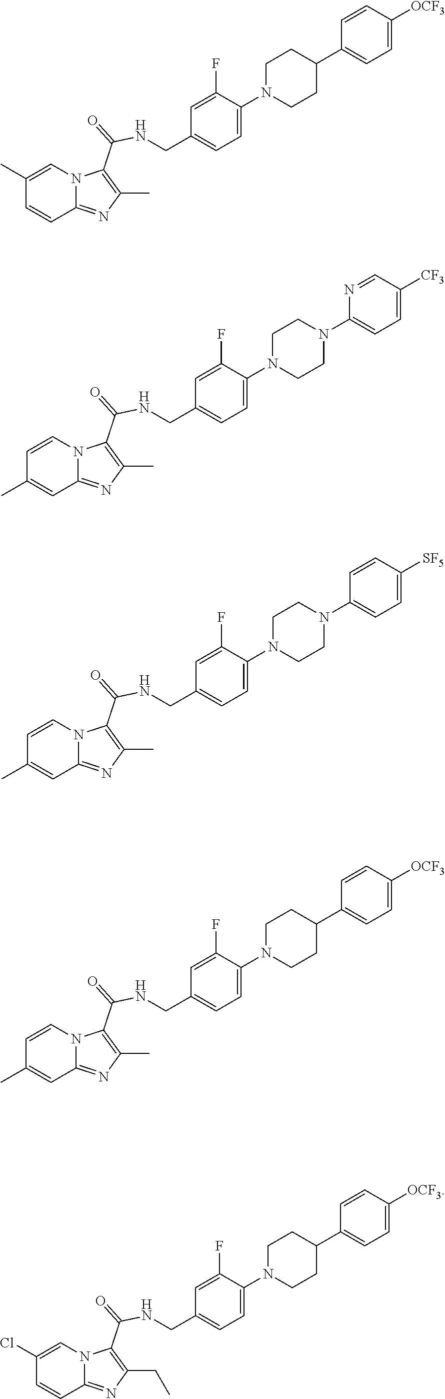

(2A) The compound according to the above (1A) or its pharmaceutically acceptable salt, wherein ring A and ring B are represented as follows:

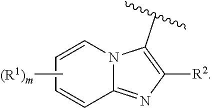

(3A) The compound according to the above (1A) or its pharmaceutically acceptable salt, wherein ring A and ring B are represented as follows:

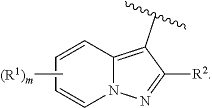

(4A) The compound according to the above (2A) or its pharmaceutically acceptable salt, wherein R¹ is halogen or substituted or unsubstituted alkyl.
(5A) The compound according to the above (3A) or its pharmaceutically acceptable salt, wherein R¹ is substituted or unsubstituted alkyloxy or substituted or unsubstituted alkyl.
(6A) The compound according to any one of the above (1A) to (5A) or its pharmaceutically acceptable salt, wherein m is 1.
(7A) The compound according to any one of the above (1A) to (6A) or its pharmaceutically acceptable salt, wherein R² is substituted or unsubstituted alkyl.
(8A) The compound according to any one of the above (1A) to (7A) or its pharmaceutically acceptable salt, wherein R^{3b} is halogen.
(9A) The compound according to any one of the above (1A) to (8A) or its pharmaceutically acceptable salt, wherein R^{3b} and R^{3c} are each independently halogen.
(10A) The compound according to any one of the above (1A) to (9A) or its pharmaceutically acceptable salt, wherein ring C is represented as follows:

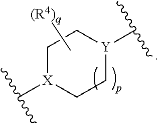

(11A) The compound according to any one of the above (1A) to (10A) or its pharmaceutically acceptable salt, wherein p is 1.
(12A) The compound according to any one of the above (1A) to (11A) or its pharmaceutically acceptable salt, wherein q is 1.
(13A) The compound according to any one of the above (1A) to (12A) or its pharmaceutically acceptable salt, wherein ring C is represented as follows:

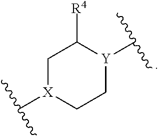

(14A) The compound according to any one of the above (1A) to (13A) or its pharmaceutically acceptable salt, wherein X and Y are N.
(15A) The compound according to any one of the above (1A) to (13A) or its pharmaceutically acceptable salt, wherein one of X and Y is N, and the other of X and Y is CH.
(16A) The compound according to any one of the above (1A) to (15A) or its pharmaceutically acceptable salt, wherein R⁴ is substituted or unsubstituted alkyl.
(17A) The compound according to any one of the above (1A) to (16A) or its pharmaceutically acceptable salt, wherein R⁷ is CR^{7C}, and R^{7C} is substituted or unsubstituted alkyloxy.
(18A) The compound according to the above (17A) or its pharmaceutically acceptable salt, wherein R⁵, R⁶, R⁸ and R⁹ are CH.
(19A) The compound according to the above (17A) or its pharmaceutically acceptable salt, wherein R⁵ is CR^{5C}, R^{5C} is halogen and R⁶, R⁸ and R⁹ are CH.
(20A) The compound according to the above (1A) or its pharmaceutically acceptable salt, wherein the compound is selected from compounds (I-1-3), (I-1-25), (I-1-29), (I-1-38), (I-1-39), (I-1-42), (I-1-43), (I-1-45), (I-1-95) or (I-1-118).
(21A) A pharmaceutical composition comprising the compound according to any one of the above (1A) to (20A) or its pharmaceutically acceptable salt.
(22A) The pharmaceutical composition according to the above (21A), for the treatment and/or prevention of mycobacterial infection.
(23A) A method for preventing or treating mycobacterial infection, comprising administering the compound to a subject according to any one of the above (1A) to (20A), or its pharmaceutically acceptable salt.
(24A) The compound according to any one of the above (1A) to (20A), or its pharmaceutically acceptable salt, for the treatment and/or prevention of mycobacterial infection.

### Effect of the Invention

The compounds of the present invention are useful in the treatment or prevention of a mycobacterial infection, especially non-tuberculous mycobacterial infection.

### DETAILED DESCRIPTION OF SEVERAL EMBODIMENTS OF THE INVENTION

Each term used in this description will be described below. In this description, even when each term is used individually or used together with other terms, the term has the same meaning.

The term, “consisting of” means having only the recited components or elements.

The term, “comprising” means not restricting with components and not excluding undescribed factors.

The use of the word “a” or “an” when used in conjunction with the term “comprising” in the claims and/or the specification may mean “one,” but it is also consistent with the meaning of “one or more,” “at least one,” and “one or more than one.”

The compound represented by formula (I), or its pharmaceutically acceptable salt is described hereinabove and below.

The term “halogen” includes a fluorine atom, a chlorine atom, a bromine atom and an iodine atom. A fluorine atom and a chlorine atom are especially preferable.

The term “alkyl” includes a C1 to C15, preferably C1 to C10, more preferably C1 to C6 and further preferably C1 to C4 linear or branched hydrocarbon group. Examples include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, and n-decyl.

A preferred embodiment of “alkyl” is methyl, ethyl, n-propyl, isopropyl, n-butyl, isobutyl, sec-butyl, tert-butyl or n-pentyl. A more preferred embodiment is methyl, ethyl, n-propyl, isopropyl or tert-butyl.

The term “alkenyl” includes a C2 to C15, preferably a C2 to C10, more preferably a C2 to C6 and further preferably a C2 to C4 linear or branched hydrocarbon group having one or more double bond(s) at any position(s). Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl, and pentadecenyl.

A preferred embodiment of “alkenyl” is vinyl, allyl, propenyl, isopropenyl or butenyl.

The term “alkynyl” includes C2 to C8 straight or branched alkynyl having one or more triple bond(s) in the above “alkyl”, and examples thereof include ethynyl, propynyl, butynyl and the like. Furthermore, an “alkynyl” may have a double bond.

The term “alkyloxy” means a group wherein the above “alkyl” is bonded to an oxygen atom. Examples include methyloxy, ethyloxy, n-propyloxy, isopropyloxy, n-butyloxy, tert-butyloxy, isobutyloxy, sec-butyloxy, pentyloxy, isopentyloxy, and hexyloxy.

A preferred embodiment of “alkyloxy” is methyloxy, ethyloxy, n-propyloxy, isopropyloxy or tert-butyloxy.

The term “alkenyloxy” means a group wherein the above “alkenyl” is bonded to an oxygen atom. Examples include vinyloxy, allyloxy, 1-n-propenyloxy, 2-n-butenyloxy, 2-n-pentenyloxy, 2-n-hexenyloxy, 2-n-heptenyloxy, and 2-n-octenyloxy.

The term “alkynyloxy” means a group wherein the above “alkynyl” is bonded to an oxygen atom. Examples include ethynyloxy, 1-n-propynyloxy, 2-n-propynyloxy, 2-n-butynyloxy, 2-n-pentynyloxy, 2-n-hexynyloxy, 2-n-heptynyloxy, and 2-n-octynyloxy.

The term “aromatic carbocycle” means a cyclic aromatic hydrocarbon ring which is monocyclic or polycyclic having two or more rings. For example, it includes benzene ring, naphthalene ring, anthracene ring, phenanthrene ring and the like.

A preferred embodiment of “aromatic carbocycle” is a benzene ring.

The term “aromatic carbocyclyl” means a cyclic aromatic hydrocarbon group which is monocyclic or polycyclic having two or more rings. For example, it includes phenyl, naphthyl, anthryl, phenanthryl and the like.

A preferred embodiment of “aromatic carbocyclyl” is phenyl.

The term “non-aromatic carbocycle” means a cyclic saturated hydrocarbon ring or a cyclic unsaturated non-aromatic hydrocarbon ring, which is monocyclic or polycyclic having two or more rings. “Non-aromatic carbocycle”, which is polycyclic having two or more rings, includes a fused ring wherein a non-aromatic carbocycle, which is monocyclic or polycyclic having two or more rings, is fused with a ring of the above “aromatic carbocycle”.

In addition, the “non-aromatic carbocycle” also includes a ring having a bridge or a ring to form a spiro ring as follows.

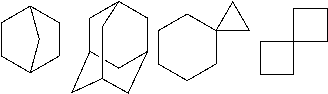

A non-aromatic carbocycle which is monocyclic is preferably C3 to C16, more preferably C3 to C12 and further preferably C3 to C6 carbocycle. For example, it includes cyclopropane, cyclobutane, cyclopentane, cyclohexane, cycloheptane, cyclooctane, cyclononane, cyclodecane, cyclopropene, cyclobutene, cyclopentene, cyclohexene, cycloheptene, cyclohexadiene and the like.

A non-aromatic carbocycle which is polycyclic having two or more rings includes, for example, indane, indene, acenaphthalene, tetrahydronaphthalene, fluorene and the like.

The term “non-aromatic carbocyclyl” means a cyclic saturated hydrocarbon group or a cyclic unsaturated non-aromatic hydrocarbon group, which is monocyclic or polycyclic having two or more rings. “Non-aromatic carbocyclyl”, which is polycyclic having two or more rings, includes a fused ring group wherein a non-aromatic carbocyclyl, which is monocyclic or polycyclic having two or more rings, is fused with a ring of the above “aromatic carbocyclyl”.

In addition, the “non-aromatic carbocyclyl” also includes a group having abridge or a group forming a spiro ring as follows:

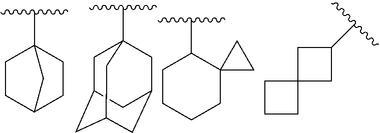

A non-aromatic carbocyclyl which is monocyclic is preferably C3 to C16, more preferably C3 to C12 and further preferably C3 to C6 carbocyclyl. For example, it includes cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptenyl, cyclohexadienyl and the like.

A non-aromatic carbocyclyl which is polycyclic having two or more rings includes, for example, indanyl, indenyl, acenaphthyl, tetrahydronaphthyl, fluorenyl and the like.

The term “aromatic heterocycle” means an aromatic ring, which is monocyclic or polycyclic having two or more rings, containing one or more, same or different, heteroatom(s) selected independently from O, S and N.

“Aromatic heterocycle”, which is polycyclic having two or more rings, includes a fused ring wherein an aromatic heterocycle, which is monocyclic or polycyclic having two or more rings, is fused with a ring of the above “aromatic carbocycle”.

An aromatic heterocycle which is monocyclic is preferably a 5- to 8-membered and more preferably 5- to 6-membered ring. For example, it includes “5-membered aromatic heterocycle” such as pyrrole, imidazole, pyrazole, triazole, tetrazole, furan, thiophene, isoxazole, oxazole, oxadiazole, isothiazole, thiazole, thiadiazole and the like, and “6-membered aromatic heterocycle” such as pyridine, pyridazine, pyrimidine, pyrazine, triazine, and the like.

An aromatic heterocycle which is bicyclic includes, for example, indole, isoindole, indazole, indolizine, quinoline, isoquinoline, cinnoline, phthalazine, quinazoline, naphthyridine, quinoxaline, purine, pteridine, benzimidazole, benzisoxazole, benzoxazole, benzoxadiazole, benzisothiazole, benzothiazole, benzothiadiazole, benzofuran, isobenzofuran, benzothiophene, benzotriazole, pyrazolopyridin, imidazopyridine, triazolopyridine, imidazothiazole, pyrazinopyridazine, oxazolopyridine, thiazolopyridine and the like.

An aromatic heterocycle which is polycyclic having three or more rings includes, for example, carbazole, acridine, xanthene, phenothiazine, phenoxathiine, phenoxazine, dibenzofuran and the like.

The term “aromatic heterocyclyl” means an aromatic cyclyl, which is monocyclic or polycyclic having two or more rings, containing one or more, same or different heteroatom(s) selected independently from O, S and N.

“Aromatic heterocyclyl”, which is polycyclic having two or more rings, includes a fused ring group wherein an aromatic heterocyclyl, which is monocyclic or polycyclic having two or more rings, is fused with a ring of the above “aromatic carbocyclyl”.

An aromatic heterocyclyl which is monocyclic is preferably a 5- to 8-membered and more preferably 5- to 6-membered ring. For example, it includes pyrrolyl, imidazolyl, pyrazolyl, pyridyl, pyridazinyl, pyrimidinyl, pyrazinyl, triazolyl, triazinyl, tetrazolyl, furyl, thienyl, isoxazolyl, oxazolyl, oxadiazolyl, isothiazolyl, thiazolyl, thiadiazolyl and the like.

An aromatic heterocyclyl which is bicyclic includes, for example, indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, pyrazolopyridyl, imidazopyridyl, triazolopyridyl, imidazothiazolyl, pyrazinopyridazinyl, oxazolopyridyl, thiazolopyridyl and the like.

An aromatic heterocyclyl which is polycyclic having three or more rings includes, for example, carbazolyl, acridinyl, xanthenyl, phenothiazinyl, phenoxathiinyl, phenoxazinyl, dibenzofuryl and the like.

The term “non-aromatic heterocycle” means a non-aromatic ring, which is monocyclic or polycyclic having two or more rings, containing one or more, same or different, heteroatom(s) selected independently from O, S and N.

“Non-aromatic heterocycle”, which is polycyclic having two or more rings, includes a fused ring wherein a non-aromatic heterocycle, which is monocyclic or polycyclic having two or more ring(s), is fused with a ring of the above “aromatic carbocycle”, “non-aromatic carbocycle” and/or “aromatic heterocycle”. The non-aromatic heterocycle, which is polycyclic having two or more rings, further includes a fused ring wherein an aromatic heterocycle, which is monocyclic or polycyclic having two or more rings, is fused with a ring of the above “non-aromatic carbocycle”.

In addition, the “non-aromatic heterocycle” also includes a ring having a bridge or a ring forming a spiro ring as follows.

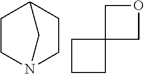

A non-aromatic heterocycle which is monocyclic is preferably a 3- to 8-membered, more preferably 3 to a 6-membered, and more preferably 5- to 6-membered ring. For example, it includes

“5-membered non-aromatic heterocycle” such as thiazolidine, pyrrolidine, pyrroline, imidazolidine, imidazoline, pyrazolidine, pyrazoline, tetrahydrofuran, dihydrothiazole, tetrahydrothiazole, tetrahydroisothiazole, dioxolane, dioxoline and the like,
“6-membered non-aromatic heterocycle” such as dioxane, thiane, piperidine, piperazine, morpholine, thiomorpholine, dihydropyridine, tetrahydropyridine, tetrahydropyran, dihydrooxazine, tetrahydropyridazine, hexahydropyrimidine, thiazine and the like, and,
thiirane, oxirane, oxetane, oxathiolane, azetidine, hexahydroazepine, tetrahydrodiazepine, dioxazine, aziridine, oxepane, thiolane, thiine and the like.

A non-aromatic heterocycle which is polycyclic having two or more rings includes, for example, indoline, isoindoline, chromane, isochromane, dihydrobenzofuran, dihydroisobenzofuran, dihydroquinoline, dihydroisoquinoline, tetrahydroquinoline, tetrahydroisoquinoline and the like.

The term “non-aromatic heterocyclyl” means a non-aromatic cyclyl, which is monocyclic or polycyclic having two or more rings, containing one or more, same or different, heteroatom(s) selected independently from O, S and N.

“Non-aromatic heterocyclyl”, which is polycyclic having two or more rings, includes a fused ring group wherein a non-aromatic heterocycle, which is monocyclic or polycyclic having two or more ring(s), is fused with a ring of the above “aromatic carbocyclyl”, “non-aromatic carbocyclyl” and/or “aromatic heterocyclyl”.

In addition, the “non-aromatic heterocyclyl” also includes a group having a bridge or a group forming a spiro ring as follows:

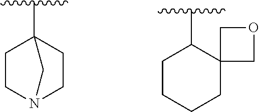 [SMILES: *C12CCN(CC1)C2 ; *C1CCCCC12COC2]

A non-aromatic heterocyclyl which is monocyclic is preferably a 3- to 8-membered and more preferably 5- to 6-membered ring. For example, it includes dioxanyl, thiiranyl, oxiranyl, oxetanyl, oxathiolanyl, azetidinyl, thianyl, thiazolidinyl, pyrrolidinyl, pyrrolinyl, imidazolidinyl, imidazolinyl, pyrazolidinyl, pyrazolinyl, piperidinyl, piperazinyl, morpholinyl, morpholino, thiomorpholinyl, thiomorpholino, dihydropyridyl, tetrahydropyridyl, tetrahydrofuryl, tetrahydropyranyl, dihydrothiazolyl, tetrahydrothiazolyl, tetrahydroisothiazolyl, dihydrooxazinyl, hexahydroazepinyl, tetrahydrodiazepinyl, tetrahydropyridazinyl, hexahydropyrimidinyl, dioxolanyl, dioxazinyl, aziridinyl, dioxolinyl, oxepanyl, thiolanyl, thiinyl, thiazinyl and the like.

A non-aromatic heterocyclyl which is polycyclic having two or more rings includes, for example, indolinyl, isoindolinyl, chromanyl, isochromanyl dihydrobenzofuryl, dihydroisobenzofuryl, dihydroquinolyl, dihydroisoquinolyl, tetrahydroquinolyl, tetrahydroisoquinolyl and the like.

The term “aromatic carbocyclyloxy” means a group wherein the “aromatic carbocycle” is bonded to an oxygen atom. Examples include phenyloxy and naphthyloxy.

The term “non-aromatic carbocyclyloxy” means a group wherein the “non-aromatic carbocycle” is bonded to an oxygen atom. Examples include cyclopropyloxy, cyclohexyloxy, and cyclohexenyloxy.

The term “aromatic heterocyclyloxy” means a group wherein the “aromatic heterocycle” is bonded to an oxygen atom. Examples include pyridyloxy and oxazolyloxy.

The term “non-aromatic heterocyclyloxy” means a group wherein the “non-aromatic heterocycle” is bonded to an oxygen atom. Examples include piperidinyloxy and tetrahydrofuryloxy.

The substituents of “substituted alkyl”, “substituted alkenyl”, “substituted alkynyl”, “substituted alkyloxy”, “substituted alkenyloxy” and “substituted alkynyloxy” include the following substituents. A carbon atom at any positions may be bonded to one or more group(s) selected from the following substituents.

A substituent: halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azido, hydrazino, ureido, amidino, guanidino, trialkylsilyl, alkyloxy, alkenyloxy, alkynyloxy, haloalkyloxy, alkylcarbonyl, alkenylcarbonyl, alkynylcarbonyl, alkylamino, alkenylamino, alkynylamino, alkylsulfonyl, alkenylsulfonyl, alkynylsulfonyl, alkylcarbonylamino, alkenylcarbonylamino, alkynylcarbonylamino, alkylsulfonylamino, alkenylsulfonylamino, alkynylsulfonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkenylsulfinyl, alkynylsulfinyl, alkylcarbamoyl, alkenylcarbamoyl, alkynylcarbamoyl, alkylsulfamoyl, alkenylsulfamoyl, alkynylsulfamoyl, aromatic carbocyclyl, non-aromatic carbocyclyl, aromatic heterocyclyl, non-aromatic heterocyclyl, aromatic carbocyclyloxy, non-aromatic carbocyclyloxy, aromatic heterocyclyloxy, non-aromatic heterocyclyloxy, aromatic carbocyclylcarbonyl, non-aromatic carbocyclylcarbonyl, aromatic heterocyclylcarbonyl, non-aromatic heterocyclylcarbonyl, aromatic carbocyclyloxycarbonyl, non-aromatic carbocyclyloxycarbonyl, aromatic heterocyclyloxycarbonyl, non-aromatic heterocyclyloxycarbonyl, aromatic carbocyclylalkyloxy, non-aromatic carbocyclylalkyloxy, aromatic heterocyclylalkyloxy, non-aromatic heterocyclylalkyloxy, aromatic carbocyclylalkyloxycarbonyl, non-aromatic carbocyclylalkyloxycarbonyl, aromatic heterocyclylalkyloxycarbonyl, non-aromatic heterocyclylalkyloxycarbonyl, aromatic carbocyclylalkylamino, non-aromatic carbocyclylalkylamino, aromatic heterocyclylalkylamino, non-aromatic heterocyclylalkylamino, aromatic carbocyclylsulfanyl, non-aromatic carbocyclylsulfanyl, aromatic heterocyclylsulfanyl, non-aromatic heterocyclylsulfanyl, non-aromatic carbocyclylsulfonyl, aromatic carbocyclylsulfonyl, aromatic heterocyclylsulfonyl, and non-aromatic heterocyclylsulfonyl.

A preferable substituent: halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azido, hydrazino, ureido, amidino, guanidino, trialkylsilyl, alkyloxy, alkenyloxy, alkynyloxy, haloalkyloxy, alkylcarbonyl, alkenylcarbonyl, alkynylcarbonyl, alkylamino, alkenylamino, alkynylamino, alkylsulfonyl, alkenylsulfonyl, alkynylsulfonyl, alkylcarbonylamino, alkenylcarbonylamino, alkynylcarbonylamino, alkylsulfonylamino, alkenylsulfonylamino, alkynylsulfonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkenylsulfinyl, alkynylsulfinyl, alkylcarbamoyl, alkenylcarbamoyl, alkynylcarbamoyl, alkylsulfamoyl, alkenylsulfamoyl, and alkynylsulfamoyl.

A more preferable substituent: halogen, hydroxy, amino, cyano, alkyloxy, alkenyloxy, alkynyloxy, haloalkyloxy, alkylamino, alkenylamino, and alkynylamino.

An especially preferable substituent: halogen and non-aromatic carbocyclyl.

The substituents on the ring of “aromatic carbocycle”, “non-aromatic carbocycle”, “aromatic heterocycle”, and “non-aromatic heterocycle” part of “a substituted or unsubstituted 5- to 6-membered non-aromatic carbocycle formed by two R⁴s attached to adjacent carbon atoms, together with the carbon atoms to which they are attached”, “a substituted or unsubstituted 5- to 6-membered non-aromatic heterocycle formed by two R⁴s attached to adjacent carbon atoms, together with the carbon atoms to which they are attached”, “a substituted or unsubstituted 3- to 6-membered non-aromatic carbocycle formed by two R⁴s attached to a same carbon atom, together with the carbon atom to which they are attached”, “a substituted or unsubstituted 3- to 6-membered non-aromatic heterocycle formed by two R⁴s attached to a same carbon atom, together with the carbon atom to which they are attached”, “substituted or unsubstituted aromatic carbocyclyloxy”, “substituted or unsubstituted non-aromatic carbocyclyloxy”, “substituted or unsubstituted aromatic heterocyclyloxy” and “substituted or unsubstituted non-aromatic heterocyclyloxy”, include the following substituents. An atom at any position(s) on the ring may be bonded to one or more group(s) selected from the following substituents.

A substituent: halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azido, hydrazino, ureido, amidino, guanidino, trialkylsilyl, alkyl, alkenyl, alkynyl, haloalkyl, alkyloxy, alkenyloxy, alkynyloxy, haloalkyloxy, alkyloxyalkyl, alkyloxyalkyloxy, alkylcarbonyl, alkenylcarbonyl, alkynylcarbonyl, alkylamino, alkenylamino, alkynylamino, alkylsulfonyl, alkenylsulfonyl, alkynylsulfonyl, alkylcarbonylamino, alkenylcarbonylamino, alkynylcarbonylamino, alkylsulfonylamino, alkenylsulfonylamino, alkynylsulfonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkenylsulfinyl, alkynylsulfinyl, alkylcarbamoyl, alkenylcarbamoyl, alkynylcarbamoyl, alkylsulfamoyl, alkenylsulfamoyl, alkynylsulfamoyl, aromatic carbocyclyl, non-aromatic carbocyclyl, aromatic heterocyclyl, non-aromatic heterocyclyl, aromatic carbocyclyloxy, non-aromatic carbocyclyloxy, aromatic heterocyclyloxy, non-aromatic heterocyclyloxy, aromatic carbocyclylcarbonyl, non-aromatic carbocyclylcarbonyl, aromatic heterocyclylcarbonyl, non-aromatic heterocyclylcarbonyl, aromatic carbocyclyloxycarbonyl, non-aromatic carbocyclyloxycarbonyl, aromatic heterocyclyloxycarbonyl, non-aromatic heterocyclyloxycarbonyl, aromatic carbocyclylalkyl, non-aromatic carbocyclylalkyl, aromatic heterocyclylalkyl, non-aromatic heterocyclylalkyl, aromatic carbocyclylalkyloxy, non-aromatic carbocyclylalkyloxy, aromatic heterocyclylalkyloxy, non-aromatic heterocyclylalkyloxy, aromatic carbocyclylalkyloxycarbonyl, non-aromatic carbocyclylalkyloxycarbonyl, aromatic heterocyclylalkyloxycarbonyl, non-aromatic heterocyclylalkyloxycarbonyl, aromatic carbocyclylalkyloxyalkyl, non-aromatic carbocyclylalkyloxyalkyl, aromatic heterocyclylalkyloxyalkyl, non-aromatic heterocyclylalkyloxyalkyl, aromatic carbocyclylalkylamino, non-aromatic carbocyclylalkylamino, aromatic heterocyclylalkylamino, non-aromatic heterocyclylalkylamino, aromatic carbocyclylsulfanyl, non-aromatic carbocyclylsulfanyl, aromatic heterocyclylsulfanyl, non-aromatic heterocyclylsulfanyl, non-aromatic carbocyclylsulfonyl, aromatic carbocyclylsulfonyl, aromatic heterocyclylsulfonyl, and non-aromatic heterocyclylsulfonyl.

A preferable substituent: halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azido, hydrazino, ureido, amidino, guanidino, trialkylsilyl, alkyloxy, alkenyloxy, alkynyloxy, haloalkyloxy, alkylcarbonyl, alkenylcarbonyl, alkynylcarbonyl, alkylamino, alkenylamino, alkynylamino, alkylsulfonyl, alkenylsulfonyl, alkynylsulfonyl, alkylcarbonylamino, alkenylcarbonylamino, alkynylcarbonylamino, alkylsulfonylamino, alkenylsulfonylamino, alkynylsulfonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkenylsulfinyl, alkynylsulfinyl, alkylcarbamoyl, alkenylcarbamoyl, alkynylcarbamoyl, alkylsulfamoyl, alkenylsulfamoyl, and alkynylsulfamoyl.

A more preferable substituent: halogen, hydroxy, alkyl, alkenyl, alkynyl, alkyloxy, alkenyloxy, alkynyloxy, haloalkyl, and haloalkyloxy.

An especially preferable substituent: halogen, alkyl, haloalkyl, and haloalkyloxy.

The term “haloalkyl” includes a group wherein one or more hydrogen atom(s) attached to a carbon atom of the above “alkyl” is replaced with the above “halogen”. Examples include monofluoromethyl, monofluoroethyl, monofluoro-n-propyl, 2,2,3,3,3-n-pentafluoropropyl, monochloromethyl, trifluoromethyl, trichloromethyl, 2,2,2-trifluoroethyl, 2,2,2-trichloroethyl, 1,2-dibromoethyl, and 1,1,1-trifluoro-n-propan-2-yl.

A preferred embodiment of “haloalkyl” is trifluoromethyl and trichloromethyl.

The term “haloalkyloxy” means a group wherein the above “haloalkyl” is bonded to an oxygen atom. Examples include monofluoromethoxy, monofluoroethoxy, trifluoromethoxy, trichloromethoxy, trifluoroethoxy, and trichloroethoxy.

A preferred embodiment of “haloalkyloxy” is trifluoromethoxy and trichloromethoxy.

The term “alkylcarbonyl” means a group wherein the above “alkyl” is bonded to a carbonyl group. Examples include methylcarbonyl, ethylcarbonyl, n-propylcarbonyl, isopropylcarbonyl, n-butylcarbonyl, tert-butylcarbonyl, isobutylcarbonyl, sec-butylcarbonyl, n-pentylcarbonyl, isopentylcarbonyl, and n-hexylcarbonyl.

A preferred embodiment of “alkylcarbonyl” is methylcarbonyl, ethylcarbonyl and n-propylcarbonyl.

The term “alkenylcarbonyl” means a group wherein the above “alkenyl” is bonded to a carbonyl group. Examples include vinylcarbonyl, allylcarbonyl and n-propenylcarbonyl.

The term “alkynylcarbonyl” means a group wherein the above “alkynyl” is bonded to a carbonyl group. Examples include ethynylcarbonyl and n-propynylcarbonyl.

The term “alkylamino” means a group wherein one or two hydrogen atom(s) attached to a nitrogen atom of an amino group is(are) replaced with the above “alkyl”. Examples include methylamino, dimethylamino, ethylamino, diethylamino, isopropylamino, N,N-diisopropylamino, and N-methyl-N-ethylamino.

A preferred embodiment of “alkylamino” is methylamino and ethylamino.

The term “alkylsulfonyl” means a group wherein the above “alkyl” is bonded to a sulfonyl group. Examples include methylsulfonyl, ethylsulfonyl, n-propylsulfonyl, isopropylsulfonyl, n-butylsulfonyl, tert-butylsulfonyl, isobutylsulfonyl, and sec-butylsulfonyl.

A preferred embodiment of “alkylsulfonyl” is methylsulfonyl and ethylsulfonyl.

The term “alkenylsulfonyl” means a group wherein the above “alkenyl” is bonded to a sulfonyl group. Examples include vinylsulfonyl, allylsulfonyl, and n-propenylsulfonyl.

The term “alkynylsulfonyl” means a group wherein the above “alkynyl” is bonded to a sulfonyl group. Examples include ethynylsulfonyl, and n-propynylsulfonyl.

The term “alkylcarbonylamino” means a group wherein one or two hydrogen atom(s) attached to a nitrogen atom of an amino group is(are) replaced with the above “alkylcarbonyl”. Examples include methylcarbonylamino, dimethylcarbonylamino, ethylcarbonylamino, diethylcarbonylamino, n-propylcarbonylamino, isopropylcarbonylamino, N,N-diisopropylcarbonylamino, n-butylcarbonylamino, tert-butylcarbonylamino, isobutylcarbonylamino, and sec-butylcarbonylamino.

The term “alkylsulfonylamino” means a group wherein one or two hydrogen atom(s) attached to a nitrogen atom of an amino group is(are) replaced with the above “alkylsulfonyl”. Examples include methylsulfonylamino, dimethylsulfonylamino, ethylsulfonylamino, diethylsulfonylamino, n-propylsulfonylamino, isopropylsulfonylamino, N,N-diisopropylsulfonylamino, n-butylsulfonylamino, tert-butylsulfonylamino, isobutylsulfonylamino, and sec-butylsulfonylamino.

A preferred embodiment of “alkylsulfonylamino” is methylsulfonylamino and ethylsulfonylamino.

The term “alkylimino” means a group wherein a hydrogen atom attached to a nitrogen atom of an imino group is replaced with the above “alkyl”. Examples include methylimino, ethylimino, n-propylimino, and isopropylimino.

The term “alkenylimino” means a group wherein a hydrogen atom attached to a nitrogen atom of an imino group is replaced with the above “alkenyl”. Examples include ethylenylimino, and n-propenylimino.

The term “alkynylimino” means a group wherein a hydrogen atom attached to a nitrogen atom of an imino group is replaced with the above “alkynyl”. Examples include ethynylimino, and n-propynylimino.

The term “alkylcarbonylimino” means a group wherein a hydrogen atom attached to a nitrogen atom of an imino group is replaced with the above “alkylcarbonyl”. Examples include methylcarbonylimino, ethylcarbonylimino, n-propylcarbonylimino, and isopropylcarbonylimino.

The term “alkenylcarbonylimino” means a group wherein a hydrogen atom attached to a nitrogen atom of an imino group is replaced with the above “alkenylcarbonyl”. Examples include ethylenylcarbonylimino, and n-propenylcarbonylimino.

The term “alkynylcarbonylimino” means a group wherein a hydrogen atom attached to a nitrogen atom of an imino group is replaced with the above “alkynylcarbonyl”. Examples include ethynylcarbonylimino and n-propynylcarbonylimino.

The term “alkyloxyimino” means a group wherein a hydrogen atom attached to a nitrogen atom of an imino group is replaced with the above “alkyloxy”. Examples include methyloxyimino, ethyloxyimino, n-propyloxyimino, and isopropyloxyimino.

The term “alkenyloxyimino” means a group wherein a hydrogen atom attached to a nitrogen atom of an imino group is replaced with the above “alkenyloxy”. Examples include ethylenyloxyimino, and n-propenyloxyimino.

The term “alkynyloxyimino” means a group wherein a hydrogen atom attached to a nitrogen atom of an imino group is replaced with the above “alkynyloxy”. Examples include ethynyloxyimino, and n-propynyloxyimino.

The term “alkylcarbonyloxy” means a group wherein the above “alkylcarbonyl” is bonded to an oxygen atom. Examples include methylcarbonyloxy, ethylcarbonyloxy, n-propylcarbonyloxy, isopropylcarbonyloxy, tert-butylcarbonyloxy, isobutylcarbonyloxy, and sec-butylcarbonyloxy.

A preferred embodiment of “alkylcarbonyloxy” is methylcarbonyloxy and ethylcarbonyloxy.

The term “alkenylcarbonyloxy” means a group wherein the above “alkenylcarbonyl” is bonded to an oxygen atom. Examples include ethylenylcarbonyloxy and n-propenylcarbonyloxy.

The term “alkynylcarbonyloxy” means a group wherein the above “alkynylcarbonyl” is bonded to an oxygen atom. Examples include ethynylcarbonyloxy and n-propynylcarbonyloxy.

The term “alkyloxycarbonyl” means a group wherein the above “alkyloxy” is bonded to a carbonyl group. Examples include methyloxycarbonyl, ethyloxycarbonyl, n-propyloxycarbonyl, isopropyloxycarbonyl, n-butyloxycarbonyl, tert-butyloxycarbonyl, isobutyloxycarbonyl, sec-butyloxycarbonyl, n-pentyloxycarbonyl, isopentyloxycarbonyl, and n-hexyloxycarbonyl.

A preferred embodiment of “alkyloxycarbonyl” is methyloxycarbonyl, ethyloxycarbonyl and n-propyloxycarbonyl.

The term “alkenyloxycarbonyl” means a group wherein the above “alkenyloxy” is bonded to a carbonyl group. Examples include ethylenyloxycarbonyl and n-propenyloxycarbonyl.

The term “alkynyloxycarbonyl” means a group wherein the above “alkynyloxy” is bonded to a carbonyl group. Examples include ethynyloxycarbonyl and n-propynyloxycarbonyl.

The term “alkylsulfanyl” means a group wherein a hydrogen atom attached to a sulfur atom of a sulfanyl group is replaced with the above “alkyl”. Examples include methylsulfanyl, ethylsulfanyl, n-propylsulfanyl, and isopropylsulfanyl.

The term “alkenylsulfanyl” means a group wherein a hydrogen atom attached to a sulfur atom of a sulfanyl group is replaced with the above “alkenyl”. Examples include ethylenylsulfanyl, and n-propenylsulfanyl.

The term “alkynylsulfanyl” means a group wherein a hydrogen atom attached to a sulfur atom of a sulfanyl group is replaced with the above “alkynyl”. Examples include ethynylsulfanyl, and n-propynylsulfanyl.

The term “alkylsulfinyl” means a group wherein the above “alkyl” is bonded to a sulfinyl group. Examples include methylsulfinyl, ethylsulfinyl, n-propylsulfinyl, and isopropylsulfinyl.

The term “alkenylsulfinyl” means a group wherein the above “alkenyl” is bonded to a sulfinyl group. Examples include ethylenylsulfinyl, and n-propenylsulfinyl.

The term “alkynylsulfinyl” means a group wherein the above “alkynyl” is bonded to a sulfinyl group. Examples include ethynylsulfinyl and n-propynylsulfinyl.

The term “alkylcarbamoyl” means a group wherein one or two hydrogen atom(s) attached to a nitrogen atom of a carbamoyl group is(are) replaced with the above “alkyl”. Examples include methylcarbamoyl, dimethylcarbamoyl, ethylcarbamoyl, and diethylcarbamoyl.

The term “alkylsulfamoyl” means a group wherein one or two hydrogen atom(s) attached to a nitrogen atom of a sulfamoyl group is(are) replaced with the above “alkyl”. Examples include methylsulfamoyl, dimethylsulfamoyl, ethylsulfamoyl, and diethylsulfamoyl.

The term “aromatic carbocyclylcarbonyl” means a group wherein the “aromatic carbocycle” is bonded to a carbonyl group. Examples include phenylcarbonyl and naphthylcarbonyl.

The term “non-aromatic carbocyclylcarbonyl” means a group wherein the “non-aromatic carbocycle” is bonded to a carbonyl group. Examples include cyclopropylcarbonyl, cyclohexylcarbonyl, and cyclohexenylcarbonyl.

The term “non-aromatic carbocyclylcarbonyloxy” means a group wherein the “non-aromatic carbocyclylcarbonyl” is bonded to an oxygen atom. Examples include cyclopropylcarbonyloxy, cyclohexylcarbonyloxy, and cyclohexenylcarbonyloxy.

The term “aromatic heterocyclylcarbonyl” means a group wherein the “aromatic heterocycle” is bonded to a carbonyl group. Examples include pyridylcarbonyl and oxazolylcarbonyl.

The term “non-aromatic heterocyclylcarbonyl” means a group wherein the “non-aromatic heterocycle” is bonded to a carbonyl group. Examples include piperidinylcarbonyl, and tetrahydrofurylcarbonyl.

The term “aromatic carbocyclyloxycarbonyl” means a group wherein the “aromatic carbocyclyloxy” is bonded to a carbonyl group. Examples include phenyloxycarbonyl and naphthyloxycarbonyl.

The term “non-aromatic carbocyclyloxycarbonyl” means a group wherein the “non-aromatic carbocyclyloxy” is bonded to a carbonyl group. Examples include cyclopropyloxycarbonyl, cyclohexyloxycarbonyl, and cyclohexenyloxycarbonyl.

The term “aromatic heterocyclyloxycarbonyl” means a group wherein the “aromatic heterocyclyloxy” is bonded to a carbonyl group. Examples include pyridyloxycarbonyl and oxazolyloxycarbonyl.

The term “non-aromatic heterocyclyloxycarbonyl” means a group wherein the “non-aromatic heterocyclyloxy” is bonded to a carbonyl group. Examples include piperidinyloxycarbonyl, and tetrahydrofuryloxycarbonyl.

The term “aromatic carbocyclylalkyloxy” means an alkyloxy substituted with one or more “aromatic carbocyclyl” described above. Examples include benzyloxy, phenethyloxy, phenyl-n-propyloxy, benzhydryloxy, trityloxy, naphthylmethyloxy, and a group of the following formula with either the R or S stereochemistry or the racemate:

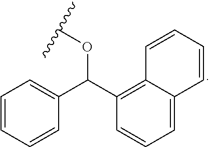 [SMILES: *OC(c1ccccc1)c1cccc2ccccc12]

The term “non-aromatic carbocyclylalkyloxy” means an alkyloxy substituted with one or more “non-aromatic carbocyclyl” described above. The “non-aromatic carbocyclylalkyloxy” also includes “non-aromatic carbocyclylalkyloxy” wherein the alkyl part is substituted with the above “aromatic carbocyclyl”. Examples include cyclopropylmethyloxy, cyclobutylmethyloxy, cyclopenthylmethyloxy, cyclohexylmethyloxy, and a group of the following formula with either the R or S stereochemistry or the racemate:

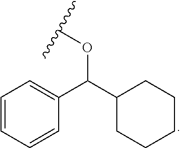 [SMILES: *OC(c1ccccc1)C1CCCCC1]

The term “aromatic heterocyclylalkyloxy” means an alkyloxy substituted with one or more “aromatic heterocyclyl” described above. The “aromatic heterocyclylalkyloxy” also includes “aromatic heterocyclylalkyloxy” wherein the alkyl part is substituted with the above “aromatic carbocyclyl” and/or “non-aromatic carbocyclyl”. Examples include pyridylmethyloxy, furanylmethyloxy, imidazolylmethyloxy, indolylmethyloxy, benzothiophenylmethyloxy, oxazolylmethyloxy, isoxazolylmethyloxy, thiazolylmethyloxy, isothiazolylmethyloxy, pyrazolylmethyloxy, isopyrazolylmethyloxy, pyrrolidinylmethyloxy, benzoxazolylmethyloxy, and groups of the following formulae with either the R or S stereochemistry or the racemate:

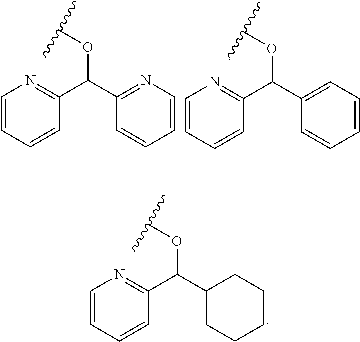

The term “non-aromatic heterocyclylalkyloxy” means an alkyloxy substituted with one or more “non-aromatic heterocyclyl” described above. The “non-aromatic heterocyclylalkyloxy” also includes “non-aromatic heterocyclylalkyloxy” wherein the alkyl part is substituted with the above “aromatic carbocyclyl”, “non-aromatic carbocyclyl” and/or “aromatic heterocyclyl”. Examples include tetrahydropyranylmethyloxy, morpholinylmethyloxy, morpholinylethyloxy, piperidinylmethyloxy, piperazinylmethyloxy, and groups of the following formulae with either the R or S stereochemistry or the racemate:

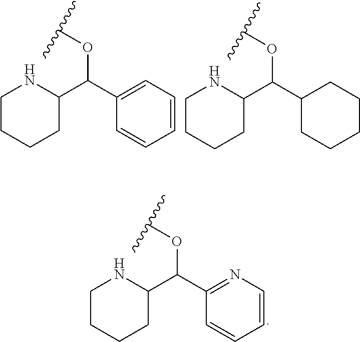

The term “aromatic carbocyclylalkyloxycarbonyl” means an alkyloxycarbonyl substituted with one or more “aromatic carbocyclyl” described above. Examples include benzyloxycarbonyl, phenethyloxycarbonyl, phenyl-n-propyloxycarbonyl, benzhydryloxycarbonyl, trityloxycarbonyl, naphthylmethyloxycarbonyl, and a group of the following formula with either the R or stereochemistry or the racemate

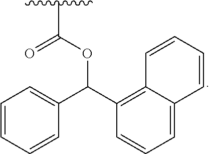 [SMILES: *C(=O)OC(c1ccccc1)c1cccc2ccccc12]

The term “non-aromatic carbocyclylalkyloxycarbonyl” means an alkyloxycarbonyl substituted with one or more “non-aromatic carbocyclyl” described above. The “non-aromatic carbocyclylalkyloxycarbonyl” also includes “non-aromatic carbocyclylalkyloxycarbonyl” wherein the alkyl part is substituted with the above “aromatic carbocyclyl”. Examples include cyclopropylmethyloxycarbonyl, cyclobutylmethyloxycarbonyl, cyclopenthylmethyloxycarbonyl, cyclohexylmethyloxycarbonyl, and a group of the following formula with either the R or S stereochemistry or the racemate:

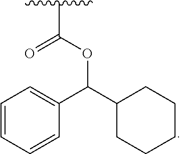 [SMILES: *C(=O)OC(c1ccccc1)C1CCCCC1]

The term “aromatic heterocyclylalkyloxycarbonyl” means an alkyloxycarbonyl substituted with one or more “aromatic heterocyclyl” described above. The “aromatic heterocyclylalkyloxycarbonyl” also include “aromatic heterocyclylalkyloxycarbonyl” wherein the alkyl part is substituted with the above “aromatic carbocyclyl” and/or “non-aromatic carbocyclyl”. Examples include pyridylmethyloxycarbonyl, furanylmethyloxycarbonyl, imidazolylmethyloxycarbonyl, indolylmethyloxycarbonyl, benzothiophenylmethyloxycarbonyl, oxazolylmethyloxycarbonyl, isoxazolylmethyloxycarbonyl, thiazolylmethyloxycarbonyl, isothiazolylmethyloxycarbonyl, pyrazolylmethyloxycarbonyl, isopyrazolylmethyloxycarbonyl, pyrrolidinylmethyloxycarbonyl, benzoxazolylmethyloxycarbonyl, and groups of the following formulae with either the R or S stereochemistry or the racemate:

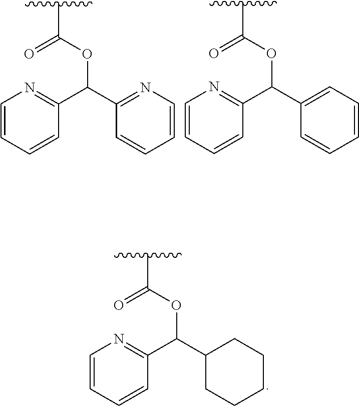

The term “non-aromatic heterocyclylalkyloxycarbonyl” means an alkyloxycarbonyl substituted with one or more “non-aromatic heterocyclyl” described above. The “non-aromatic heterocyclylalkyloxycarbonyl” also includes “non-aromatic heterocyclylalkyloxycarbonyl” wherein the alkyl part is substituted with the above “aromatic carbocyclyl”, “non-aromatic carbocyclyl” and/or “aromatic heterocyclyl”. Examples include tetrahydropyranylmethyloxycarbonyl, morpholinylethyloxycarbonyl, piperidinylmethyloxycarbonyl, piperazinylmethyloxycarbonyl, and groups of the following formulae with either the R or S stereochemistry or the racemate:

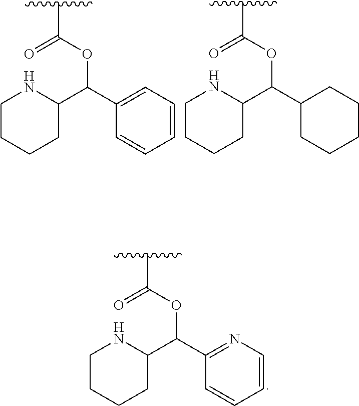

The term “aromatic carbocyclylalkylamino” means a group wherein one or two hydrogen atom(s) attached to a nitrogen atom of an amino group is(are) replaced with the above “aromatic carbocyclylalkyl”. Examples include benzylamino, phenethylamino, phenylpropylamino, benzhydrylamino, tritylamino, naphthylmethylamino, and dibenzylamino.

The term “non-aromatic carbocyclylalkylamino” means a group wherein one or two hydrogen atom(s) attached to a nitrogen atom of an amino group is(are) replaced with the above “non-aromatic carbocyclylalkyl”. Examples include cyclopropylmethylamino, cyclobutylmethylamino, cyclopentylmethylamino, and cyclohexylmethylamino.

The term “aromatic heterocyclylalkylamino” means a group wherein one or two hydrogen atom(s) attached to a nitrogen atom of an amino group is(are) replaced with the above “aromatic heterocyclylalkyl”. Examples include pyridylmethylamino, furanylmethylamino, imidazolylmethylamino, indolylmethylamino, benzothiophenylmethylamino, oxazolylmethylamino, isoxazolylmethylamino, thiazolylmethylamino, isothiazolylmethylamino, pyrazolylmethylamino, isopyrazolylmethylamino, pyrrolylmethylamino, and benzoxazolylmethylamino.

The term “non-aromatic heterocyclylalkylamino” means a group wherein one or two hydrogen atom(s) attached to a nitrogen atom of an amino group is(are) replaced with the above “non-aromatic heterocyclylalkyl”. Examples include tetrahydropyranylmethylamino, morpholinylethylamino, piperidinylmethylamino, and piperazinylmethylamino.

The term “aromatic carbocyclylsulfanyl” means a group wherein a hydrogen atom attached to a sulfur atom of a sulfanyl group is replaced with the “aromatic carbocycle”. Examples include phenylsulfanyl and naphthylsulfanyl.

The term “non-aromatic carbocyclylsulfanyl” means a group wherein a hydrogen atom attached to a sulfur atom of a sulfanyl group is replaced with the “non-aromatic carbocycle”. Examples include cyclopropylsulfanyl, cyclohexylsulfanyl, and cyclohexenylsulfanyl.

The term “aromatic heterocyclylsulfanyl” means a group wherein a hydrogen atom attached to a sulfur atom of a sulfanyl group is replaced with the “aromatic heterocycle”. Examples include pyridylsulfanyl and oxazolylsulfanyl.

The term “non-aromatic heterocyclylsulfanyl” means a group wherein a hydrogen atom attached to a sulfur atom of a sulfanyl group is replaced with the “non-aromatic heterocycle”. Examples include piperidinylsulfanyl and tetrahydrofurylsulfanyl.

The term “non-aromatic carbocyclylsulfonyl” means a group wherein the “non-aromatic carbocycle” is bonded to a sulfonyl group. Examples include cyclopropylsulfonyl, cyclohexylsulfonyl, and cyclohexenylsulfonyl.

The term “aromatic carbocyclylsulfonyl” means a group wherein the “aromatic carbocycle” is bonded to a sulfonyl group. Examples include phenylsulfonyl and naphthylsulfonyl.

The term “aromatic heterocyclylsulfonyl” means a group wherein the “aromatic heterocycle” is bonded to a sulfonyl group. Examples include pyridylsulfonyl and oxazolylsulfonyl.

The term “non-aromatic heterocyclylsulfonyl” means a group wherein the “non-aromatic heterocycle” is bonded to a sulfonyl group. Examples include piperidinylsulfonyl and tetrahydrofurylsulfonyl.

The term “alkyloxyalkyl” means a group wherein the above “alkyloxy” is bonded to the above “alkyl”. Examples include methoxymethyl, methoxyethyl, and ethoxymethyl.

The term “alkyloxyalkyloxy” means a group wherein the above “alkyloxy” is bonded to the above “alkyloxy”. Examples include methoxymethoxy, methoxyethoxy, ethoxymethoxy, and ethoxyethoxy.

The term “aromatic carbocyclylalkyl” means an alkyl substituted with one or more “aromatic carbocyclyl” described above. Examples include benzyl, phenethyl, phenyl-n-propyl, benzhydryl, trityl, naphthylmethyl, and a group of the following formula with either the R or S stereochemistry or the racemate:

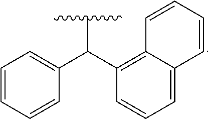 [SMILES: *C(c1ccccc1)c1cccc2ccccc12]

A preferred embodiment of “aromatic carbocyclylalkyl” is benzyl, phenethyl or benzhydryl.

The term “non-aromatic carbocyclylalkyl” means an alkyl substituted with one or more “non-aromatic carbocyclyl” described above. The “non-aromatic carbocyclylalkyl” also includes “non-aromatic carbocyclylalkyl” wherein the alkyl part is substituted with the above “aromatic carbocyclyl”. Examples include cyclopropylmethyl, cyclobutylmethyl, cyclopenthylmethyl, cyclohexylmethyl, and a group of the following formula with either the R or S stereochemistry or the racemate

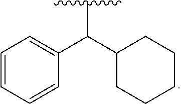 [SMILES: *C(c1ccccc1)C1CCCCC1]

The term “aromatic heterocyclylalkyl” means an alkyl substituted with one or more “aromatic heterocyclyl” described above. The “aromatic heterocyclylalkyl” also includes “aromatic heterocyclylalkyl” wherein the alkyl part is substituted with the above “aromatic carbocyclyl” and/or “non-aromatic carbocyclyl”. Examples include pyridylmethyl, furanylmethyl, imidazolylmethyl, indolylmethyl, benzothiophenylmethyl, oxazolylmethyl, isoxazolylmethyl, thiazolylmethyl, isothiazolylmethyl, pyrazolylmethyl, isopyrazolylmethyl, pyrrolidinylmethyl, benzoxazolylmethyl, and groups of the following formulae with either the R or S stereochemistry or the racemate:

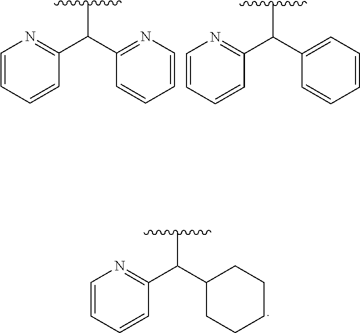

The term “non-aromatic heterocyclylalkyl” means an alkyl substituted with one or more “non-aromatic heterocyclyl” described above. The “non-aromatic heterocyclylalkyl” also includes “non-aromatic heterocyclylalkyl” wherein the alkyl part is substituted with the above “aromatic carbocyclyl”, “non-aromatic carbocyclyl” and/or “aromatic heterocyclyl”. Examples include tetrahydropyranylmethyl, morpholinylethyl, piperidinylmethyl, piperazinylmethyl, and groups of the following formulae with either the R or S stereochemistry or the racemate:

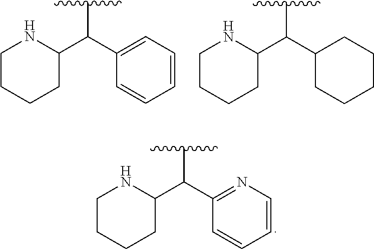

The term “aromatic carbocyclylalkyloxyalkyl” means an alkyloxyalkyl substituted with one or more “aromatic carbocyclyl” described above. Examples include benzyloxymethyl, phenethyloxymethyl, phenylpropyloxymethyl, benzhydryloxymethyl, trityloxymethyl, naphthylmethyloxymethyl, and a group of the following formula with either the R or S stereochemistry or the racemate:

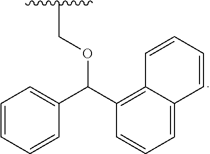 [SMILES: *COC(c1ccccc1)c1cccc2ccccc12]

The term “non-aromatic carbocyclylalkyloxyalkyl” means an alkyloxyalkyl substituted with one or more “non-aromatic carbocyclyl” described above. The “non-aromatic carbocyclylalkyloxyalkyl” also includes “non-aromatic carbocyclylalkyloxyalkyl” wherein the alkyl part bonded to the non-aromatic carbocycle is substituted with the above “aromatic carbocyclyl”. Examples include cyclopropylmethyloxymethyl, cyclobutylmethyloxymethyl, cyclopenthylmethyloxymethyl, cyclohexylmethyloxymethyl, and a group of the following formula with either the R or S stereochemistry or the racemate:

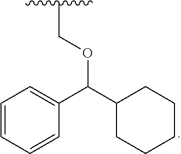 [SMILES: *COC(c1ccccc1)C1CCCCC1]

The term “aromatic heterocyclylalkyloxyalkyl” means an alkyloxyalkyl substituted with one or more “aromatic heterocyclyl” described above. The “aromatic heterocyclylalkyloxyalkyl” also includes “aromatic heterocyclylalkyloxyalkyl” wherein the alkyl part bonded to the aromatic heterocycle is substituted with the above “aromatic carbocyclyl” and/or “non-aromatic carbocyclyl”. Examples include pyridylmethyloxymethyl, furanylmethyloxymethyl, imidazolylmethyloxymethyl, indolylmethyloxymethyl, benzothiophenylmethyloxymethyl, oxazolylmethyloxymethyl, isoxazolylmethyloxymethyl, thiazolylmethyloxymethyl, isothiazolylmethyloxymethyl, pyrazolylmethyloxymethyl, isopyrazolylmethyloxymethyl, pyrrolidinylmethyloxymethyl, benzoxazolylmethyloxymethyl, and groups of the following formulae with either the R or S stereochemistry or the racemate:

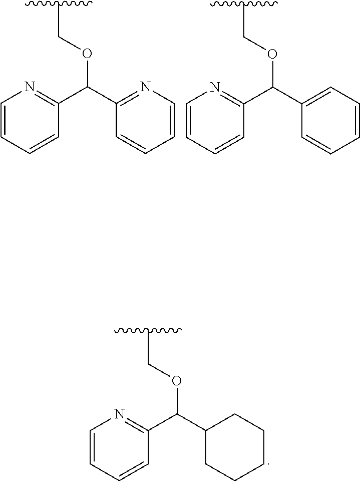

The term “non-aromatic heterocyclylalkyloxyalkyl” means an alkyloxyalkyl substituted with one or more “non-aromatic heterocyclyl” described above. The “non-aromatic heterocyclylalkyloxyalkyl” also includes “non-aromatic heterocyclylalkyloxyalkyl” wherein the alkyl part bonded to the non-aromatic heterocycle is substituted with the above “aromatic carbocyclyl”, “non-aromatic carbocyclyl” and/or “aromatic heterocyclyl”. Examples include tetrahydropyranylmethyloxymethyl, morpholinylmethyloxymethyl, morpholinylethyloxymethyl, piperidinylmethyloxymethyl, piperazinylmethyloxymethyl, and groups of the following formulae with either the R or S stereochemistry or the racemate:

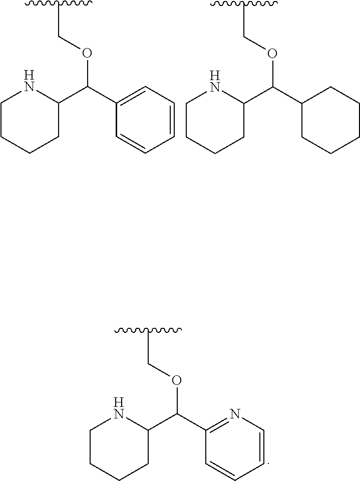

Preferred embodiments of ring A, ring B, R¹, m, R², R^{3a}, R^{3b}, R^{3c}, R^{3d}, ring C, X, Y, R⁴, R^{4C}, R^{4N}, p, q, R⁵, R⁶, R⁷, R⁸, R⁹, R^{5C}, R^{6C}, R^{7C}, R^{8C} and R^{9C} are in the compound represented by formula (I) are described below. A compound having any possible combination of those described below is preferable.

In formula (I), a group represented by formula:

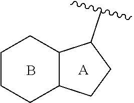

is a group represented by formula:

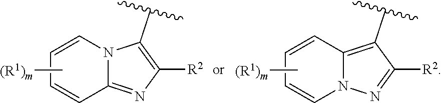

R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted alkylsulfanyl, substituted or unsubstituted alkenylsulfanyl, substituted or unsubstituted alkynylsulfanyl, substituted or unsubstituted alkylsulfinyl, substituted or unsubstituted alkenylsulfinyl, substituted or unsubstituted alkynylsulfinyl, substituted or unsubstituted alkylsulfonyl, substituted or unsubstituted alkenylsulfonyl or substituted or unsubstituted alkynylsulfonyl.

Preferably, R¹ is each independently halogen, cyano, substituted or unsubstituted alkyl or substituted or unsubstituted alkyloxy.

Further preferably, R¹ is each independently halogen, substituted or unsubstituted alkyl or substituted or unsubstituted alkyloxy.

In the case that a group represented by formula:

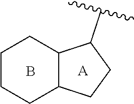

in formula (I) is a group represented as follows:

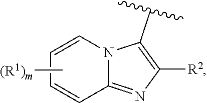

preferably, R¹ is each independently halogen or substituted or unsubstituted alkyl.

Further preferably, R¹ is each independently halogen.

In the case that a group represented by formula:

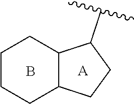

in formula (I) is a group represented as follows:

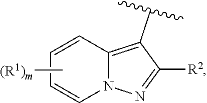

preferably, R¹ is each independently substituted or unsubstituted alkyl or substituted or unsubstituted alkyloxy.

When R¹ is a substituted group, a preferable substituent on said substituted group is selected from halogen, hydroxy, amino, cyano, alkyloxy, alkylamino and the like.

When R¹ is a substituted group, a further preferable substituent on said substituted group is selected from halogen and the like.

m is 0, 1, 2, 3 or 4. Preferably, m is 0, 1 or 2. Further preferably, m is 1 or 2. Particularly preferably, m is 1.

In the case that m is 1, in formula (I), a group represented by formula:

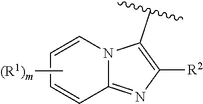

is preferably

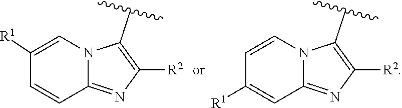

In the case that m is 1, in formula (I), a group represented by formula:

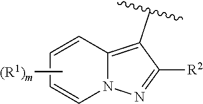

is preferably

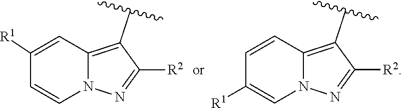

In the case that m is 2, in formula (I), a group represented by formula:

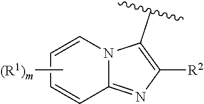

is preferably

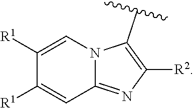

In the case that m is 2, in formula (I), a group represented by formula:

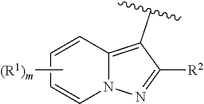

is preferably

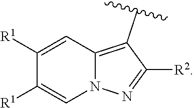

R² is a hydrogen atom, halogen, cyano, substituted or unsubstituted alkyl, substituted or unsubstituted alkenyl or substituted or unsubstituted alkynyl.

Preferably, R² is halogen or substituted or unsubstituted alkyl.

Further preferably, R² is substituted or unsubstituted alkyl.

When R² is a substituted group, a preferable substituent on said substituted group is selected from halogen, hydroxy, amino, cyano, alkyloxy, alkylamino and the like.

When R² is a substituted group, a further preferable substituent on said substituted group is selected from halogen and the like.

R^{3a}, R^{3b}, R^{3c} and R^{3d} are each independently hydrogen atom, halogen, hydroxy, cyano, substituted or unsubstituted alkyl, substituted or unsubstituted alkenyl or substituted or unsubstituted alkynyl, with the proviso that R^{3a}, R^{3b}, R^{3c} and R^{3d} are not simultaneously hydrogen atom.

Preferably, R^{3a} is a hydrogen atom or halogen. Further preferably, R^{3a} is a hydrogen atom.

Preferably, R^{3b} is a hydrogen atom or halogen. Further preferably, R^{3b} is halogen.

Preferably, R^{3c} is a hydrogen atom or halogen. Further preferably, R^{3c} is a hydrogen atom.

Preferably, R^{3d} is a hydrogen atom or halogen. Further preferably, R^{3d} is a hydrogen atom.

Embodiments in which R^{3b} is halogen, and R^{3a}, R^{3c} and R^{3d} are hydrogen atom are also preferred.

Embodiments in which R^{3a} is halogen, and R^{3b}, R^{3c} and R^{3d} are hydrogen atom are also preferred.

Embodiments in which R^{3b} and R^{3c} are each independently halogen, and R^{3a} and R^{3d} are hydrogen atom are also preferred.

Embodiments in which R^{3a} and R^{3b} are each independently halogen, and R^{3c} and R^{3d} are hydrogen atom are also preferred.

Embodiments in which R^{3a} and R^{3c} are each independently halogen, and R^{3b} and R^{3d} are hydrogen atom are also preferred.

When R^{3a} is a substituted group, a preferable substituent on said substituted group is selected from halogen, hydroxy, alkyloxy and the like.

When R^{3b} is a substituted group, a preferable substituent on said substituted group is selected from halogen, hydroxy, alkyloxy and the like.

When R^{3c} is a substituted group, a preferable substituent on said substituted group is selected from halogen, hydroxy, alkyloxy and the like.

When R^{3d} is a substituted group, a preferable substituent on said substituted group is selected from halogen, hydroxy, alkyloxy and the like.

ring C is represented as follows:

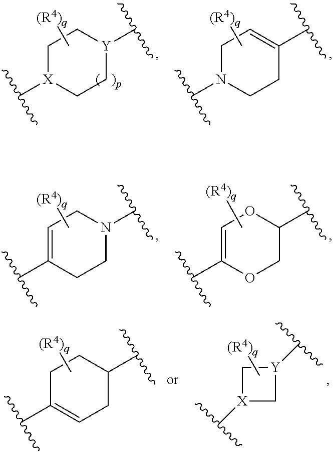

wherein the left bond binds to a group represented by the following formula:

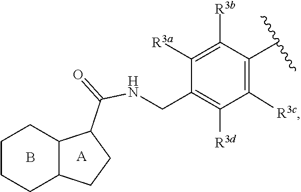

and the right bond binds to a group represented by the following formula:

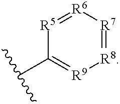

Preferably, ring C is represented as follows:

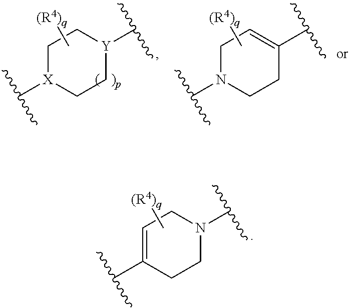

Further preferably, ring C is represented as follows:

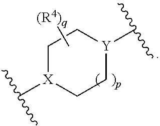

Particularly preferably, ring C is represented as follows:

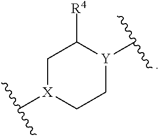

Most preferably, ring C is represented as follows:

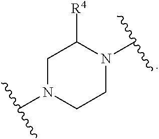

Embodiments in which ring C is represented as follows:

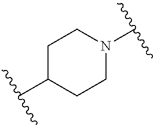 [SMILES: *C1CCN(*)CC1]

are also preferred. Embodiments in which ring C is represented as follows

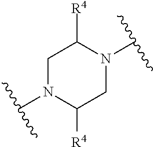

are also preferred.

X is CH or N.

Y is CH or N.

Embodiments in which X is N, and Y is N are also preferred.

Embodiments in which X is N, and Y is CH are also preferred.

Embodiments in which X is CH, and Y is N are also preferred.

R⁴ is each independently halogen, hydroxy, cyano, substituted or unsubstituted alkyl, substituted or unsubstituted alkenyl or substituted or unsubstituted alkynyl;

two R⁴ groups attached to adjacent carbon atoms may be taken together with the carbon atoms to which they are attached to form a substituted or unsubstituted 5- to 6-membered non-aromatic carbocycle or a substituted or unsubstituted 5- to 6-membered non-aromatic heterocycle;

two R⁴ groups attached to a same carbon atom may be taken together with the carbon atom to which they are attached to form a substituted or unsubstituted 3- to 6-membered non-aromatic carbocycle or a substituted or unsubstituted 3- to 6-membered non-aromatic heterocycle;

two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N}.

Preferably, R⁴ is each independently halogen or substituted or unsubstituted alkyl.

Further preferably, R⁴ is each independently substituted or unsubstituted alkyl.

Embodiments in which two R⁴ groups are taken together to form (C2-C4) bridge are also preferred. For example, it includes the following:

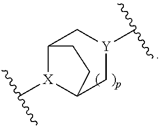

Embodiments in which two R⁴ groups attached to adjacent carbon atoms are taken together with the carbon atoms to which they are attached to form a substituted or unsubstituted 5- to 6-membered non-aromatic carbocycle are also preferred. For example, it includes the following (as cis or trans fused isomers either racemic or stereodefined):

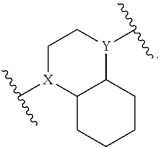

Embodiments in which two R⁴ groups attached to a same carbon atom are taken together with the carbon atom to which they are attached to form a substituted or unsubstituted 3- to 6-membered non-aromatic carbocycle are also preferred. For example it includes the following:

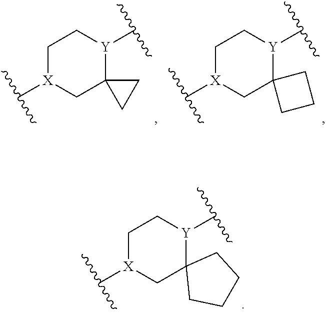

When R⁴ is a substituted group, a preferable substituent on said substituted group is selected from halogen, hydroxy, amino, cyano, alkyloxy, alkylamino and the like.

R^{4C} is each independently a hydrogen atom, halogen, hydroxy, cyano or substituted or unsubstituted alkyl.

Preferably, R^{4C} is each independently a hydrogen atom, halogen or substituted or unsubstituted alkyl.

Further preferably, R^{4C} is each independently a hydrogen atom.

When R^{4C} is a substituted group, a preferable substituent on said substituted group is selected from halogen, hydroxy, amino, alkyloxy, alkylamino and the like.

R^{4N} is each independently a hydrogen atom or substituted or unsubstituted alkyl.

Preferably, R^{4N} is each independently substituted or unsubstituted alkyl.

When R^{4N} is a substituted group, a preferable substituent on said substituted group is selected from halogen and the like.

p is 0 or 1. Preferably, p is 1.

q is 0, 1, 2, 3 or 4. Preferably, q is 0, 1 or 2. Further preferably, q is 1 or 2. Particularly preferably, q is 1.

R⁵ is CR^{5C} or N; R⁶ is CR^{6C} or N; R⁷ is CR^{7C} or N; R⁸ is CR^{8C} or N; R⁹ is CR^{9C} or N; with the proviso that R⁵, R⁶, R⁷, R⁸ and R⁹ are not simultaneously N.

Preferably, R⁵ is CR⁵c.

Preferably, R⁶ is CR⁶c.

Preferably, R⁷ is CR^{C}.

Preferably, R⁸ is CR^{8C}.

Preferably, R⁹ is CR^{9C}.

R^{5C}, R^{6C}, R^{7C}, R^{8C} and R^{9C} are each independently hydrogen atom, halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted aromatic carbocyclyloxy, substituted or unsubstituted non-aromatic carbocyclyloxy, substituted or unsubstituted aromatic heterocyclyloxy, substituted or unsubstituted non-aromatic heterocyclyloxy or pentafluorothio.

Preferably, R^{5C} and R^{9C} are each independently hydrogen atom or halogen. Further preferably, R^{5C} and R^{9C} are hydrogen atom.

When R^{5C} is a substituted group, a preferable substituent on said substituted group is selected from halogen and the like.

When R^{9C} is a substituted group, a preferable substituent on said substituted group is selected from halogen and the like.

Preferably, R^{6C} and R^{8C} are each independently hydrogen atom, halogen, substituted or unsubstituted alkyl or substituted or unsubstituted alkyloxy.

Further preferably, R^{6C} and R^{8C} are each independently hydrogen atom, halogen or substituted or unsubstituted alkyloxy.

Particularly preferably, R^{6C} and R^{8C} are each independently hydrogen atom or substituted or unsubstituted alkyloxy.

Most preferably, R^{6C} and R^{8C} are hydrogen atom.

When R^{6C} is a substituted group, a preferable substituent on said substituted group is selected from halogen and the like.

When R^{8C} is a substituted group, a preferable substituent on said substituted group is selected from halogen and the like.

Preferably, R^{7C} is a hydrogen atom, halogen, substituted or unsubstituted alkyl, substituted or unsubstituted alkyloxy or substituted or unsubstituted non-aromatic carbocyclyloxy.

Further preferably, R^{7C} is halogen, substituted or unsubstituted alkyl or substituted or unsubstituted alkyloxy.

Particularly preferably, R^{7C} is substituted or unsubstituted alkyl or substituted or unsubstituted alkyloxy.

Most preferably, R^{7C} is substituted or unsubstituted alkyloxy, including trihaloalkyloxy (like OCF₃).

When R^{7C} is a substituted group, a preferable substituent on said substituted group is selected from halogen, hydroxy, amino, alkyloxy, alkylamino, non-aromatic carbocyclyl and the like.

When R^{7C} is a substituted group, a further preferable substituent on said substituted group is selected from halogen and the like.

Preferred combinations of substituents of a compound represented by formula (I) include the following 1) to 6):

1) a compound wherein a group represented by formula:

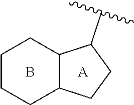

is a group represented as follows:

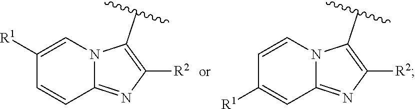

R¹ is each independently halogen or substituted or unsubstituted alkyl; R² is each independently halogen or substituted or unsubstituted alkyl; R^{3b} is halogen; R^{3a}, R^{3c} and R^{3d} are hydrogen atom; ring C is represented as follows with either the R or S stereochemistry or the racemate:

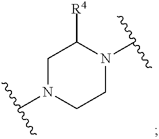

R⁴ is substituted or unsubstituted alkyl; R⁵, R⁶, R⁸ and R⁹ are CH; R⁷ is CR^{7C}; R^{7C} is substituted or unsubstituted alkyloxy, including trihaloalkyloxy (like OCF₃):

2) a compound wherein a group represented by formula:

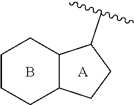

is a group represented as follows:

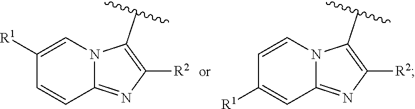

R¹ is each independently halogen; R² is each independently substituted or unsubstituted alkyl; R^{3b} is halogen; R^{3a}, R^{3c} and R^{3d} are hydrogen atom; ring C is represented as follows with either the R or S stereochemistry or the racemate:

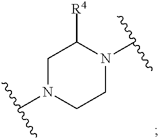

R⁴ is substituted or unsubstituted alkyl; R⁵, R⁶, R⁸ and R⁹ are CH; R⁷ is CR^{7C}; R^{7C} is substituted or unsubstituted alkyloxy, including trihaloalkyloxy (like OCF₃):

3) a compound wherein a group represented by formula:

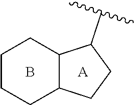

is a group represented as follows:

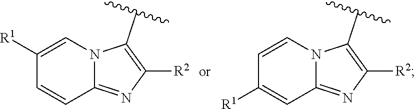

R¹ is each independently halogen; R² is each independently substituted or unsubstituted alkyl; R^{3a} is halogen; R^{3b}, R^{3c} and R^{3d} are hydrogen atom; ring C is represented as follows:

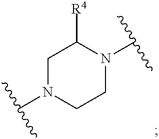

R⁵, R⁶, R⁸ and R⁹ are CH; R⁷ is CR^{7C}; R^{7C} is substituted or unsubstituted alkyl:

4) a compound wherein a group represented by formula:

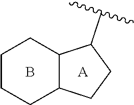

is a group represented as follows:

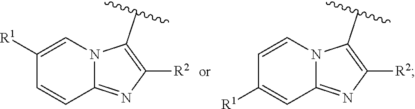

R¹ is each independently halogen; R² is each independently substituted or unsubstituted alkyl; R^{3b} is halogen; R^{3a}, R^{3c} and R^{3d} are hydrogen atom; ring C is represented as follows with either the R or S stereochemistry or the racemate:

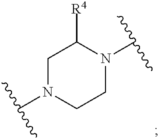

R⁴ is substituted or unsubstituted alkyl; R⁵, R⁶, R⁸ and R⁹ are CH; R⁷ is CR^{7C}; R^{7C} is substituted or unsubstituted alkyloxy, including trihaloalkyloxy (like OCF₃):

5) a compound wherein a group represented by formula:

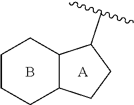

is a group represented as follows:

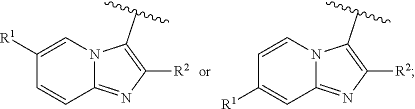

R¹ is each independently substituted or unsubstituted alkyl; R² is each independently substituted or unsubstituted alkyl; R^{3b} is halogen; R^{3a}, R^{3c} and R^{3d} are hydrogen atom; ring C is represented as follows with either the R or S stereochemistry or the racemate:

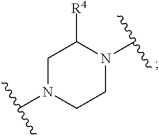

R⁴ is substituted or unsubstituted alkyl; R⁵, R⁶, R⁸ and R⁹ are CH; R⁷ is CR^{7C}; R^{7C} is substituted or unsubstituted alkyloxy, including trihaloalkyloxy (like OCF₃):

6) a compound wherein Ring A and ring B are represented as follows:

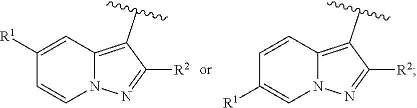

R¹ is each independently substituted or unsubstituted alkyl or substituted or unsubstituted alkyloxy; R² is each independently halogen or substituted or unsubstituted alkyl; R^{3b} is halogen; R^{3a}, R^{3c} and R^{3d} are hydrogen atom; ring C is represented as follows:

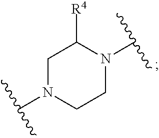

R⁴ is substituted or unsubstituted alkyl; R⁵, R⁶, R⁸ and R⁹ are CH; R⁷ is CR^{7C}; R^{7C} is substituted or unsubstituted alkyloxy, including trihaloalkyloxy (like OCF₃).

The compounds of formula (I) are not limited to specific isomers but include all possible isomers (e.g., keto-enol isomers, imine-enamine isomers, diastereoisomers, enantiomers, or rotamers), racemates or mixtures thereof.

One or more hydrogen, carbon and/or other atom(s) in the compounds of formula (I) may be replaced with isotopes of hydrogen, carbon and/or other atoms respectively. Examples of isotopes include hydrogen, carbon, nitrogen, oxygen, phosphorus, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S ¹⁸F, ¹²³I and ³⁶Cl respectively. The compounds of formula (I) include the compounds replaced with these isotopes. The compounds replaced with the above isotopes are useful as medicines and include all of radiolabeled compounds of the compound of formula (I). A “method of radiolabeling” in the manufacture of the “radiolabeled compounds” is encompassed by the present invention, and the “radiolabeled compounds” are useful for studies on metabolized drug pharmacokinetics, studies on binding assay and/or diagnostic tools.

A radiolabeled compound of the compounds of formula (I) can be prepared using well-known methods in this field of the invention. For example, a tritium-labeled compound of formula (I) can be prepared by introducing a tritium to a certain compound of formula (I) through a catalytic dehalogenation reaction using a tritium. This method comprises reacting an appropriately-halogenated precursor of the compound of formula (I) with tritium gas in the presence of an appropriate catalyst, such as Pd/C, and in the presence or absent of a base. The other appropriate method of preparing a tritium-labeled compound can be referred to “Isotopes in the Physical and Biomedical Sciences, Vol. 1, Labeled Compounds (Part A), Chapter 6 (1987)”. A ¹⁴C-labeled compound can be prepared by using a raw material having ¹⁴C.

The pharmaceutically acceptable salts of the compounds of Formula (I) include, for example, salts with alkaline metal (e.g., lithium, sodium, or potassium), alkaline earth metal (e.g., calcium or barium), magnesium, transition metal (e.g., zinc or iron), ammonia, organic bases (e.g., trimethylamine, triethylamine, dicyclohexylamine, ethanolamine, diethanolamine, triethanolamine, meglumine, diethanolamine, ethylenediamine, pyridine, picoline, or quinoline), amino acids, or salts with inorganic acids (e.g., hydrochloric acid, sulfuric acid, nitric acid, carbonic acid, hydrobromic acid, phosphoric acid, or hydroiodic acid) or organic acids (e.g., formic acid, acetic acid, propionic acid, trifluoroacetic acid, citric acid, lactic acid, tartaric acid, oxalic acid, maleic acid, fumaric acid, succinic acid, mandelic acid, glutaric acid, malic acid, benzoic acid, phthalic acid, ascorbic acid, benzenesulfonic acid, p-toluenesulfonic acid, methanesulfonic acid, or ethanesulfonic acid). These salts can be formed by the usual methods.

The compounds of formula (I) or pharmaceutically acceptable salts thereof may form solvates (e.g., hydrates), co-crystal and/or crystal polymorphs. The present invention encompasses those various solvates, co-crystal and crystal polymorphs. “Solvates” may be those wherein any numbers of solvent molecules (e.g., water molecules) are coordinated with the compounds of formula (I). When the compounds of formula (I) or pharmaceutically acceptable salts thereof are allowed to stand in the atmosphere, the compounds may absorb water, resulting in attachment of adsorbed water or formation of hydrates. Recrystallization of the compounds of formula (I) or pharmaceutically acceptable salts thereof may produce crystal polymorphs. “Co-crystal” means that a compound of formula (I) or a salt thereof and a counter-molecule exist in the same crystal lattice, and it can be formed with any number of counter-molecules.

The compounds of formula (I) of the present invention or pharmaceutically acceptable salts thereof may form prodrugs. The present invention also encompasses such various prodrugs. Prodrugs are derivatives of the compounds of the present invention that have chemically or metabolically degradable groups, and compounds that are converted to the pharmaceutically active compounds of the present invention through solvolysis or under physiological conditions in vivo. Prodrugs include compounds that are converted to the compounds of formula (I) through enzymatic oxidation, reduction, hydrolysis or the like under physiological conditions in vivo, compounds that are converted to the compounds of formula (I) through hydrolysis by gastric acid etc., and the like. Methods for selecting and preparing suitable prodrug derivatives are described in, for example, “Design of Prodrugs, Elsevier, Amsterdam, 1985”. Prodrugs themselves may have some activity.

When the compounds of formula (I) or pharmaceutically acceptable salts thereof have hydroxyl group(s), prodrugs include acyloxy derivatives and sulfonyloxy derivatives that are prepared by, for example, reacting compounds having hydroxyl group(s) with suitable acyl halide, suitable acid anhydride, suitable sulfonyl chloride, suitable sulfonyl anhydride and mixed anhydride, or with a condensing agent. For example, they include CH₃COO⁻, C₂H₅COO⁻, tert-BuCOO⁻, C₁₅H₃₁COO⁻, PhCOO⁻, (m-NaOOCPh)COO⁻, NaOOCCH₂CH₂COO⁻, CH₃CH(NH₂)COO⁻, CH₂N(CH₃)₂COO⁻, CH₃SO₃⁻, CH₃CH₂SO₃⁻, CF₃SO₃⁻, CH₂FSO₃⁻, CF₃CH₂SO₃⁻, p-CH₃O⁻, PhSO₃⁻, PhSO₃⁻ and p-CH₃PhSO₃⁻.

The term “pharmaceutically acceptable” means preventively or therapeutically harmless.

General procedures for the synthesis of the compounds of the present invention are described below. Starting materials and reaction reagents used in such synthesis are commercially available or can be synthesized according to methods well known in the art using compounds commercially available. Further, extraction, purification and the like may be performed in accordance with the methods carried out in the art.

In the following all steps, when a substituent which impedes a reaction, e.g. hydroxy, mercapto, amino, formyl, carbonyl, carboxy, is possessed, the substituent is protected by the method described in Protective Groups in organic Synthesis, and Theodora W Greene (John Wiley & Sons, hereinafter referred to as literature A) in advance, and the protecting group may be removed at a desirable stage. In addition, in the all steps, an order of steps to be implemented may be appropriately changed, and each intermediate may be isolated, and used in a next step. All of reaction time, reaction temperature, solvents, reagents, protecting groups, etc. are mere exemplification and not limited as long as they do not cause an adverse effect on a reaction.

For example, the compounds represented by Formula (I) of the present invention can be produced according to general procedures as described below. Also, the compounds of the invention can be prepared according to other methods based on the knowledge in organic chemistry.

### Preparation of Compound a3

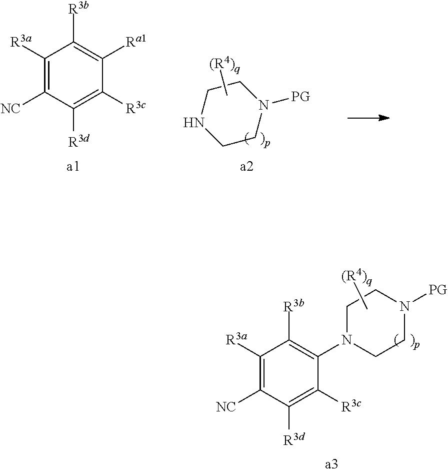

wherein

PG is an amino protecting group such as Boc, Cbz and etc., R^{a1} is halogen, triflate, nonaflate, mesylate or tosylate, the other symbols are as defined above.

The compound a1 is reacted with a2 in the presence of the base to obtain Compound a3.

Examples of the solvent include toluene, DMF, DMA, tetrahydrofuran, ethanol, water, toluene, acetonitrile, 1,4-dioxane and the like, and these solvents may be used alone or in combination.

Examples of the base include potassium tert-butoxide, sodium hydride, potassium carbonate, cesium carbonate, triethylamine, diisopropylethylamine, DBU and the like. The amount of the base may be 1 to 10 mole equivalents, preferably 1 to 5 mole equivalents of Compound a1.

The reaction temperature may be room temperature to 200° C., preferably 50° C. to 150° C., and the reaction can be conducted in seeled tube as required.

The reaction time may be 0.1 to 24 hours, preferably 1 to 12 hours.

### Preparation of Compound a5

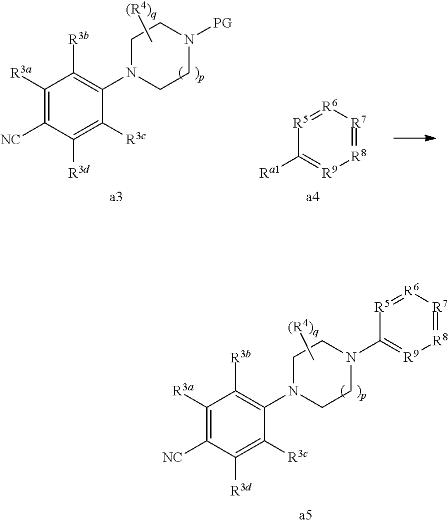

wherein each symbol is as defined above.

Compound a5 can be obtained by reacting with a4 in the presence of Palladium, Ligand and base after reacting Compound a3 with deprotecting agents.

Examples of the reaction solvent for deprotecting reaction include ethyl acetate, water, dicloromethane, N, N-dimethylformamide, ethanol, tetrahydrofuran, methanol, 1,4-dioxane, acetonitrile, toluene and the like, and these solvents may be used alone or in combination.

Examples of the deprotecting agent include hydrochloric acid/ethyl acetate, methane sulfonic acid, trifluoroacetic acid, sulfuric acid, iodotrimethylsilane, aluminium trichloride, bromocatechol borane, trimethylsilyl chloride, trimethylsilyl triflate and the like. The amount of the deprotecting agent may be 1 to 100 mole equivalents, preferably 1 to 50 mole equivalents of Compound a4.

Examples of the Palladium include palladium acetate, Pd(PPh₃)₄, PdCl₂(PPh₃)₂, Pd₂(dba)₃ and the like. The amount of the Palladium may be 0.01 to 5 mole equivalents, preferably 0.01 to 1 mole equivalents of Compound a4.

Examples of the Ligand include 2-Dicyclohexylphosphino-2′,6′-diisopropoxy-1,1′-biphenyl, Dicyclohexyl-[2-(2,4,6-triisopropylphenyl)phenyl]phosphane, (9,9-dimethyl-9H-xanthene-4,5-diyl)bis(diphenylphosphane) and the like. The amount of the Ligand may be 0.01 to 5 mole equivalents, preferably 0.01 to 1 mole equivalents of Compound a3.

Examples of the base include cesium carbonate, potassium carbonate, sodium carbonate, potassium tert-butoxide, sodium tert-butoxide and the like. The amount of the base may be 1 to 10 mole equivalents, preferably 1 to 5 mole equivalents of Compound a4.

Examples of the reaction solvent include methanol, N,N-dimethylformamide, N, N-dimethylacetamide, tetrahydrofuran, 1,4-dioxane, acetonitrile, toluene, ethyl acetate and the like, and these solvents may be used alone or in combination.

The reaction temperature may be room temperature to 200° C., preferably 50° C. to 150° C., and the reaction can be conducted in seeled tube as required.

The reaction time may be 0.1 to 24 hours, preferably 1 to 12 hours.

### Preparation of Compound a6

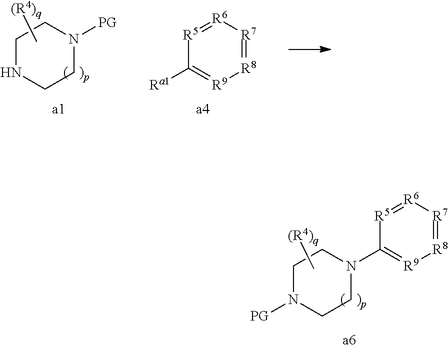

wherein each symbol is as defined above.

Compound a6 can be obtained by reacting with a1 and a4 in the presence of Palladium, Ligand and base.

Examples of the Palladium include palladium acetate, Pd(PPh₃)₄, PdCl₂(PPh₃)₂, Pd₂(dba)₃ and the like. The amount of the Palladium may be 0.01 to 5 mole equivalents, preferably 0.01 to 1 mole equivalents of Compound a1.

Examples of the Ligand include 2-dicyclohexylphosphino-2′,6′-diisopropoxy-1,1′-biphenyl, dicyclohexyl-[2-(2,4,6-triisopropylphenyl)phenyl]phosphane, (9,9-dimethyl-9H-xanthene-4,5-diyl)bis(diphenylphosphane) and the like. The amount of the Ligand may be 0.01 to 5 mole equivalents, preferably 0.01 to 1 mole equivalents of Compound a1.

Examples of the base include cesium carbonate, potassium carbonate, sodium carbonate, potassium tert-butoxide, sodium tert-butoxide and the like. The amount of the base may be 1 to 10 mole equivalents, preferably 1 to 5 mole equivalents of Compound a1.

Examples of the reaction solvent include methanol, N,N-dimethylformamide, N, N-dimethylacetamide, tetrahydrofuran, 1,4-dioxane, acetonitrile, toluene, ethyl acetate and the like, and these solvents may be used alone or in combination.

The reaction temperature may be room temperature to 200° C., preferably 50° C. to 150° C., and the reaction can be conducted in seeled tube as required.

The reaction time may be 0.1 to 24 hours, preferably 1 to 12 hours.

### Preparation of Compound a5

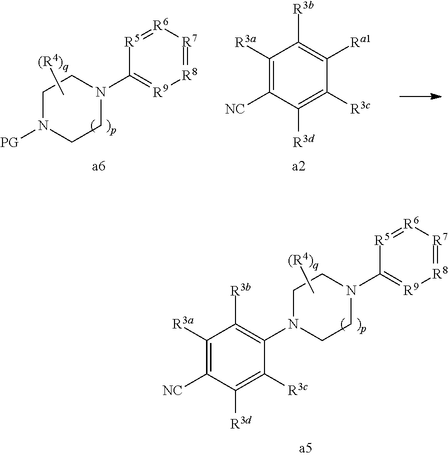

wherein each symbol is as defined above.

Compound a5 can be obtained by reacting with a2 in the presence of the base after reacting Compound a6 with deprotecting agents.

Examples of the reaction solvent for deprotecting reaction include ethyl acetate, water, dicloromethane, N, N-dimethylformamide, ethanol, tetrahydrofuran, methanol, 1,4-dioxane, acetonitrile, toluene and the like, and these solvents may be used alone or in combination.

Examples of the deprotecting agent include hydrochloric acid/ethyl acetate, methane sulfonic acid, trifluoroacetic acid, sulfuric acid, iodotrimethylsilane, aluminium trichloride, bromocatechol borane, trimethylsilyl chloride, trimethylsilyl triflate and the like. The amount of the deprotecting agent may be 1 to 100 mole equivalents, preferably 1 to 50 mole equivalents of Compound a2.

Examples of the solvent include toluene, DMF, DMA, dimethyl sulfoxide tetrahydrofuran, ethanol, water, toluene, acetonitrile, 1,4-dioxane and the like, and these solvents may be used alone or in combination.

Examples of the base include potassium tert-butoxide, sodium hydride, potassium carbonate, cesium carbonate, triethylamine, diisopropylethylamine, DBU and the like. The amount of the base may be 1 to 10 mole equivalents, preferably 1 to 5 mole equivalents of Compound a2.

The reaction temperature may be room temperature to 200° C., preferably 50° C. to 150° C., and the reaction can be conducted in seeled tube as required.

The reaction time may be 0.1 to 24 hours, preferably 1 to 12 hours.

### Preparation of Compound a7

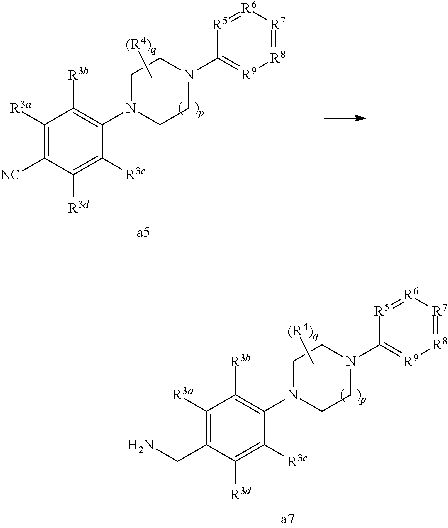

wherein each symbol is as defined above.

Compound a7 can be obtained by reacting Compound a5 with reductants.

Examples of the reaction solvent include ethyl acetate, water, dicloromethane, N, N-dimethylformamide, ethanol, tetrahydrofuran, methanol, acetic acid, 1,4-dioxane, acetonitrile, toluene and the like, and these solvents may be used alone or in combination.

Examples of the reductants include hydrogen with Pd carbon, hydrogen with Pd(OH)₂, sodium borohydrodie with NiCl₂(H₂O)₆, lithium aluminumhydride and the like. The amount of the reductants may be 1 to 100 mole equivalents, preferably 1 to 10 mole equivalents of Compound a5.

The reaction temperature may be 0° C. to 200° C., preferably 0° C. to 100° C.

The reaction time may be 0.1 to 24 hours, preferably 1 to 12 hours.

### Preparation of Compound a9

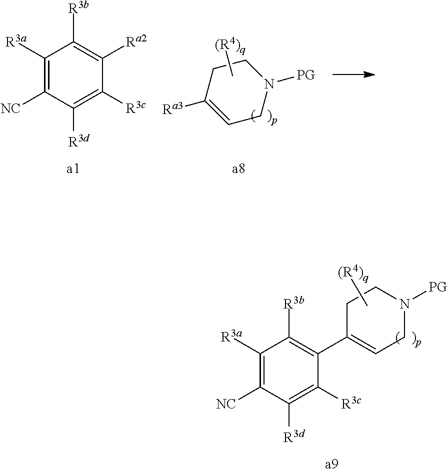

wherein

R^{a2} is halogen, triflate, nonaflate, mesylate, tosylate, bornic acid or boronate, R^{a3} is triflate, nonaflate, mesylate, tosylate, bornic acid or boronate, the other symbols are as defined above.

The compound a1 is reacted with a8 in the presence of the Palladium and base to obtain Compound a9.

Examples of the Palladium include palladium acetate, Pd(PPh₃)₄, PdCl₂(PPh₃)₂, Pd₂(dba)₃, PdCl₂(dppf) and the like. The amount of the Palladium may be 0.01 to 5 mole equivalents, preferably 0.01 to 1 mole equivalents of Compound a1.

Examples of the base include cesium carbonate, potassium carbonate, sodium carbonate and the like. The amount of the base may be 1 to 10 mole equivalents, preferably 1 to 5 mole equivalents of Compound a1.

Examples of the reaction solvent include water, DMF, DMA, tetrahydrofuran, 1,4-dioxane, acetonitrile, toluene, ethyl acetate and the like, and these solvents may be used alone or in combination.

The reaction temperature may be room temperature to 200° C., preferably 50° C. to 150° C., and the reaction can be conducted in seeled tube as required.

The reaction time may be 0.1 to 24 hours, preferably 1 to 12 hours.

### Preparation of Compound a10

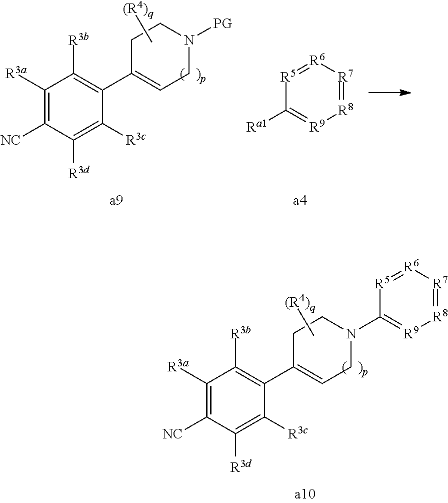

wherein each symbol is as defined above.

Compound a10 can be obtained by reacting with Compound a9 in the presence of Palladium, ligand and base after reacting Compound a4 with deprotecting agents.

Examples of the reaction solvent for deprotecting reaction include ethyl acetate, water, dicloromethane, N, N-dimethylformamide, ethanol, tetrahydrofuran, methanol, 1,4-dioxane, acetonitrile, toluene and the like, and these solvents may be used alone or in combination.

Examples of the deprotecting agent include hydrochloric acid/ethyl acetate, methane sulfonic acid, trifluoroacetic acid, sulfuric acid, iodotrimethylsilane, aluminium trichloride, bromocatechol borane, trimethylsilyl chloride, trimethylsilyl triflate and the like. The amount of the deprotecting agent may be 1 to 100 mole equivalents, preferably 1 to 50 mole equivalents of Compound a4.

Examples of the Palladium include palladium acetate, Pd(PPh₃)₄, PdCl₂(PPh₃)₂, Pd₂(dba)₃ and the like. The amount of the Palladium may be 0.01 to 5 mole equivalents, preferably 0.01 to 1 mole equivalents of Compound a4.

Examples of the Ligand include 2-dicyclohexylphosphino-2′,6′-diisopropoxy-1,1′-biphenyl, dicyclohexyl-[2-(2,4,6-triisopropylphenyl)phenyl]phosphane, (9,9-dimethyl-9H-xanthene-4,5-diyl)bis(diphenylphosphane) and the like. The amount of the Ligand may be 0.01 to 5 mole equivalents, preferably 0.01 to 1 mole equivalents of Compound a4.

Examples of the base include cesium carbonate, potassium carbonate, sodium carbonate, potassium tert-butoxide, sodium tert-butoxide and the like. The amount of the base may be 1 to 10 mole equivalents, preferably 1 to 5 mole equivalents of Compound a4.

Examples of the reaction solvent include methanol, N,N-dimethylformamide, N, N-dimethylacetamide, tetrahydrofuran, 1,4-dioxane, acetonitrile, toluene, ethyl acetate and the like, and these solvents may be used alone or in combination.

The reaction temperature may be room temperature to 200° C., preferably 50° C. to 150° C., and the reaction can be conducted in seeled tube as required.

The reaction time may be 0.1 to 24 hours, preferably 1 to 12 hours.

### Preparation of Compound a11

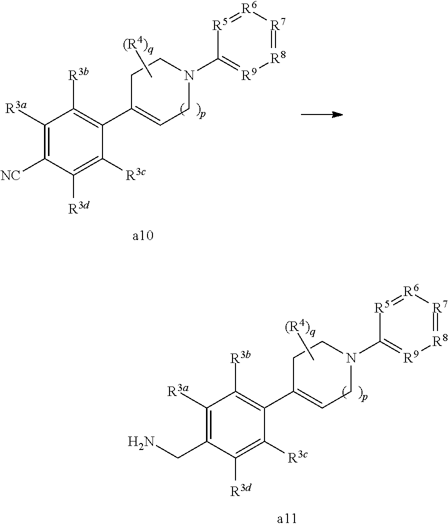

wherein each symbol is as defined above.

Compound a11 can be obtained by reacting Compound a10 with reductants.

Examples of the reaction solvent include ethyl acetate, water, dicloromethane, N, N-dimethylformamide, ethanol, tetrahydrofuran, methanol, acetic acid, 1,4-dioxane, acetonitrile, toluene and the like, and these solvents may be used alone or in combination.

Examples of the reductants include sodium borohydrodie with NiCl₂(H₂O)₆, lithium aluminumhydride, borane tetrahydrofran, borane dimethylsulfide and the like. The amount of the reductants may be 1 to 100 mole equivalents, preferably 1 to 10 mole equivalents of Compound a10.

The reaction temperature may be 0° C. to 200° C., preferably 0° C. to 100° C.

The reaction time may be 0.1 to 24 hours, preferably 1 to 12 hours.

### Preparation of Compound a13

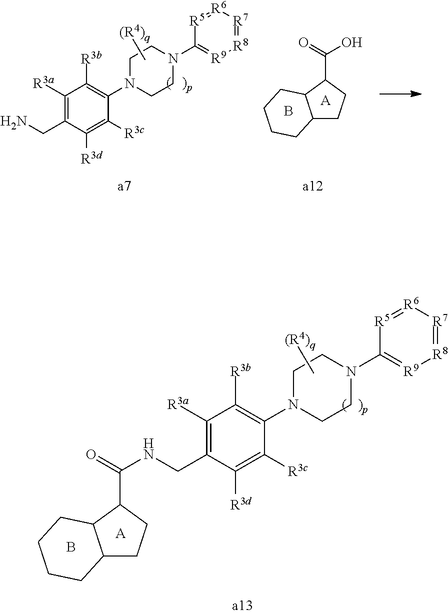

wherein each symbol is as defined above.

Compound a13 can be obtained by reacting Compound a7 with carbocylic acid (Compound a12) in the presence of condensing agents and base.

Examples of the reaction solvent include N,N-dimethylformamide, ethanol, water, dichloromethane, tetrahydrofuran, methanol, 1,4-dioxane, acetonitrile, toluene, ethyl acetate and the like, and these solvents may be used alone or in combination.

Examples of the base include triethylamine, potassium tert-butoxide, potassium carbonate, cesium carbonate, diisopropylethylamine, DBU and the like. The amount of the base may be 1 to 10 mole equivalents, preferably 1 to 5 mole equivalents of Compound a12.

Examples of the condensing agent include HATU, WSC, DCC, HOBt and the like. The amount of the condensing agent may be 1 to 10 mole equivalents, preferably 1 to 5 mole equivalents of Compound a12.

The carboxylic acid may be used in 1 to 10 mole equivalents, preferably 1 to 5 mole equivalents of Compound a7.

The reaction temperature may be under ice-cooling to reflux temperature, preferably room temperature.

The reaction time may be 0.1 to 24 hours, preferably 1 to 5 hours.

### Preparation of Compound a14

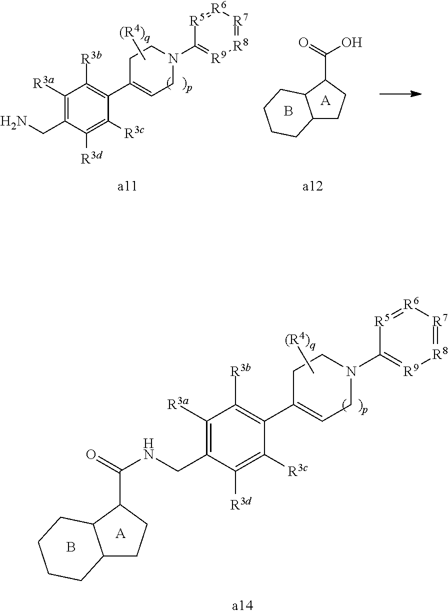

wherein each symbol is as defined above.

Compound a14 can be obtained by reacting Compound a11 with carbocylic acid (Compound a12) in the presence of condensing agents and base. Examples of the reaction solvent include N,N-dimethylformamide, ethanol, water, dichloromethane, tetrahydrofuran, methanol, 1,4-dioxane, acetonitrile, toluene, ethyl acetate and the like, and these solvents may be used alone or in combination.

Examples of the base include triethylamine, potassium tert-butoxide, potassium carbonate, cesium carbonate, diisopropylethylamine, DBU and the like. The amount of the base may be 1 to 10 mole equivalents, preferably 1 to 5 mole equivalents of Compound a12.

Examples of the condensing agent include HATU, WSC, DCC, HOBt and the like. The amount of the condensing agent may be 1 to 10 mole equivalents, preferably 1 to 5 mole equivalents of Compound a12.

The carboxylic acid may be used in 1 to 10 mole equivalents, preferably 1 to 5 mole equivalents of Compound a11.

The reaction temperature may be under ice-cooling to reflux temperature, preferably room temperature.

The reaction time may be 0.1 to 24 hours, preferably 1 to 5 hours.

### Preparation of Compound a15

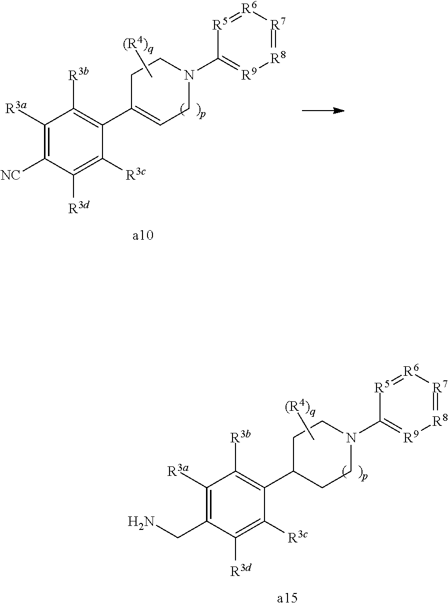

wherein each symbol is as defined above.

Compound a15 can be obtained by reacting Compound a10 with reductants.

Examples of the reaction solvent include ethyl acetate, water, dicloromethane, N, N-dimethylformamide, ethanol, tetrahydrofuran, methanol, acetic acid, 1,4-dioxane, acetonitrile, toluene and the like, and these solvents may be used alone or in combination.

Examples of the reductants include hydrogen with Pd carbon, hydrogen with Pd(OH)₂, and the like. The amount of the reductants may be 1 to 100 mole equivalents, preferably 1 to 10 mole equivalents of Compound a10.

The reaction temperature may be 0° C. to 200° C., preferably 0° C. to 100° C.

The reaction time may be 0.1 to 24 hours, preferably 1 to 12 hours.

### Preparation of Compound a16

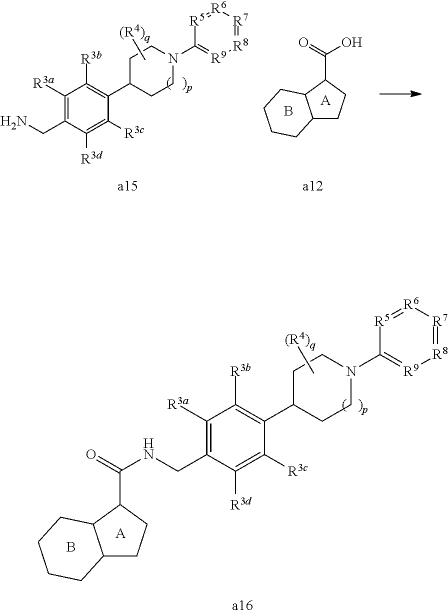

wherein each symbol is as defined above.

Compound a16 can be obtained by reacting Compound a15 with carbocylic acid (Compound a12) in the presence of condensing agents and base.

Examples of the reaction solvent include N,N-dimethylformamide, ethanol, water, dichloromethane, tetrahydrofuran, methanol, 1,4-dioxane, acetonitrile, toluene, ethyl acetate and the like, and these solvents may be used alone or in combination.

Examples of the base include triethylamine, potassium tert-butoxide, potassium carbonate, cesium carbonate, diisopropylethylamine, DBU and the like. The amount of the base may be 1 to 10 mole equivalents, preferably 1 to 5 mole equivalents of Compound a12.

Examples of the condensing agent include HATU, WSC, DCC, HOBt and the like. The amount of the condensing agent may be 1 to 10 mole equivalents, preferably 1 to 5 mole equivalents of Compound a12.

The carboxylic acid may be used in 1 to 10 mole equivalents, preferably 1 to 5 mole equivalents of Compound a15.

The reaction temperature may be under ice-cooling to reflux temperature, preferably room temperature.

The reaction time may be 0.1 to 24 hours, preferably 1 to 5 hours.

The compounds of the present invention are useful in the treatment or prevention of a mycobacterial infection, especially non-tuberculous mycobacterial infection. Such compounds may work by interfering with ATP synthase in pathogenic mycobacteria, with the inhibition of cytochrome bc1 activity as the primary mode of action.

The compounds of the present invention have not only the above described activity but also usefulness as a medicine, and have any or all of the following superior features:

a) The inhibitory activity for CYP enzymes (e.g., CYP1A2, CYP2C9, CYP2C19, CYP2D6, CYP3A4 and the like) is weak.
b) The compound demonstrates good pharmacokinetics, such as a high bioavailability, moderate clearance, high distribution to a targeted tissue and the like.
c) The compound has a high metabolic stability.
d) The compound has no irreversible inhibitory effect against CYP enzymes (e.g., CYP3A4) when the concentration is within the range described in the present description as the measurement conditions.
e) The compound has no mutagenicity.
f) The compound is associated with a low cardiovascular risk.
g) The compound has a high solubility.
h) The compound causes less induction of drug-metabolizing enzyme
i) The compound has less risk of phototoxicity,
j) The compound has less risk of hepatotoxicity,
k) The compound has less risk of kidney toxicity,
l) The compound has less risk of gastrointestinal disorders, and
m) The compound has intense efficacy.

The route of administration of the medicament of the present invention can be administered by either oral or parenteral methods and is not particularly limited to them.

In the case of oral administration, it can be administered by the usual manner in the form of solid preparations for internal use (e.g., tablets, powders, granules, capsules, pills, films), internal solutions (e.g., suspensions, emulsions, elixirs, syrups, limonade agents, alcoholic agents, fragrance solutions, extracts, decoctions, tinctures), and the like. The tablet may be sugar-coated tablets, film-coated tablets, enteric coated tablets, extended release tablets, troches, sublingual tablets, buccal tablets, chewable tablets or orally disintegrating tablets. The powders and granules may be dry syrups. The capsule may be soft capsule, microcapsules or sustained release capsules.

In the case of parenteral administration, any forms of injections, drops, external preparations (e.g., eye drops, nasal drops, ear drops, aerosols, inhalants, lotions, infusions, coating agents, gargles, enemas, ointments, plasters, jellies, creams, patches, cataplasms, external powders, suppositories) which are usually used can be suitably administered. The injection may be emulsions such as O/W, W/O, O/W/O or W/G/W type.

Optionally, the effective amounts of the compound used in the medicament of the present invention may be mixed as necessary with various pharmaceutical additives such as excipients, binders, disintegrants, and/or lubricants suitable for the dosage form to give the pharmaceutical composition. Furthermore, the pharmaceutical composition can be used for children, the elderly, serious patients or surgery, by appropriately changing the effective amount of the compound used in the medicament of the present invention, the dosage form and/or various pharmaceutical additives. The pediatric pharmaceutical composition is preferably administered to patients aged under 12 years old or 15 years old. The pediatric pharmaceutical composition can also be administered to patients less than 4 weeks after birth, 4 weeks to less than 1 year old after birth, 1 year old to less than 7 years old, 7 years old to less than 15 years old, or 15 years old to 18 years old. The pharmaceutical composition for the elderly is preferably administered to patients over 65 years old.

Although the dosage of the pharmaceutical composition of the present invention should be determined in consideration of the patient's age and body weight, the type and degree of diseases, the administration route and the like, a usual oral dosage is 0.05 to 100 and preferably 0.1 to 10 mg/kg/day. For parenteral administration, although the dosage highly varies with administration routes, a usual dosage is 0.005 to 10 and preferably 0.01 to 1 mg/kg/day. The dosage may be administered in one to several divisions per day.

Generally, pharmaceutical compositions contain the active compound in an effective amount to achieve their intended purpose. In one embodiment, a therapeutically effective amount means an amount effective to prevent or inhibit development or progression of a disease characterized by mycobacterial infection or activity in the subject being treated. Determination of the effective amounts is within the capability of those skilled in the art in light of the description provided herein.

In some embodiments, the medicament of the present invention is suitable for the treatment and/or prevention of diseases and disorders characterized by mycobacterial activity or infection. The mycobacteria may be pathogenic or non-pathogenic. The mycobacteria may be Gram positive or Gram negative.

In some embodiments, the medicament of the present invention is suitable for the treatment in humans (either or both of immunocompetent and immunocompromised) and animals of tuberculous, lepromatous, and non-tuberculous mycobacteria. Non-limiting examples of these include but not limited to the following species and strains: Tuberculous mycobacteria, for example M. tuberculosis, M. bovis, M. africanum, M. microti, M. canetti; Lepromatous mycobacteria, for example M. leprae, M. Lepromatosis; Non-tuberculous mycobacteria, for example M. abscessus, M. abcessus complex, M. avium, M. intracellularae, M. avium complex, M. kansasii, M. malmoense, M. xenopi, M. malmoense, M. flavences, M. scrofulaceum, M, chelonae, M. peregrinum, M. haemophilum, M. fortuitum, M. marinum, M. ulcerans, M. gordonae, M. haemophilum, M. mucogenicum, M. nonchromogenicum, M. terrae, M terrae complex, M. asiaticum, M. celatum, M. shimoidei, M. simiae, M. smegmatis, M. szulgai, M. celatum, M. conspicuum, M. genavense, M. immunogenum, M. xenopi.

In some embodiments, the medicament of the present invention is suitable for the treatment in humans (both immunocompetent and immunocompromised) and animals of non-mycobacterial infectious diseases.

In some embodiments, the subject is known or suspected to need treatment for one or more maladies related to non-pathogenic mycobacterial strain, M. smegmatis, M. vaccae, M. aurum, or combination thereof.

In some embodiments, the subject is known or suspected to need treatment for one or more maladies related to Gram positive bacteria, S. aureus, M luteus, or combination thereof.

In some embodiments, the subject is known or suspected to need treatment for one or more maladies related to Gram negative bacteria, P. aeruginosa, A. baumanii, or combination thereof.

In some embodiments, the subject is known or suspected to need treatment for one or more maladies related to pathogenic mycobacterial strain, M. tuberculosis, M. bovis, M. marinum, M. kansasaii, H37Rv, M. africanum, M. canetti, M. caprae, M. microti, M. mungi, M. pinnipedii, M. leprae, M. avium, Myobacterium tuberculosis complex, tuberculosis, or combination thereof.

In some embodiments, the subject is known or suspected to need treatment for one or more maladies related to non-pathogenic mycobacterial strain, M. smegmatis, M. vaccae, M. aurum, Gram positive bacteria, S. aureus, M. luteus, Gram negative bacteria, P. aeruginosa, A. baumanii, pathogenic mycobacterial strain, M. tuberculosis, M. bovis, M. marinum, M. kansasaii, H37Rv, M. africanum, M. canetti, M. caprae, M. microti, M. mungi, M. pinnipedii, M. avium, Myobacterium tuberculosis complex, tuberculosis, or combination thereof.

In some embodiments, a method is provided, which includes killing or inhibiting the growth of a population of one or more of non-pathogenic mycobacterial strain, M. smegmatis, M. vaccae, M. aurum, Gram positive bacteria, S. aureus, M. luteus, Gram negative bacteria, P. aeruginosa, A. baumanii, pathogenic mycobacterial strain, M. tuberculosis, M. bovis, M. marinum, M. kansasaii, H37Rv, M. africanum, M. canetti, M. caprae, M. microti, M. mungi, M. pinnipedii, M. avium, Myobacterium tuberculosis complex, tuberculosis, or combination thereof, by contacting one or more member of said population with the compounds used in the present invention or composition.

### EXAMPLES

The present invention is explained in more detail below by Examples, but the present invention is not limited to them.

The compound represented by formula (I) of the present invention can be prepared by reference to WO2011/057145, WO2017/049321, WO2011/113606, WO2014/015167, the entire contents of each of which are hereby incorporated by reference, the same as if set forth at length.

Besides, abbreviations used herein have the following meanings:

Me: methyl
Et: ethyl
Bu: butyl
Ph: phenyl
PPh₃: triphenylphosphine
Ac: acetyl
EtOAc: ethyl acetate

### DMF: N, N-dimethylformamide

### DMA: N, N-dimethylacetoamide

TFA: trifluoroacetic acid
DMSO: dimethyl sulfoxide
THF: tetrahydrofuran
WSC: 1-Ethyl-3-(3-dimethylaminopropyl)carbodiimide hydrochloride
HATU: 1-[Bis(dimethylamino)methylene]-1H-1,2,3-triazolo[4,5-b]pyridinium; 3-Oxide, hexafluorophosphate

### DCC: N,N′-Dicyclohhexylcarbodiimide

HOBt: hydrxybenzotriazole
Boc: t-butoxycarbonyl
t: tertially
Cbz: benzyloxycarbonyl
dppf: 1,1′-Bis(diphenylphosphino)ferrocene
Pd₂(dba)₃: Tris(dibezylideneacetone)dipalladium
PdCl₂(dppf): [1,1′-Bis(diphenylphosphino)ferrocene]palladium(II) dichloride
Pd(PPh₃)₄: Tetrakis(triphenylphosphine) palladium(0)
PdCl₂(PPh₃)₂: Bis(triphenylphosphine)palladium chloride

NMR analysis of each example was performed by 400 MHz using deuterated dimethyl sulfoxide (d6-DMSO) or deuterochloroform (CDCl₃). In the case of indicating NMR data, there are cases in which not all measured peaks are described.

“RT” in the specification means a retention time of LC/MS: liquid chromatography/mass spectrometry, and the measurement conditions are as follows.

### (Method A)

UHPLC/MS data of the compounds were measured under the following condition.

### Column: ACQUITY UPLC®BEH C18 (1.7 μm, i.d.50×2.1 mm) (Waters)

Flow rate: 0.8 mL/min
UV detection wavelength: 254 nm
Mobile phase: [A] is 0.1% formic acid-containing aqueous solution, and [B] is 0.1% formic acid-containing acetonitrile solution.
Gradient: Linear gradient of 5% to 100% solvent [B] for 3.5 minutes was performed, and 100% solvent [B] was maintained for 0.5 minute.”

### (Method B)

UHPLC/MS data of the compounds were measured under the following condition.

### Column: ACQUITY UPLC®BEH C18 (1.7 μm, i.d.50×2.1 mm) (Waters)

Flow rate: 0.8 mL/min
UV detection wavelength: 254 nm
Mobile phase: [A] is 10 mM ammonium carbonate-containing aqueous solution, and [B] is acetonitrile.
Gradient: Linear gradient of 5% to 100% solvent [B] for 3.5 minutes was performed, and 100% solvent [B] was maintained for 0.5 minute.

Hereinafter, MS (m/z) indicates the value observed in the mass spectrometry.

### Example 1

### Preparation of Compound I-1-3

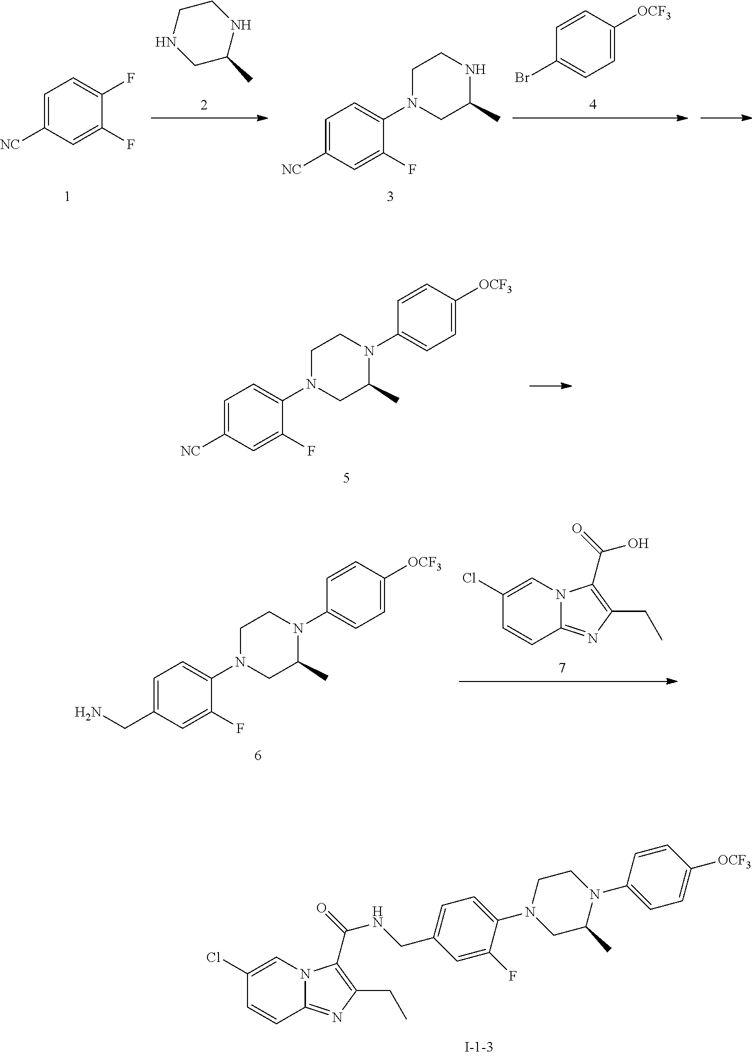

### Step. 1

To a solution of Compound 1 (1 g, 7.19 mmol) in DMA (5 mL) were added K₂CO₃ (1.49 g, 10.78 mmol) and Compound 2 (0.792 g, 7.91 mmol), and the solution was stirred at 100° C. for 4.5 hours. After the reaction solution was poured into water, the mixture was extracted with ethyl acetate. The organic layer was washed with water and brine, then dried with sodium sulfate and filtered. After the organic layer was concentrated under reduced pressure, the residue was washed with hexanes to yield Compound 3 (1.01 g, yield: 64%) as a solid.

1H-NMR (CDCl3) δ: 7.38-7.33 (1H, m), 7.27 (1H, dd, J=12.9, 1.9 Hz), 6.91 (1H, t, J=8.2 Hz), 3.50-3.41 (2H, m), 3.12-2.98 (3H, m), 2.88-2.77 (1H, m), 2.52-2.44 (1H, m), 1.12 (3H, d, J=6.0 Hz).

### Step. 2

To a solution of Compound 3 (510 mg, 2.33 mmol) in toluene (5.0 mL) were added 1-bromo-4-(trifluoromethyl)benzene 4 (576 mg, 2.56 mmol), Pd(OAc)₂ (52.2 mg, 0.233 mmol), 2-dicyclohexylphosphino-2′,6′-diisopropoxy-1,1′-biphenyl (217 mg, 0.465 mmol) and sodium t-butoxide (447 mg, 4.65 mmol), the solution was stirred at 100° C. for 1 hour. To the reaction solution were added water and ethyl acetate. The organic layer was washed with water and brine, dried with anhydrous sodium sulfate and filtered. The organic layer was concentrated under reduced pressure, the residue was purified by silica gel chromatography (hexanes/ethyl acetate) to yield Compound 5 (543 mg, yield: 64%) as a yellow solid.

1H-NMR (CDCl3) δ: 7.39 (1H, dd, J=8.4, 1.9 Hz), 7.30 (1H, dd, J=12.5, 1.9 Hz), 7.14 (2H, d, J=8.3 Hz), 6.95 (1H, t, J=9.2 Hz), 6.92 (2H, d, J=8.3 Hz), 3.99-3.90 (1H, m), 3.55-3.50 (1H, m), 3.42-3.40 (1H, m), 3.35-3.26 (3H, m), 3.20-3.11 (1H, m), 1.16 (3H, d, J=6.4 Hz)

### Step. 3

To a solution of Compound 5 (546 mg, 1.50 mmol) in MeOH (15 mL) were added 5% Pd—C (320 g, 0.150 mmol) and Conc.HCl (1.5 mL), and the solution was stirred at room temperature for 5 hours under hydrogen atmosphere. The reaction mixture was filtered, the filtrate was concentrated under reduced pressure. To the reaction mixture were added aqueous NaOH solution and ethyl acetate. The organic layer was washed with water and brine, dried with anhydrous sodium sulfate and filtered. The organic layer was concentrated under reduced pressure to yield Compound 6 (440 mg, yield: 80%) as a yellow solid.

1H-NMR (CDCl3) δ: 7.16-7.10 (2H, m), 7.07-7.00 (2H, m), 6.98-6.88 (3H, m), 3.99-3.91 (1H, m), 3.86-3.80 (2H, m), 3.40-3.21 (4H, m), 3.17-3.10 (1H, m), 3.05-2.99 (1H, m), 1.19 (3H, d, J=6.4 Hz).

### Step. 4

To a solution of Compound 6 (80 mg, 0.209 mmol) in acetonitrile (2.0 mL) were added Compound 7 (52 mg, 0.230 mmol), triethylamine (0.087 mL, 0.626 mmol), 3-(((ethylimino)methylene)amino)-N,N-dimethylpropan-1-amine (48.6 mg, 0.313 mmol) and HOBt (28.2 mg, 0.209 mmol), the solution was stirred at 80° C. for 1 hour. To the reaction solution was added water, and extracted with ethyl acetate. The organic layer was washed with brine, dried with anhydrous sodium sulfate and filtered. The organic layer was concentrated under reduced pressure, the residue was purified by silica gel chromatography (CHCl₃/methanol) to yield Compound I-1-3 (55 mg, yield: 45%) as a white solid.

LC/MS Method A: m/z=590.2. [M+H]+, retention time: 3.0 min

### Example 2

### Preparation of Compound 8

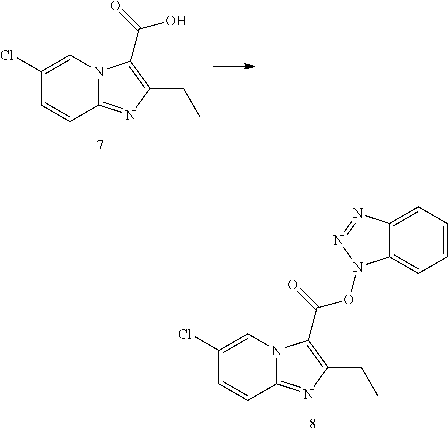 [SMILES: CCc1nc2ccc(Cl)cn2c1C(=O)O ; CCc1nc2ccc(Cl)cn2c1C(=O)On1nnc2ccccc21]

To a solution of Compound 7 (20 g, 89 mmol) in acetonitrile (200 mL) were added N-methylmorpholine (19.6 mL, 178 mmol), 3-(((ethylimino)methylene)amino)-N,N-dimethylpropan-1-amine hydrochloride (18.8 g, 98 mmol) and HOBt (18.01 g, 98 mmol), the solution was stirred at room temperature for over 15 hours. To the reaction solution was added water, and the reaction mixture was filtered. The residue was washed with water and dried under reduced pressure to yield Compound 8 (19.4 g, yield: 64%) as a solid.

1H-NMR (CDCl3) δ: 9.26 (1H, d, J=1.3 Hz), 8.14 (1H, d, J=8.4 Hz), 7.74 (1H, d, J=9.4 Hz), 7.62-7.44 (4H, m), 3.37 (2H, q, J=7.5 Hz), 1.53 (3H, t, J=7.5 Hz).

### Example 3

### Preparation of Compound 10

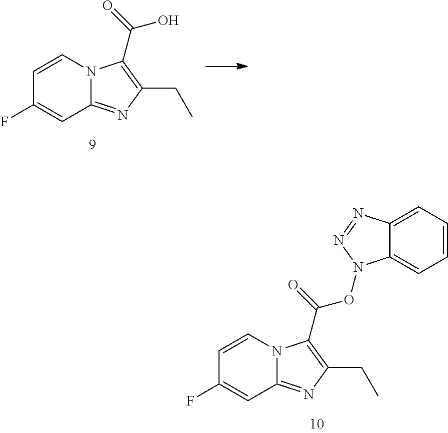 [SMILES: CCc1nc2cc(F)ccn2c1C(=O)O ; CCc1nc2cc(F)ccn2c1C(=O)On1nnc2ccccc21]

To a solution of Compound 9 (4.9 g, 23.7 mmol) in acetonitrile (99 mL) were added N-methylmorpholine (2.86 mL, 26 mmol), 3-(((ethylimino)methylene)amino)-N,N-dimethylpropan-1-amine hydrochloride (4.04 g, 26 mmol) and HOBt (3.2 g, 23.7 mmol), the solution was stirred at room temperature for over 22 hours. To the reaction solution was added water, and the reaction mixture was filtered. The residue was washed with water and dried under reduced pressure to yield Compound 10 (5.09 g, yield: 66%) as a white solid.

1H-NMR (CDCl3) δ: 9.18 (1H, dd, J=7.5, 5.4 Hz), 8.14 (1H, dt, J=8.4, 0.9 Hz), 7.59 (1H, dd, J=16.1, 8.0 Hz), 7.54-7.40 (3H, m), 6.98 (1H, td, J=7.5, 2.7 Hz), 3.35 (2H, q, J=7.5 Hz), 1.52 (3H, t, J=7.5 Hz).

### Example 4

### Preparation of Compound I-1-38

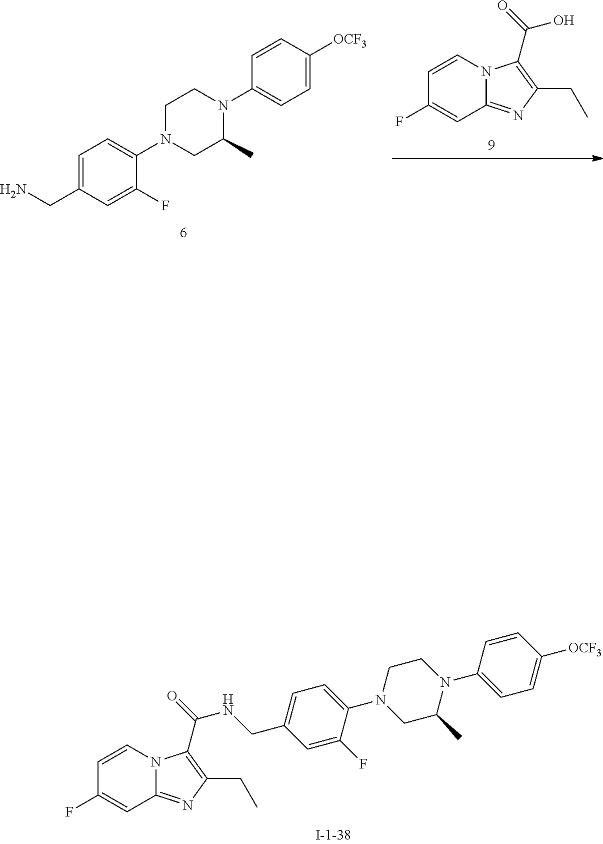 [SMILES: C[C@H]1CN(c2ccc(CN)cc2F)CCN1c1ccc(OC(F)(F)F)cc1 ; CCc1nc2cc(F)ccn2c1C(=O)O ; CCc1nc2cc(F)ccn2c1C(=O)NCc1ccc(N2CCN(c3ccc(OC(F)(F)F)cc3)[C@@H](C)C2)c(F)c1]

To a solution of Compound 6 (70 mg, 0.183 mmol) in acetonitrile (1.0 mL) were added Compound 9 (41.8 mg, 0.201 mmol), triethylamine (0.051 mL, 0.365 mmol), 3-(((ethylimino)methylene)amino)-N,N-dimethylpropan-1-amine hydrochloride (52.5 mg, 0.274 mmol) and HOBt (24.7 mg, 0.183 mmol), the solution was stirred at 50° C. for 1 hour. To the reaction solution was added saturated aqueous NaHCO₃ solution, and extracted with ethyl acetate. The organic layer was washed with water and brine, and dried with anhydrous magnesium sulfate. The organic layer was filtered and concentrated under reduced pressure, the residue was purified by silica gel chromatography (hexane/ethyl acetate) to yield Compound I-1-38 (78 mg, yield: 75%) as a white solid.

1H-NMR (CDCl3) δ: 9.44 (1H, t, J=5.6 Hz), 7.22 (1H, s), 7.14-7.07 (4H, m), 6.97-6.92 (3H, m), 6.80 (1H, t, J=7.3 Hz), 6.06 (1H, s), 4.63 (2H, d, J=3.8 Hz), 3.95 (1H, d, J=2.0 Hz), 3.38-3.27 (4H, m), 3.17-3.14 (1H, m), 3.06-2.95 (3H, m), 1.44-1.40 (3H, m), 1.19 (3H, d, J=4.5 Hz).

LC/MS Method B: m/z=574.25 [M+H]+, retention time: 2.99 min

### Example 5

### Preparation of Compound I-1-2

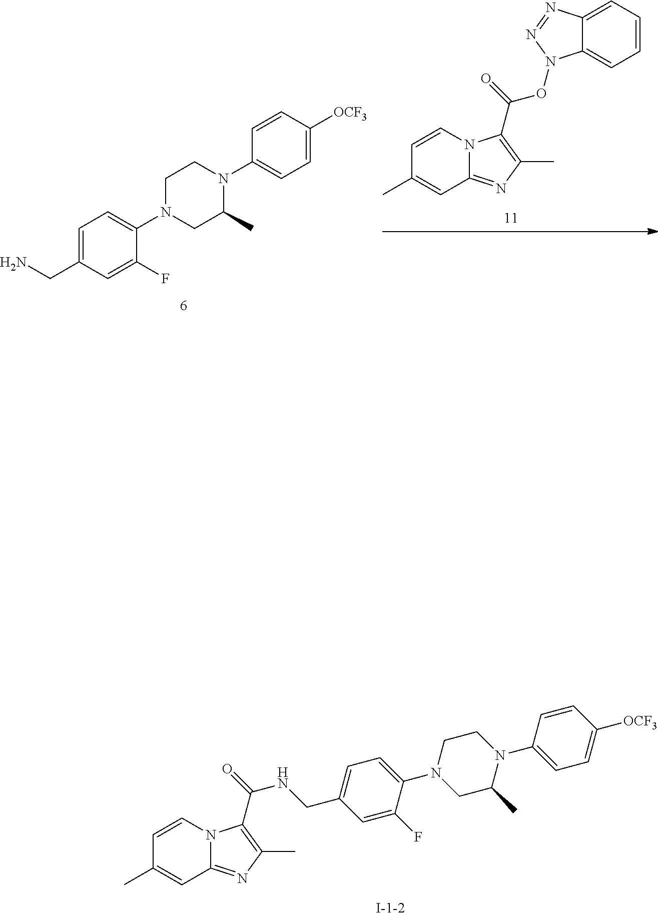 [SMILES: C[C@H]1CN(c2ccc(CN)cc2F)CCN1c1ccc(OC(F)(F)F)cc1 ; Cc1ccn2c(C(=O)On3nnc4ccccc43)c(C)nc2c1 ; Cc1ccn2c(C(=O)NCc3ccc(N4CCN(c5ccc(OC(F)(F)F)cc5)[C@@H](C)C4)c(F)c3)c(C)nc2c1]

To a solution of Compound 6 (1.5 g, 3.91 mmol) in dichloromethane (1.0 mL) were added Compound 11 (1.32 g, 4.3 mmol) and triethylamine (0.814 mL, 5.87 mmol), the solution was stirred at room temperature for 15 hours. The reaction solution was purified by aminosilica gel and silica gel chromatography (CHCl₃/methanol) to yield Compound I-1-2 (1.76 g, yield: 81%) as a white solid.

LC/MS Method A: m/z=574.25 [M+H]+, retention time: 2.99 min

### Example 6

### Preparation of Compound I-1-70

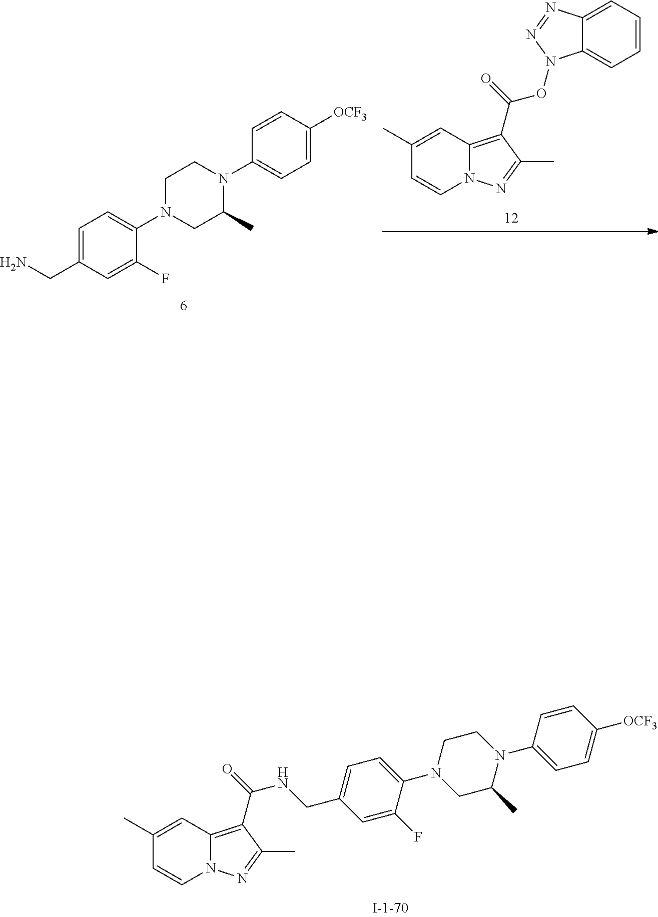

To a solution of Compound 6 (80 mg, 0.209 mmol) in dichloromethane (0.8 mL) were added Compound 12 (64 g, 0.209 mmol) and triethylamine (0.087 mL, 0.626 mmol), the solution was stirred at room temperature for 15 hours. The reaction solution was purified by aminosilica gel and silica gel chromatography (CHCl₃/methanol) to yield Compound I-1-70 (47 mg, yield: 41%) as a white solid.

LC/MS Method A: m/z=556.25 [M+H]+, retention time: 2.94 min

### Example 7

### Preparation of Compound I-1-25

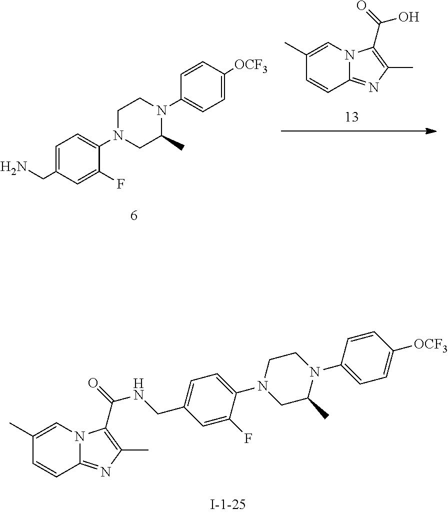

To a solution of Compound 6 (50 mg, 0.130 mmol) in dichloromethane (0.8 mL) were added Compound 13 (29.8 g, 0.156 mmol), triethylamine (0.054 mL, 0.391 mmol), 3-(((ethylimino)methylene)amino)-N,N-dimethylpropan-1-amine (30.4 mg, 0.196 mmol) and HOBt (8.8 mg, 0.065 mmol), the solution was stirred at room temperature for 15 hours. To the reaction solution was added saturated aqueous NaHCO₃ solution, and extracted with dichloromethane. The organic layer was washed with water and brine, and dried with anhydrous magnesium sulfate. The organic layer was filtered and concentrated under reduced pressure. The residue was purified by silica gel chromatography (CHCl₃/methanol) to yield Compound I-1-25 (47 mg, yield: 74.5%) as a white solid.

LC/MS Method A: m/z=556.3 [M+H]+, retention time: 2.73 min

### Example 81

### Preparation of Compound I-1-1

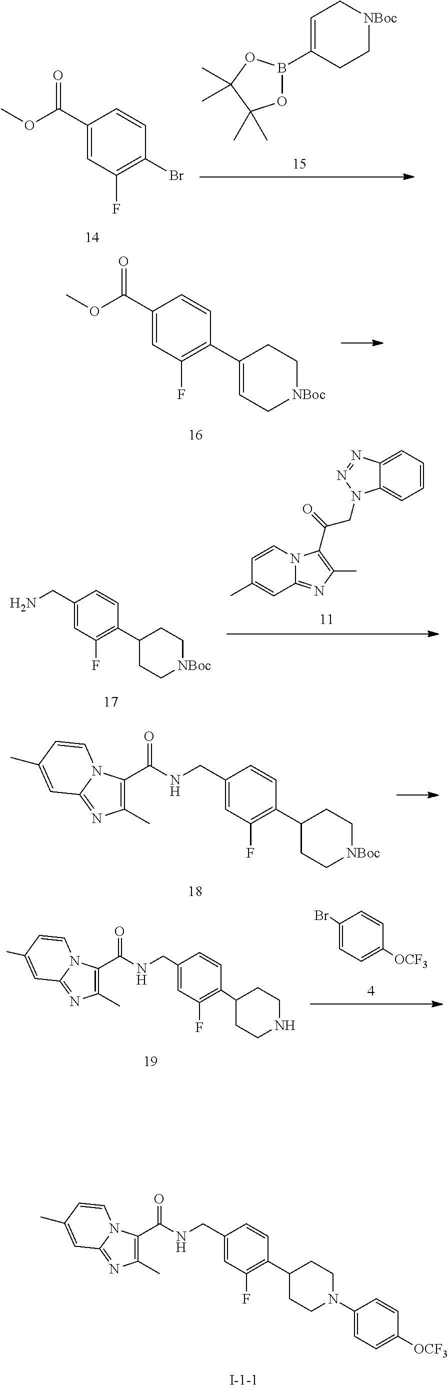

### Step. 1

To a solution of Compound 14 (5.05 g, 21.7 mmol) in 1,4-dioxane (76 mL) were added compound 15 (7.37 g, 23.8 mmol), PdCl₂(PPh₃)₂ (1.52 g, 2.17 mmol) and 2M. Na₂CO₃ aqueous solution (32.5 ml, 65 mmol). The reaction mixture was stirred at 100° C. for 1.5 hours. After the reaction solution was poured into water, the mixture was extracted with ethyl acetate. The organic layer was washed with water and brine, dried over anhydrous sodium sulfate, filtered, and concentrated under reduced pressure. The residue was purified by silica gel column chromatography (hexanes-EtOAc) to yield compound 16 (6.59 g, 19.7 mmol, 91%) as a white solid.

1H-NMR (400 MHz, CDCl3) δ: 7.78 (dd, 1H, J=7.8, 1.3 Hz), 7.70 (dd, 1H, 11.6, 1.3 Hz), 6.04 (m, 1H), 4.15-4.05 (m, 2H), 3.92 (s, 3H), 3.62 (t, 2H, 5.4), 2.56-2.46 (m, 2H), 1.48 (s, 9H)

### Step 2

To a solution of compound 16 (6.49 g, 19.4 mmol) in THF (100 ml), purged with inert atmosphere, was added 10% Pd(OH)₂/C (5.44 g, 1.94 mmol). The reaction mixture was charged with hydrogen gas (1 atm). The resulting suspension was vigorously stirred for 6.5 hours. The reaction mixture was purged with an inert atmosphere, filtered through a celite pad. The filter cake was washed with EtOAc, and the filtrate was concentrated under reduce pressure to yield the crude material as an oil. This crude material was used for the next reaction without further purification. The crude material was dissolved in THF (100 ml) and was added to a suspension of LiAlH₄ (1.46 g, 38.5 mmol) in THF (100 ml) at 0° C. After stirring for 5 min, to the reaction mixture was added sodium sulfate decahydrate (24.83 g, 77 mmol) and the reaction was stirred for 3 hours. The reaction mixture was filtered through a celite pad. The filter cake was washed with EtOAc, and the filtrate was concentrated under reduce pressure to yield the crude product as an oil. This crude material was used in the next reaction without further purification. To a solution of the crude material in dichloromethane (100 ml) were added Et₃N (7.93 ml, 57.2 mmol) and methansulfonyl chloride (3.28 g, 28.6 mmol) at −78° C. To the reaction solution was added H₂O, and the mixture was extracted with ethyl acetate twice. The organic layer was washed with water and brine, dried over anhydrous sodium sulfate, filtered, and concentrated under reduced pressure to yield the crude material as an oil. This crude material was used in the next reaction without further purification. To a solution of the crude material in DMF (60 ml) was added NaN₃ (2.47 g, 37.9 mmol) and the mixture was stirred at 80° C. for 1 hour. To the reaction solution was added H₂O, and the mixture was extracted with ethyl acetate. The organic layer was washed with water and brine, dried over anhydrous sodium sulfate, filtered, and concentrated under reduced pressure to yield the crude material as an oil. This crude product was used to the next reaction without further purification. To a solution of the crude product in THF (50 ml) were added PPh₃ (5.55 g, 21.2 mmol) and H₂O (3.18 ml, 176 mmol) and the mixture was stirred at reflux for 2 hours. The mixture was then concentrated under reduced pressure, the residue was purified by silica gel column chromatography (EtOAc-CHCl₃-MeOH) to yield compound 17 (4.11 g, 13.3 mmol, 69% for 5 steps) as a white solid.

1H-NMR (400 MHz, CDCl3) δ: 7.15 (t, 1H, J=7.7 Hz), 7.04 (d, 1H, J=7.9 Hz), 7.00 (d, 1H, J=11.3 Hz), 4.24 (s, 2H), 3.84 (s, 2H), 2.98-2.89 (m, 1H), 2.84-2.70 (m, 2H), 1.83-1.75 (m, 2H), 1.68-1.55 (4H, m), 1.48 (s, 9H)

LC/MS Method: A, LC-MS: m/z=253. [M+H]+, retention time: 1.67 min

### Step 3

To a solution of compound 17 (1.84 g, 5.97 mmol) in dichloromethane (20 ml) were added compound 11 (2.02 g, 6.56 mmol) and Et₃N (1.24 ml, 8.95 mmol) and the reaction was stirred at room temperature for 1 hour. The reaction mixture was then concentrated under reduced pressure and the residue was purified by silica gel chromatography (CHCl₃/MeOH) to yield compound 18 (2.83 g, 5.89 mmol, 99%) as a white solid.

LC/MS Method: A, LC-MS: m/z=481. [M+H]+, retention time: 2.17 min

### Step 4

To a solution of compound 18 (2.44 g, 5.08 mmol) in dichloromethane (20 ml) was added TFA (3.91 ml, 50.8 mmol) and the reaction was stirred at room temperature for 1 hour. The reaction mixture was then concentrated under reduced pressure and the residue was purified by amino silica gel chromatography (CHCl₃/MeOH) to yield compound 19 (1.83 g, 4.81 mmol, 95%) as a off-white amorphous.

LC/MS Method: A, LC-MS: m/z=381. [M+H]+, retention time: 1.00 min

### Step 5

To a solution of compound 19 (100 mg, 0.263 mmol) in THF were added compound 4 (95 mg, 0.394 mmol), Pd₂(dba)₃ (24.1 mg, 0.0026 mmol), 2-dicyclohexylphosphino-2′,6′-diisopropoxy-1,1′-biphenyl (49.1 mg, 0.105 mmol) and NaOtBu (0.789 mmol), and then the reaction was stirred at 60° C. for 1 hour. To the reaction solution was added H₂O, and the mixture was extracted with ethyl acetate. The organic layer was washed with water and brine, dried over anhydrous sodium sulfate, filtered, and concentrated under reduced pressure. The residue was purified by silica gel chromatography (hexanes/EtOAc) to yield compound I-1-1 (81.7 mg, 0.151 mmol, 57.5%)

1H-NMR (400 MHz, CDCl3) δ: 9.32 (d, 1H, J=7.5 Hz), 7.33 (s, 1H), 7.28-7.22 (m, 1H), 7.15-7.04 (m, 4H), 6.94 (d, 2H, J=7.9 Hz), 6.77 (d, 1H, J=6.5 Hz), 6.06 (s, 1H), 4.67 (d, 2H, J=5.3 Hz), 3.80-3.72 (m, 2H), 3.05-2.95 (m, 1H), 2.90-2.80 (m, 2H), 2.69 (s, 3H), 2.43 (s, 3H), 1.95-1.83 (m, 4H)

LC/MS Method: A, LC-MS: m/z=541. [M+H]+, retention time: 2.42 min

The following Compounds were obtained in accordance with the general synthetic methods and Examples. The chemical structures and the physical properties (LC/MS data) of Compounds are described below.

TABLE 1

| | | LCMS | | MS |
|---|---|---|---|---|
| No. | Structure | method | RT | (M + H) |
| I-1-1 | 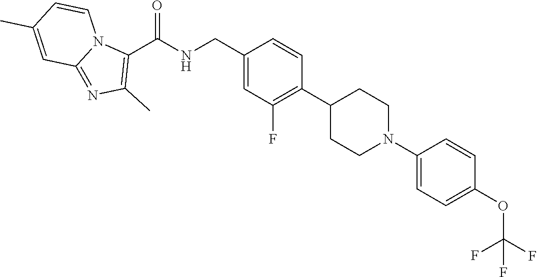 [SMILES: Cc1ccn2c(C(=O)NCc3ccc(C4CCN(c5ccc(OC(F)(F)F)cc5)CC4)c(F)c3)c(C)nc2c1] | A | 2.42 | 541.2 |
| I-1-2 | 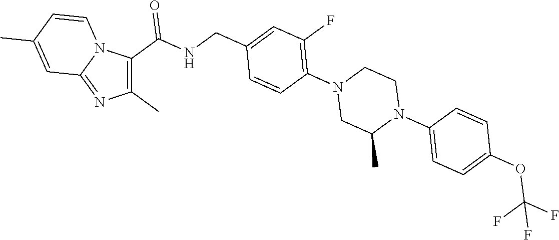 [SMILES: Cc1ccn2c(C(=O)NCc3ccc(N4CCN(c5ccc(OC(F)(F)F)cc5)[C@@H](C)C4)c(F)c3)c(C)nc2c1] | A | 2.52 | 556.3 |
| I-1-3 | 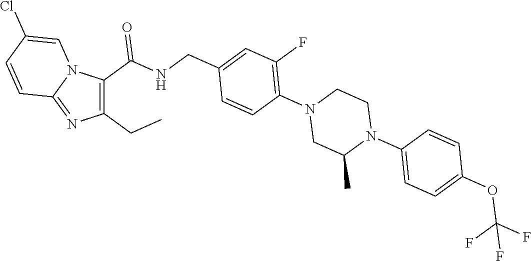 [SMILES: CCc1nc2ccc(Cl)cn2c1C(=O)NCc1ccc(N2CCN(c3ccc(OC(F)(F)F)cc3)[C@@H](C)C2)c(F)c1] | A | 3 | 590.2 |
| I-1-4 | 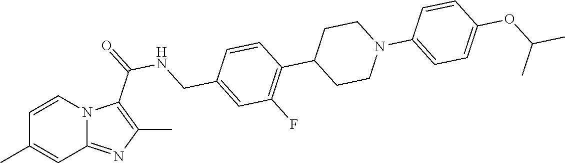 [SMILES: Cc1ccn2c(C(=O)NCc3ccc(C4CCN(c5ccc(OC(C)C)cc5)CC4)c(F)c3)c(C)nc2c1] | A | 1.8 | 515.3 |
| I-1-5 | 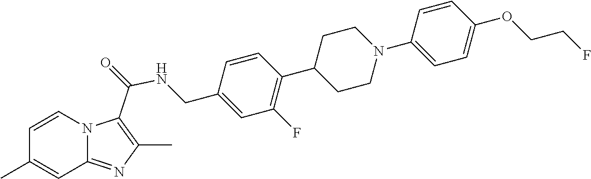 [SMILES: Cc1ccn2c(C(=O)NCc3ccc(C4CCN(c5ccc(OCCF)cc5)CC4)c(F)c3)c(C)nc2c1] | A | 1.57 | 519.2 |
| I-1-6 | 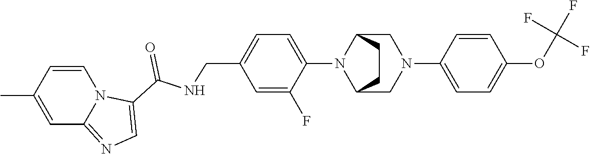 [SMILES: Cc1ccn2c(C(=O)NCc3ccc(N4[C@@H]5CC[C@H]4CN(c4ccc(OC(F)(F)F)cc4)C5)c(F)c3)cnc2c1] | A | 2.6 | 568.2 |

TABLE 2

| | | | | |
|---|---|---|---|---|
| I-1-7 | 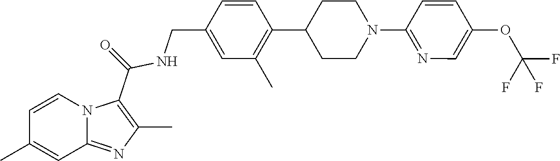 [SMILES: Cc1ccn2c(C(=O)NCc3ccc(C4CCN(c5ccc(OC(F)(F)F)cn5)CC4)c(C)c3)c(C)nc2c1] | A | 2.45 | 541.21 |
| I-1-8 | 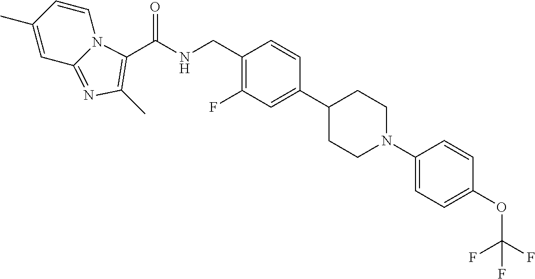 [SMILES: Cc1ccn2c(C(=O)NCc3ccc(C4CCN(c5ccc(OC(F)(F)F)cc5)CC4)cc3F)c(C)nc2c1] | A | 2.57 | 541.2 |
| I-1-9 | 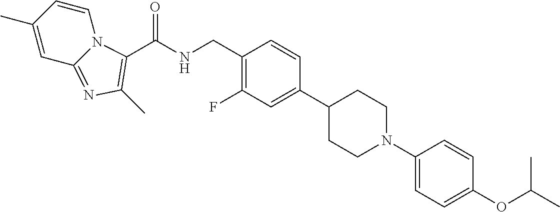 [SMILES: Cc1ccn2c(C(=O)NCc3ccc(C4CCN(c5ccc(OC(C)C)cc5)CC4)cc3F)c(C)nc2c1] | A | 1.75 | 515.25 |
| I-1-10 | 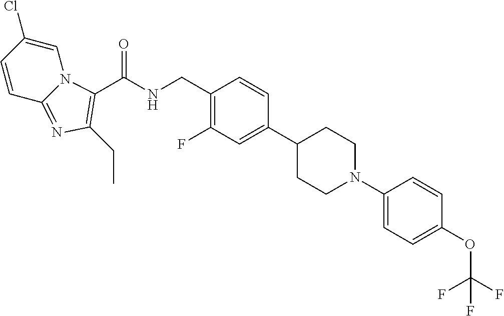 [SMILES: CCc1nc2ccc(Cl)cn2c1C(=O)NCc1ccc(C2CCN(c3ccc(OC(F)(F)F)cc3)CC2)cc1F] | A | 2.98 | 575.2 |
| I-1-11 | 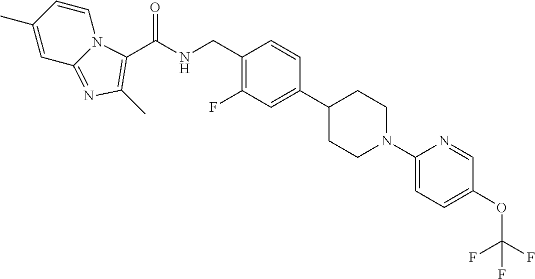 [SMILES: Cc1ccn2c(C(=O)NCc3ccc(C4CCN(c5ccc(OC(F)(F)F)cn5)CC4)cc3F)c(C)nc2c1] | A | 2.47 | 542.2 |
| I-1-12 | 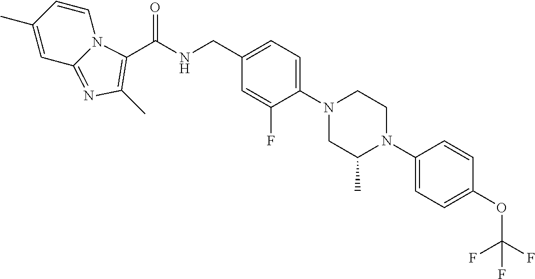 [SMILES: Cc1ccn2c(C(=O)NCc3ccc(N4CCN(c5ccc(OC(F)(F)F)cc5)[C@H](C)C4)c(F)c3)c(C)nc2c1] | A | 2.56 | 556.2 |

TABLE 3

| | | | | |
|---|---|---|---|---|
| I-1-13 | 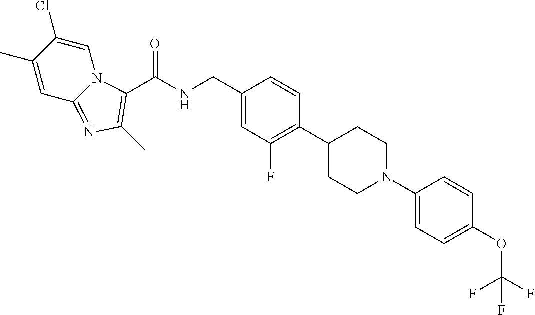 [SMILES: Cc1cc2nc(C)c(C(=O)NCc3ccc(C4CCN(c5ccc(OC(F)(F)F)cc5)CC4)c(F)c3)n2cc1Cl] | A | 3.02 | 575.2 |
| I-1-14 | 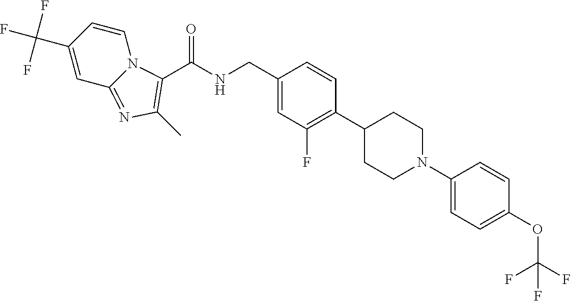 [SMILES: Cc1nc2cc(C(F)(F)F)ccn2c1C(=O)NCc1ccc(C2CCN(c3ccc(OC(F)(F)F)cc3)CC2)c(F)c1] | A | 3.11 | 595.2 |
| I-1-15 | 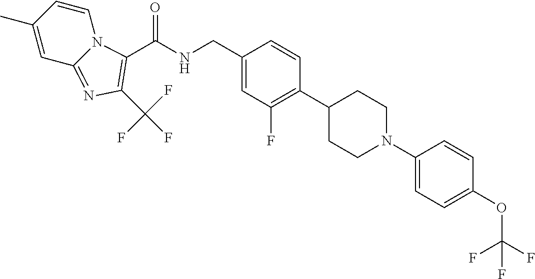 [SMILES: Cc1ccn2c(C(=O)NCc3ccc(C4CCN(c5ccc(OC(F)(F)F)cc5)CC4)c(F)c3)c(C(F)(F)F)nc2c1] | A | 3.07 | 595.2 |
| I-1-16 | 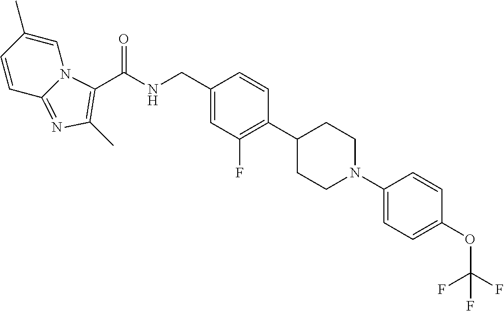 [SMILES: Cc1ccc2nc(C)c(C(=O)NCc3ccc(C4CCN(c5ccc(OC(F)(F)F)cc5)CC4)c(F)c3)n2c1] | A | 2.62 | 541.3 |
| I-1-17 | 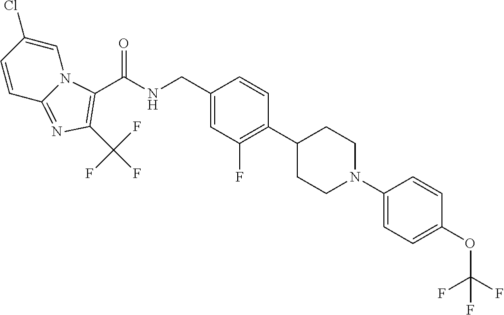 [SMILES: O=C(NCc1ccc(C2CCN(c3ccc(OC(F)(F)F)cc3)CC2)c(F)c1)c1c(C(F)(F)F)nc2ccc(Cl)cn12] | A | 3.21 | 615.2 |
| I-1-18 | 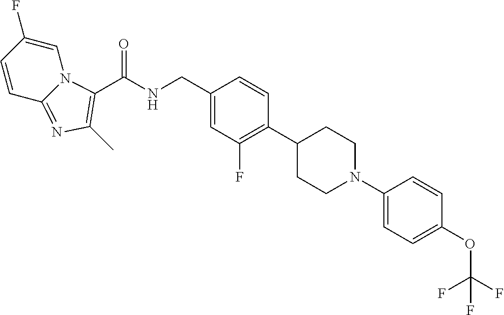 [SMILES: Cc1nc2ccc(F)cn2c1C(=O)NCc1ccc(C2CCN(c3ccc(OC(F)(F)F)cc3)CC2)c(F)c1] | A | 2.9 | 545.2 |

TABLE 4

| | | | | |
|---|---|---|---|---|
| I-1-19 | 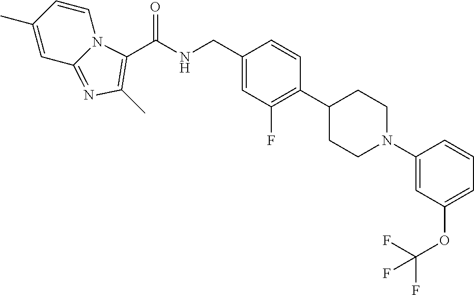 [SMILES: Cc1ccn2c(C(=O)NCc3ccc(C4CCN(c5cccc(OC(F)(F)F)c5)CC4)c(F)c3)c(C)nc2c1] | A | 2.68 | 541.2 |
| I-1-20 | 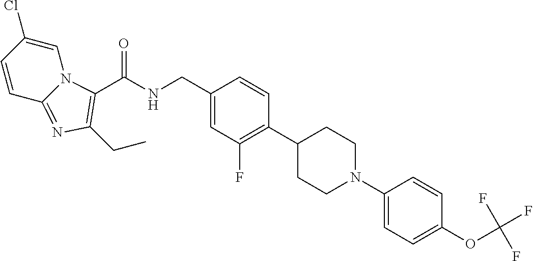 [SMILES: CCc1nc2ccc(Cl)cn2c1C(=O)NCc1ccc(C2CCN(c3ccc(OC(F)(F)F)cc3)CC2)c(F)c1] | A | 3.12 | 575.2 |
| I-1-21 | 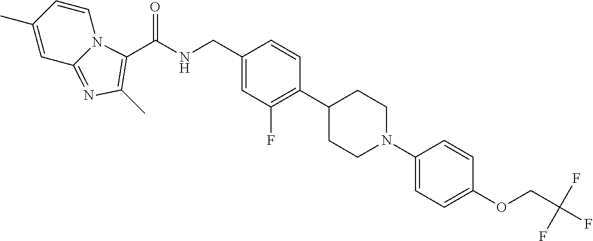 [SMILES: Cc1ccn2c(C(=O)NCc3ccc(C4CCN(c5ccc(OCC(F)(F)F)cc5)CC4)c(F)c3)c(C)nc2c1] | A | 1.88 | 555.3 |
| I-1-22 | 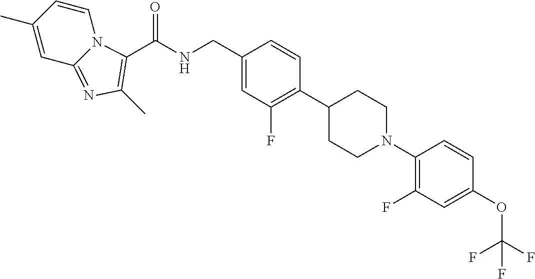 [SMILES: Cc1ccn2c(C(=O)NCc3ccc(C4CCN(c5ccc(OC(F)(F)F)cc5F)CC4)c(F)c3)c(C)nc2c1] | A | 2.76 | 559.3 |
| I-1-23 | 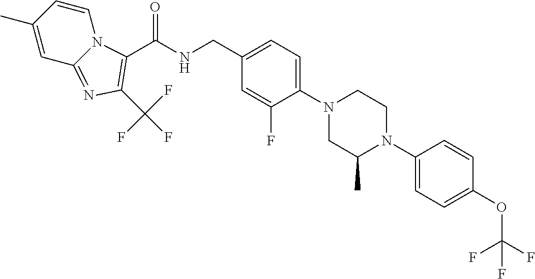 [SMILES: Cc1ccn2c(C(=O)NCc3ccc(N4CCN(c5ccc(OC(F)(F)F)cc5)[C@@H](C)C4)c(F)c3)c(C(F)(F)F)nc2c1] | A | 3.09 | 610.2 |
| I-1-24 | 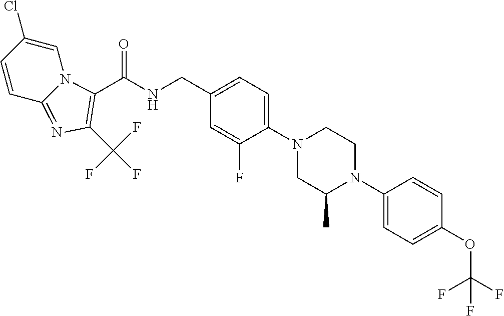 [SMILES: C[C@H]1CN(c2ccc(CNC(=O)c3c(C(F)(F)F)nc4ccc(Cl)cn34)cc2F)CCN1c1ccc(OC(F)(F)F)cc1] | A | 3.2 | 630.2 |

TABLE 5

| | | | | |
|---|---|---|---|---|
| I-1-25 | 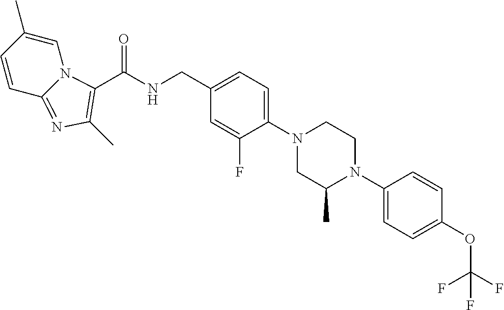 [SMILES: Cc1ccc2nc(C)c(C(=O)NCc3ccc(N4CCN(c5ccc(OC(F)(F)F)cc5)[C@@H](C)C4)c(F)c3)n2c1] | A | 2.73 | 556.3 |
| I-1-26 | 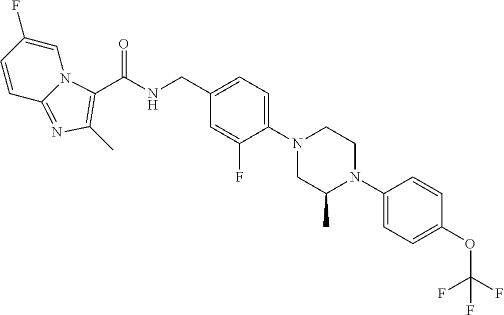 [SMILES: Cc1nc2ccc(F)cn2c1C(=O)NCc1ccc(N2CCN(c3ccc(OC(F)(F)F)cc3)[C@@H](C)C2)c(F)c1] | A | 2.78 | 560.2 |
| I-1-27 | 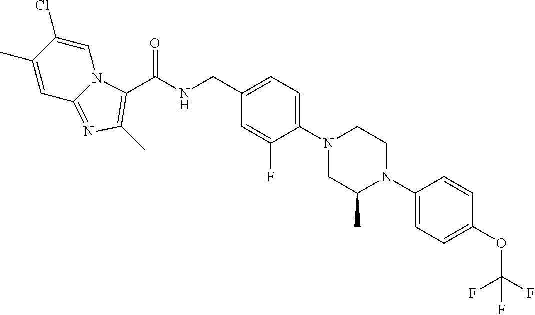 [SMILES: Cc1cc2nc(C)c(C(=O)NCc3ccc(N4CCN(c5ccc(OC(F)(F)F)cc5)[C@@H](C)C4)c(F)c3)n2cc1Cl] | A | 3.09 | 590.2 |
| I-1-28 | 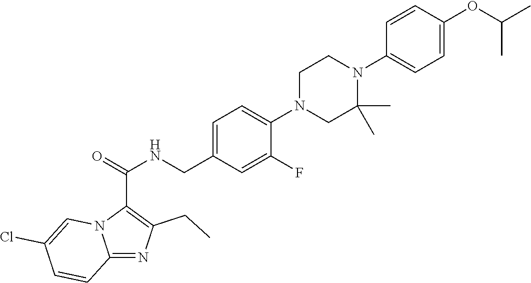 [SMILES: CCc1nc2ccc(Cl)cn2c1C(=O)NCc1ccc(N2CCN(c3ccc(OC(C)C)cc3)C(C)(C)C2)c(F)c1] | B | 3.26 | 578.3 |
| I-1-29 | 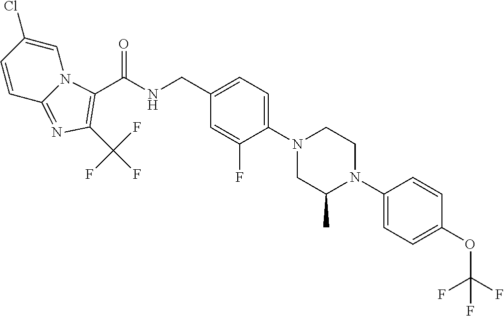 [SMILES: C[C@H]1CN(c2ccc(CNC(=O)c3c(C(F)(F)F)nc4ccc(Cl)cn34)cc2F)CCN1c1ccc(OC(F)(F)F)cc1] | A | 3.19 | 576.2 |
| I-1-30 | 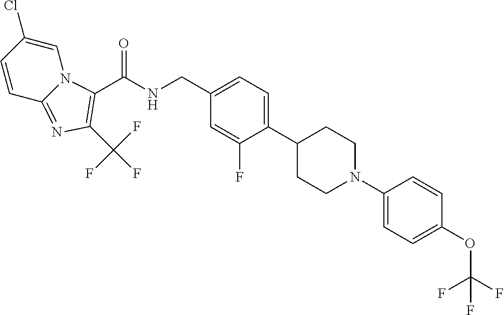 [SMILES: O=C(NCc1ccc(C2CCN(c3ccc(OC(F)(F)F)cc3)CC2)c(F)c1)c1c(C(F)(F)F)nc2ccc(Cl)cn12] | A | 3.14 | 560.25 |

TABLE 6

| | | | | |
|---|---|---|---|---|
| I-1-31 | 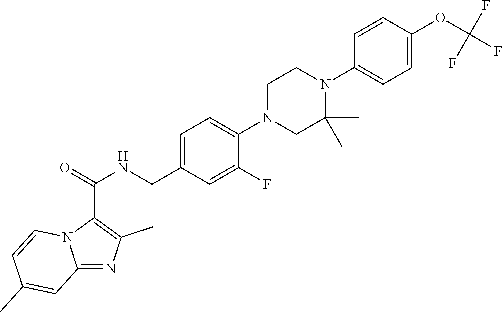 [SMILES: Cc1ccn2c(C(=O)NCc3ccc(N4CCN(c5ccc(OC(F)(F)F)cc5)C(C)(C)C4)c(F)c3)c(C)nc2c1] | B | 2.95 | 570.25 |
| I-1-32 | 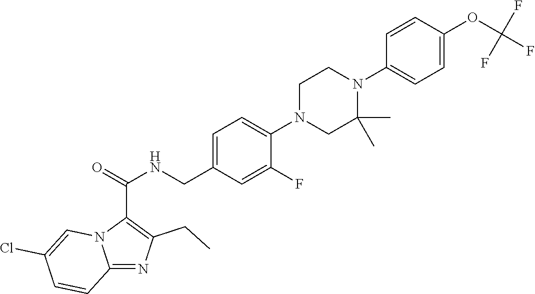 [SMILES: CCc1nc2ccc(Cl)cn2c1C(=O)NCc1ccc(N2CCN(c3ccc(OC(F)(F)F)cc3)C(C)(C)C2)c(F)c1] | B | 3.3 | 604.2 |
| I-1-33 | 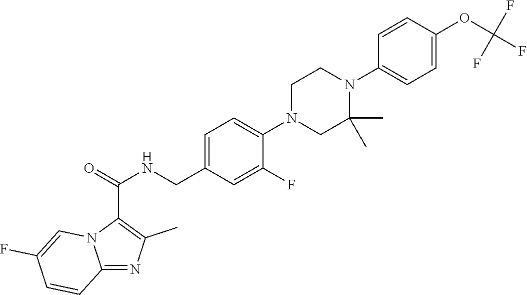 [SMILES: Cc1nc2ccc(F)cn2c1C(=O)NCc1ccc(N2CCN(c3ccc(OC(F)(F)F)cc3)C(C)(C)C2)c(F)c1] | B | 3.06 | 574.25 |
| I-1-34 | 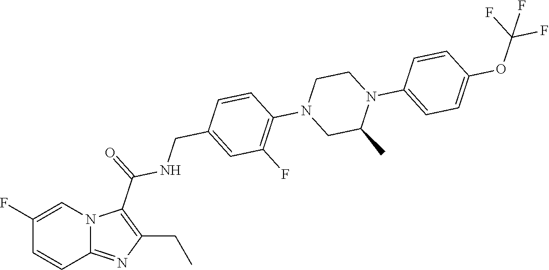 [SMILES: CCc1nc2ccc(F)cn2c1C(=O)NCc1ccc(N2CCN(c3ccc(OC(F)(F)F)cc3)[C@@H](C)C2)c(F)c1] | A | 2.77 | 574.28 |
| I-1-35 | 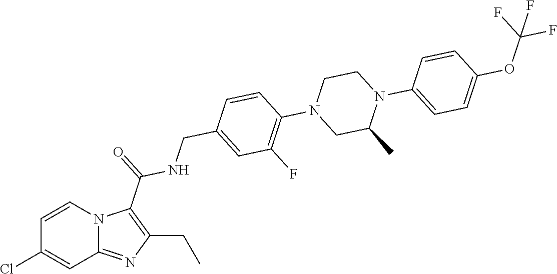 [SMILES: CCc1nc2cc(Cl)ccn2c1C(=O)NCc1ccc(N2CCN(c3ccc(OC(F)(F)F)cc3)[C@@H](C)C2)c(F)c1] | A | 2.91 | 590.2 |
| I-1-36 | 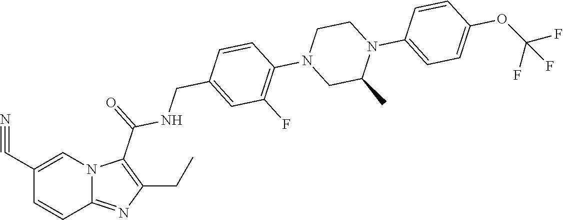 [SMILES: CCc1nc2ccc(C#N)cn2c1C(=O)NCc1ccc(N2CCN(c3ccc(OC(F)(F)F)cc3)[C@@H](C)C2)c(F)c1] | A | 2.95 | 581.25 |

TABLE 7

| | | | | |
|---|---|---|---|---|
| I-1-37 | 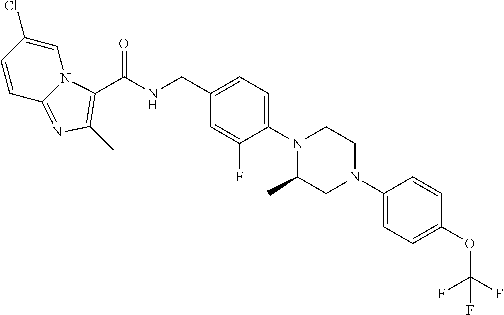 [SMILES: Cc1nc2ccc(Cl)cn2c1C(=O)NCc1ccc(N2CCN(c3ccc(OC(F)(F)F)cc3)C[C@H]2C)c(F)c1] | A | 3.24 | 576.2 |
| I-1-38 | 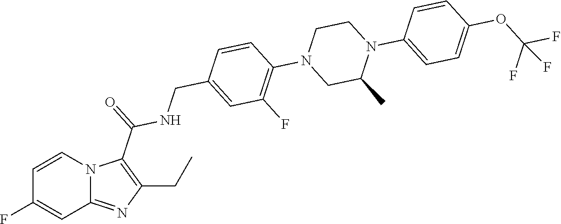 [SMILES: CCc1nc2cc(F)ccn2c1C(=O)NCc1ccc(N2CCN(c3ccc(OC(F)(F)F)cc3)[C@@H](C)C2)c(F)c1] | B | 2.99 | 574.25 |
| I-1-39 | 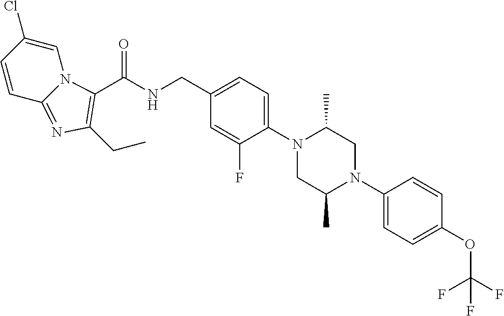 [SMILES: CCc1nc2ccc(Cl)cn2c1C(=O)NCc1ccc(N2C[C@H](C)N(c3ccc(OC(F)(F)F)cc3)C[C@H]2C)c(F)c1] | A | 3.65 | 604.2 |
| I-1-40 | 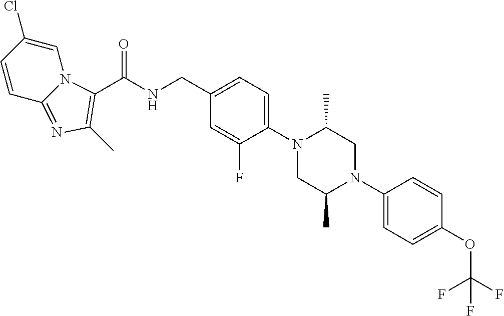 [SMILES: Cc1nc2ccc(Cl)cn2c1C(=O)NCc1ccc(N2C[C@H](C)N(c3ccc(OC(F)(F)F)cc3)C[C@H]2C)c(F)c1] | A | 3.36 | 590.2 |
| I-1-41 | 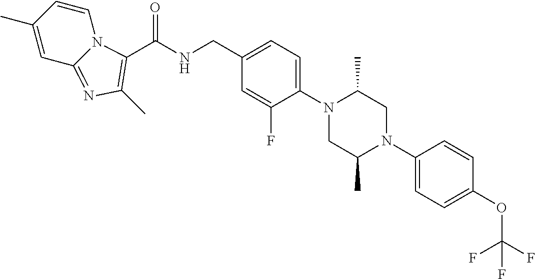 [SMILES: Cc1ccn2c(C(=O)NCc3ccc(N4C[C@H](C)N(c5ccc(OC(F)(F)F)cc5)C[C@H]4C)c(F)c3)c(C)nc2c1] | A | 3.32 | 570.2 |
| I-1-42 | 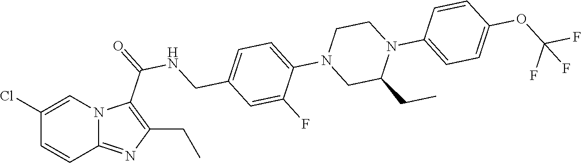 [SMILES: CCc1nc2ccc(Cl)cn2c1C(=O)NCc1ccc(N2CCN(c3ccc(OC(F)(F)F)cc3)[C@@H](CC)C2)c(F)c1] | A | 3.09 | 604.2 |

TABLE 8

| | | | | |
|---|---|---|---|---|
| I-1-43 | 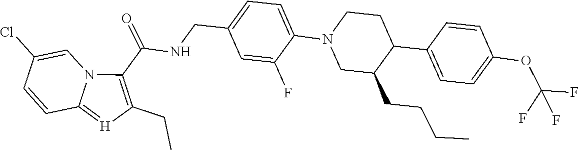 | A | 3.36 | 632.2 |
| I-1-44 | 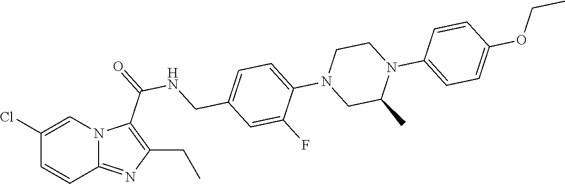 [SMILES: CCOc1ccc(N2CCN(c3ccc(CNC(=O)c4c(CC)nc5ccc(Cl)cn45)cc3F)C[C@@H]2C)cc1] | A | 1.75 | 550.2 |
| I-1-45 | 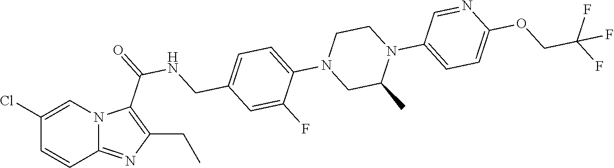 [SMILES: CCc1nc2ccc(Cl)cn2c1C(=O)NCc1ccc(N2CCN(c3ccc(OCC(F)(F)F)nc3)[C@@H](C)C2)c(F)c1] | A | 2.663 | 605.2 |
| I-1-46 | 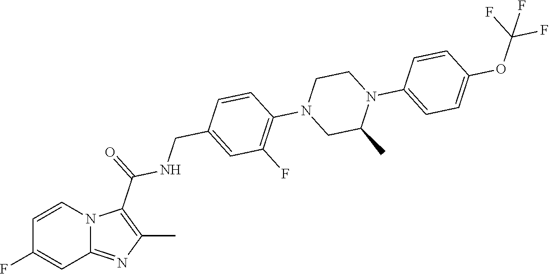 [SMILES: Cc1nc2cc(F)ccn2c1C(=O)NCc1ccc(N2CCN(c3ccc(OC(F)(F)F)cc3)[C@@H](C)C2)c(F)c1] | A | 2.42 | 560.25 |
| I-1-47 | 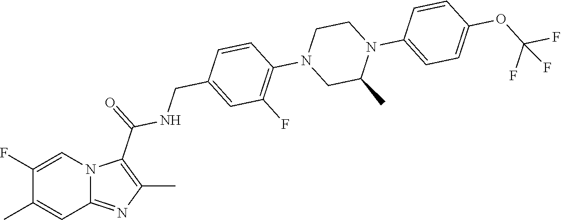 [SMILES: Cc1cc2nc(C)c(C(=O)NCc3ccc(N4CCN(c5ccc(OC(F)(F)F)cc5)[C@@H](C)C4)c(F)c3)n2cc1F] | A | 2.41 | 574.25 |
| I-1-48 | 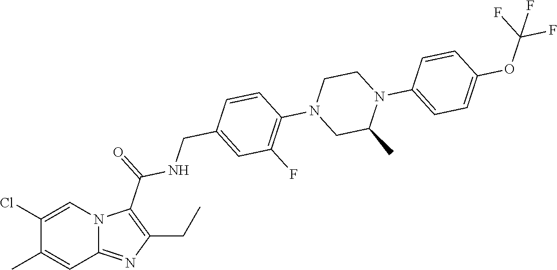 [SMILES: CCc1nc2cc(C)c(Cl)cn2c1C(=O)NCc1ccc(N2CCN(c3ccc(OC(F)(F)F)cc3)[C@@H](C)C2)c(F)c1] | A | 2.74 | 604.2 |

TABLE 9

| | | | | |
|---|---|---|---|---|
| I-1-49 | 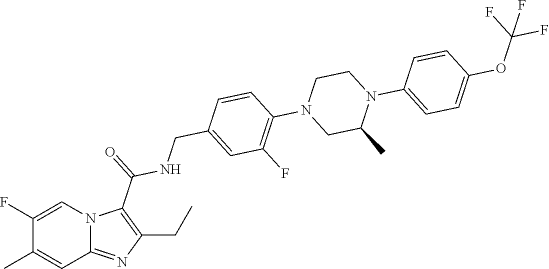 [SMILES: CCc1nc2cc(C)c(F)cn2c1C(=O)NCc1ccc(N2CCN(c3ccc(OC(F)(F)F)cc3)[C@@H](C)C2)c(F)c1] | A | 2.5 | 588.25 |
| I-1-50 | 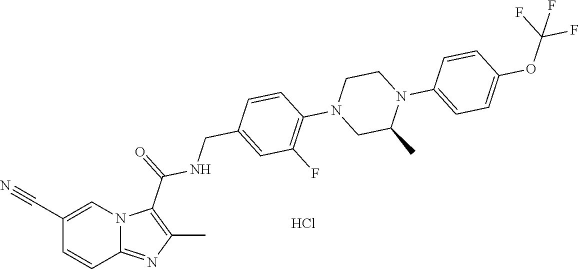 [SMILES: Cc1nc2ccc(C#N)cn2c1C(=O)NCc1ccc(N2CCN(c3ccc(OC(F)(F)F)cc3)[C@@H](C)C2)c(F)c1 ; Cl] | A | 2.78 | 567.2 |
| I-1-51 | 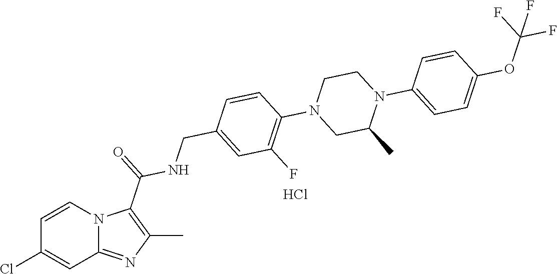 [SMILES: Cc1nc2cc(Cl)ccn2c1C(=O)NCc1ccc(N2CCN(c3ccc(OC(F)(F)F)cc3)[C@@H](C)C2)c(F)c1 ; Cl] | B | 3.27 | 610.15 |
| I-1-52 | 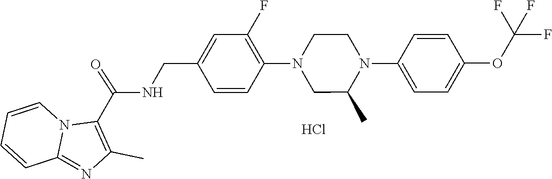 [SMILES: Cc1nc2ccccn2c1C(=O)NCc1ccc(N2CCN(c3ccc(OC(F)(F)F)cc3)[C@@H](C)C2)c(F)c1 ; Cl] | B | 2.855 | 542.2 |
| I-1-53 | 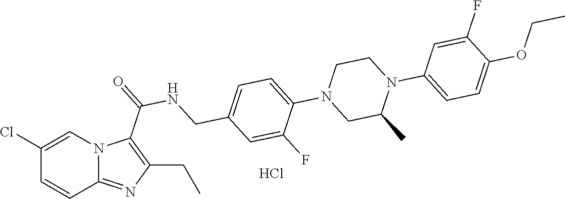 [SMILES: CCOc1ccc(N2CCN(c3ccc(CNC(=O)c4c(CC)nc5ccc(Cl)cn45)cc3F)C[C@@H]2C)cc1F ; Cl] | A | 2.371 | 568.3 |
| I-1-54 | 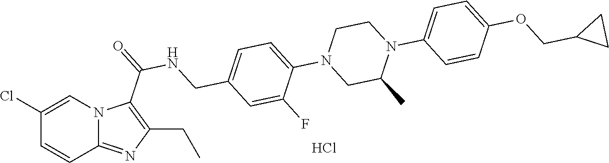 [SMILES: CCc1nc2ccc(Cl)cn2c1C(=O)NCc1ccc(N2CCN(c3ccc(OCC4CC4)cc3)[C@@H](C)C2)c(F)c1 ; Cl] | A | 1.898 | 576.3 |

TABLE 10

| | | | | |
|---|---|---|---|---|
| I-1-55 | 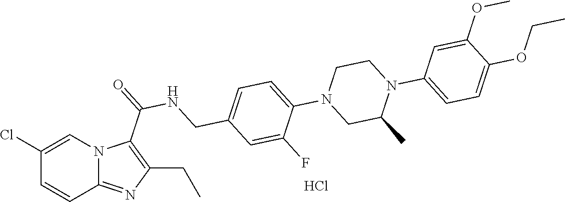 [SMILES: CCOc1ccc(N2CCN(c3ccc(CNC(=O)c4c(CC)nc5ccc(Cl)cn45)cc3F)C[C@@H]2C)cc1OC ; Cl] | A | 1.678 | 580.3 |
| I-1-56 | 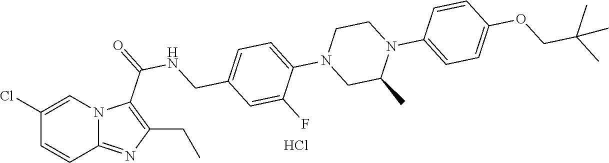 [SMILES: CCc1nc2ccc(Cl)cn2c1C(=O)NCc1ccc(N2CCN(c3ccc(OCC(C)(C)C)cc3)[C@@H](C)C2)c(F)c1 ; Cl] | A | 2.159 | 604.2 |
| I-1-57 | 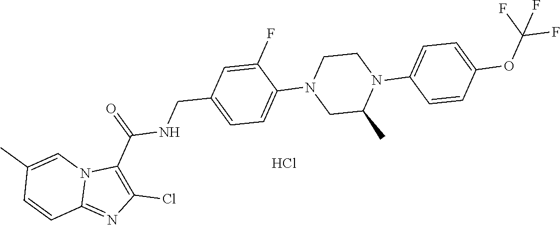 [SMILES: Cc1ccc2nc(Cl)c(C(=O)NCc3ccc(N4CCN(c5ccc(OC(F)(F)F)cc5)[C@@H](C)C4)c(F)c3)n2c1 ; Cl] | A | 3.23 | 576 |
| I-1-58 | 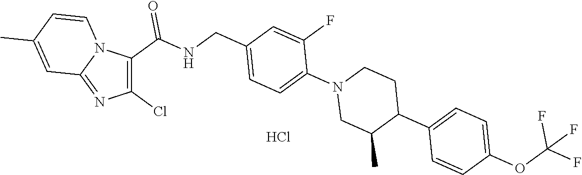 [SMILES: Cc1ccn2c(C(=O)NCc3ccc(N4CCC(c5ccc(OC(F)(F)F)cc5)[C@@H](C)C4)c(F)c3)c(Cl)nc2c1 ; Cl] | A | 3.2 | 576 |
| I-1-59 | 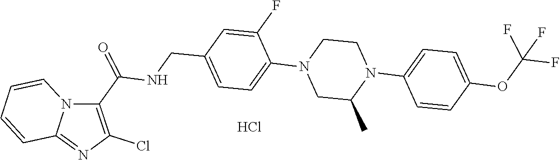 [SMILES: C[C@H]1CN(c2ccc(CNC(=O)c3c(Cl)nc4ccccn34)cc2F)CCN1c1ccc(OC(F)(F)F)cc1 ; Cl] | A | 3.09 | 562 |
| I-1-60 | 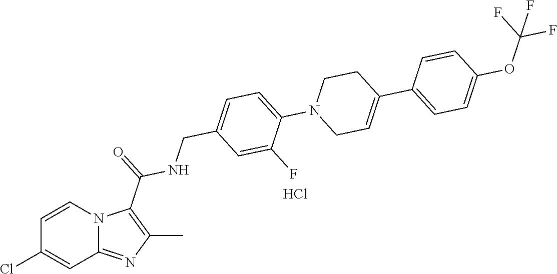 [SMILES: Cc1nc2cc(Cl)ccn2c1C(=O)NCc1ccc(N2CC=C(c3ccc(OC(F)(F)F)cc3)CC2)c(F)c1 ; Cl] | A | 3 | 573.2 |

TABLE 11

| | | | | |
|---|---|---|---|---|
| I-1-61 | 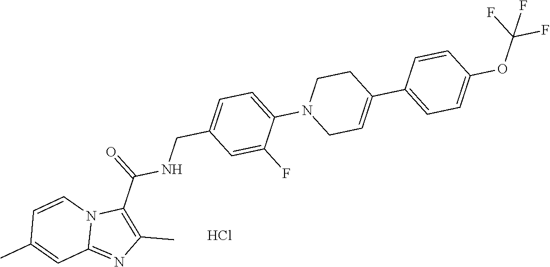 [SMILES: Cc1ccn2c(C(=O)NCc3ccc(N4CC=C(c5ccc(OC(F)(F)F)cc5)CC4)c(F)c3)c(C)nc2c1 ; Cl] | A | 2.21 | 539.2 |
| I-1-62 | 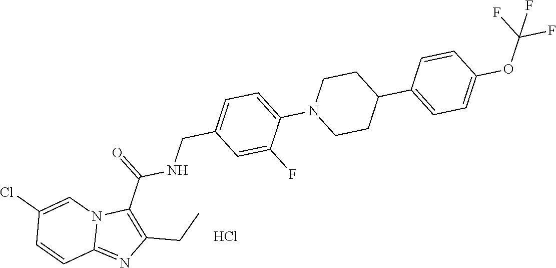 [SMILES: CCc1nc2ccc(Cl)cn2c1C(=O)NCc1ccc(N2CCC(c3ccc(OC(F)(F)F)cc3)CC2)c(F)c1 ; Cl] | A | 2.931 | 575.2 |
| I-1-63 | 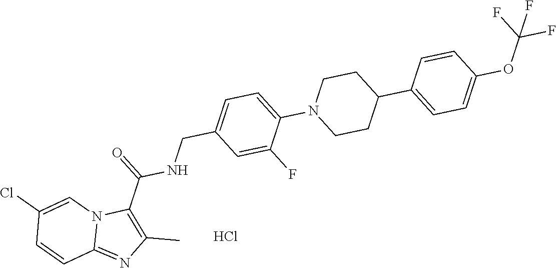 [SMILES: Cc1nc2ccc(Cl)cn2c1C(=O)NCc1ccc(N2CCC(c3ccc(OC(F)(F)F)cc3)CC2)c(F)c1 ; Cl] | A | 2.825 | 561.1 |
| I-1-64 | 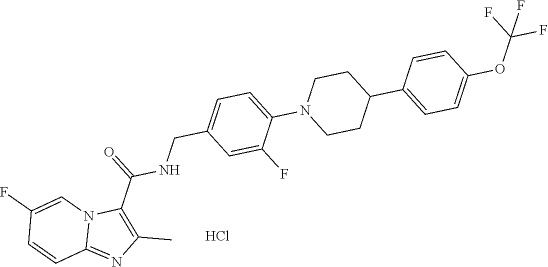 [SMILES: Cc1nc2ccc(F)cn2c1C(=O)NCc1ccc(N2CCC(c3ccc(OC(F)(F)F)cc3)CC2)c(F)c1 ; Cl] | A | 2.574 | 545.2 |
| I-1-65 | 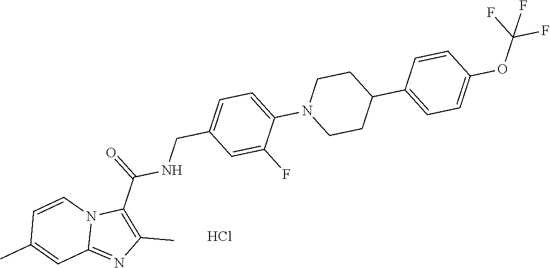 [SMILES: Cc1ccn2c(C(=O)NCc3ccc(N4CCC(c5ccc(OC(F)(F)F)cc5)CC4)c(F)c3)c(C)nc2c1 ; Cl] | A | 2.174 | 541.3 |
| I-1-66 | 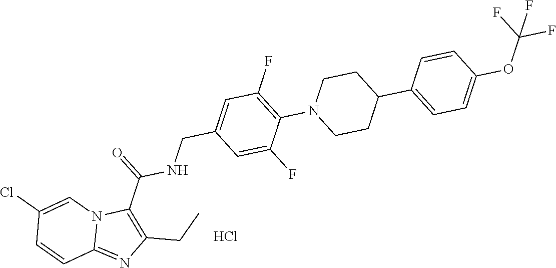 [SMILES: CCc1nc2ccc(Cl)cn2c1C(=O)NCc1cc(F)c(N2CCC(c3ccc(OC(F)(F)F)cc3)CC2)c(F)c1 ; Cl] | A | 3.19 | 593.2 |

TABLE 12

| | | | | |
|---|---|---|---|---|
| I-1-67 | 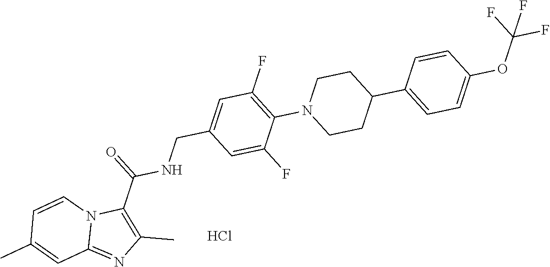 [SMILES: Cc1ccn2c(C(=O)NCc3cc(F)c(N4CCC(c5ccc(OC(F)(F)F)cc5)CC4)c(F)c3)c(C)nc2c1 ; Cl] | A | 2.32 | 559.2 |
| I-1-68 | 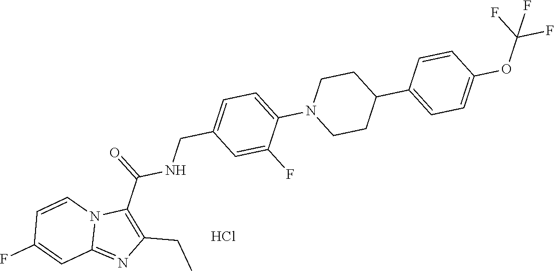 [SMILES: CCc1nc2cc(F)ccn2c1C(=O)NCc1ccc(N2CCC(c3ccc(OC(F)(F)F)cc3)CC2)c(F)c1 ; Cl] | A | 2.55 | 559.2 |
| I-1-69 | 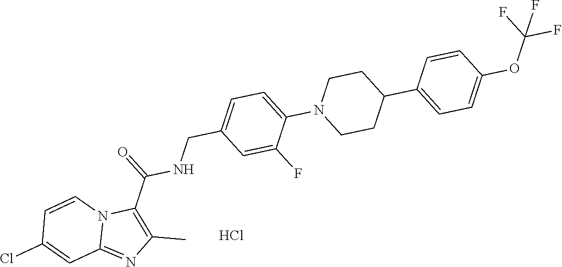 [SMILES: Cc1nc2cc(Cl)ccn2c1C(=O)NCc1ccc(N2CCC(c3ccc(OC(F)(F)F)cc3)CC2)c(F)c1 ; Cl] | A | 2.743 | 561.2 |
| I-1-70 | 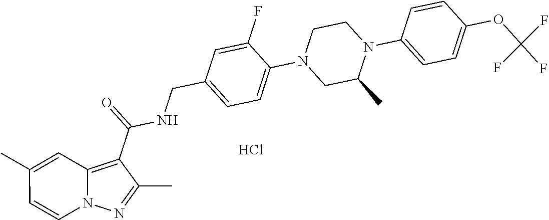 [SMILES: Cc1ccn2nc(C)c(C(=O)NCc3ccc(N4CCN(c5ccc(OC(F)(F)F)cc5)[C@@H](C)C4)c(F)c3)c2c1 ; Cl] | A | 2.94 | 556.25 |
| I-1-71 | 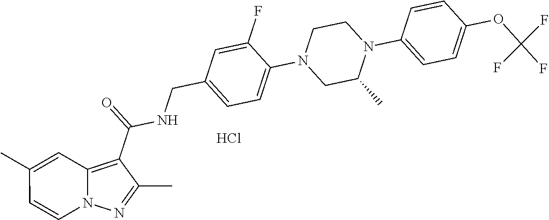 [SMILES: Cc1ccn2nc(C)c(C(=O)NCc3ccc(N4CCN(c5ccc(OC(F)(F)F)cc5)[C@H](C)C4)c(F)c3)c2c1 ; Cl] | A | 2.94 | 556.25 |
| I-1-72 | 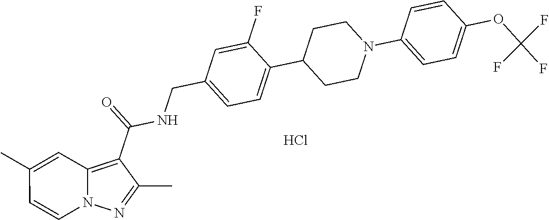 [SMILES: Cc1ccn2nc(C)c(C(=O)NCc3ccc(C4CCN(c5ccc(OC(F)(F)F)cc5)CC4)c(F)c3)c2c1 ; Cl] | A | 2.92 | 541.25 |

TABLE 13

| | | | | |
|---|---|---|---|---|
| I-1-73 | 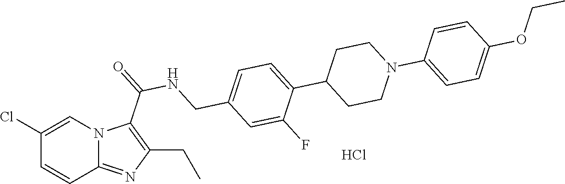 [SMILES: CCOc1ccc(N2CCC(c3ccc(CNC(=O)c4c(CC)nc5ccc(Cl)cn45)cc3F)CC2)cc1 ; Cl] | A | 1.676 | 535.3 |
| I-1-74 | 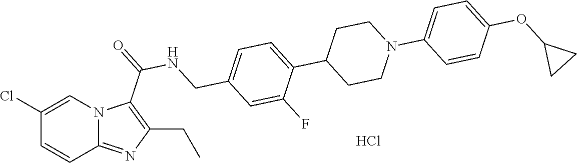 [SMILES: CCc1nc2ccc(Cl)cn2c1C(=O)NCc1ccc(C2CCN(c3ccc(OC4CC4)cc3)CC2)c(F)c1 ; Cl] | A | 1.748 | 547.2 |
| I-1-75 | 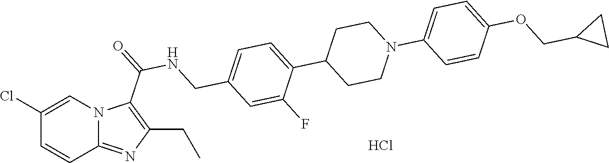 [SMILES: CCc1nc2ccc(Cl)cn2c1C(=O)NCc1ccc(C2CCN(c3ccc(OCC4CC4)cc3)CC2)c(F)c1 ; Cl] | A | 1.809 | 561.3 |
| I-1-76 | 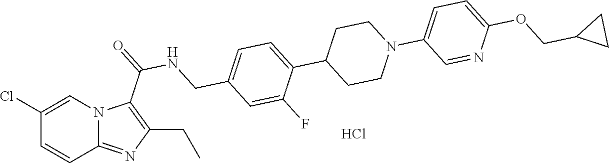 [SMILES: CCc1nc2ccc(Cl)cn2c1C(=O)NCc1ccc(C2CCN(c3ccc(OCC4CC4)nc3)CC2)c(F)c1 ; Cl] | A | 2.336 | 562.3 |
| I-1-77 | 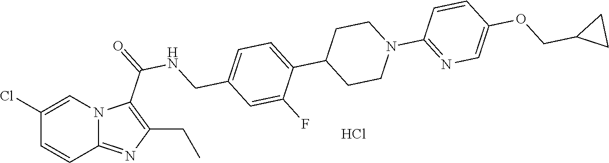 [SMILES: CCc1nc2ccc(Cl)cn2c1C(=O)NCc1ccc(C2CCN(c3ccc(OCC4CC4)cn3)CC2)c(F)c1 ; Cl] | A | 1.802 | 562.3 |
| I-1-78 | 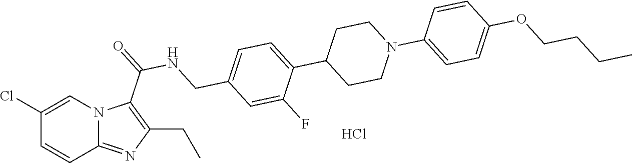 [SMILES: CCCCOc1ccc(N2CCC(c3ccc(CNC(=O)c4c(CC)nc5ccc(Cl)cn45)cc3F)CC2)cc1 ; Cl] | A | 1.976 | 563.3 |

TABLE 14

| | | | | |
|---|---|---|---|---|
| I-1-79 | 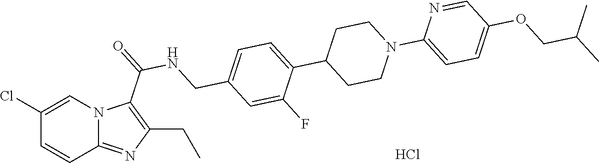 [SMILES: CCc1nc2ccc(Cl)cn2c1C(=O)NCc1ccc(C2CCN(c3ccc(OCC(C)C)cn3)CC2)c(F)c1 ; Cl] | A | 1.961 | 564.2 |
| I-1-80 | 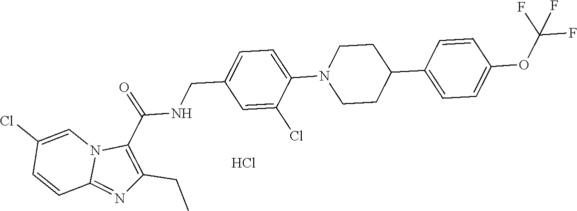 [SMILES: CCc1nc2ccc(Cl)cn2c1C(=O)NCc1ccc(N2CCC(c3ccc(OC(F)(F)F)cc3)CC2)c(Cl)c1 ; Cl] | A | 3.208 | 591.1 |
| I-1-81 | 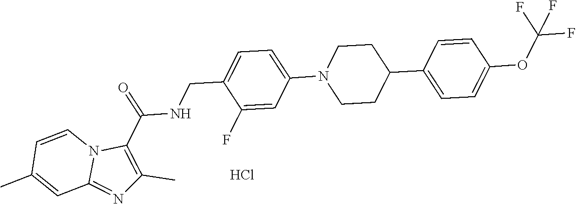 [SMILES: Cc1ccn2c(C(=O)NCc3ccc(N4CCC(c5ccc(OC(F)(F)F)cc5)CC4)cc3F)c(C)nc2c1 ; Cl] | A | 2.245 | 541.1 |
| I-1-82 | 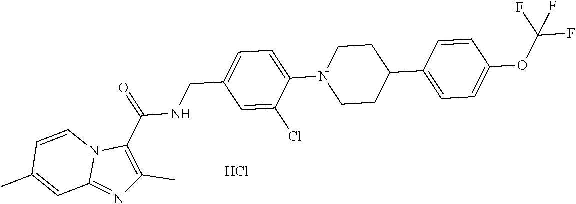 [SMILES: Cc1ccn2c(C(=O)NCc3ccc(N4CCC(c5ccc(OC(F)(F)F)cc5)CC4)c(Cl)c3)c(C)nc2c1 ; Cl] | A | 2.418 | 557.1 |
| I-1-83 | 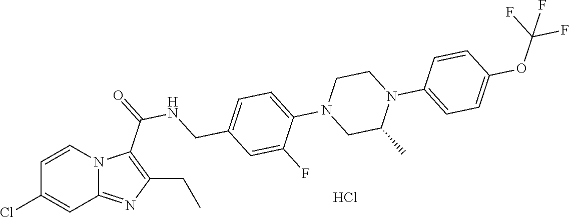 [SMILES: CCc1nc2cc(Cl)ccn2c1C(=O)NCc1ccc(N2CCN(c3ccc(OC(F)(F)F)cc3)[C@H](C)C2)c(F)c1 ; Cl] | A | 2.887 | 590.1 |
| I-1-84 | 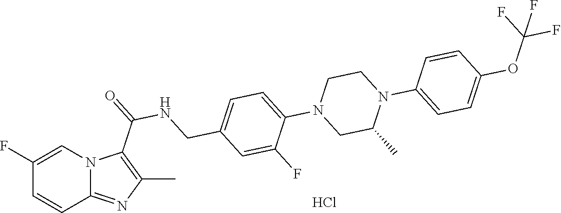 [SMILES: Cc1nc2ccc(F)cn2c1C(=O)NCc1ccc(N2CCN(c3ccc(OC(F)(F)F)cc3)[C@H](C)C2)c(F)c1 ; Cl] | A | 2.604 | 560.2 |

TABLE 15

| | | | | |
|---|---|---|---|---|
| I-1-85 | 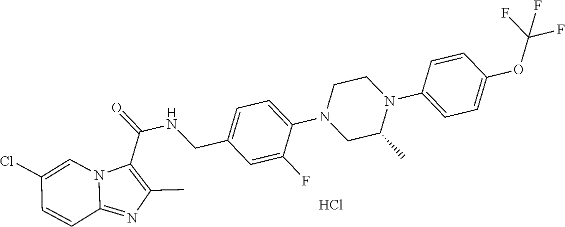 [SMILES: Cc1nc2ccc(Cl)cn2c1C(=O)NCc1ccc(N2CCN(c3ccc(OC(F)(F)F)cc3)[C@H](C)C2)c(F)c1 ; Cl] | A | 2.852 | 576.2 |
| I-1-86 | 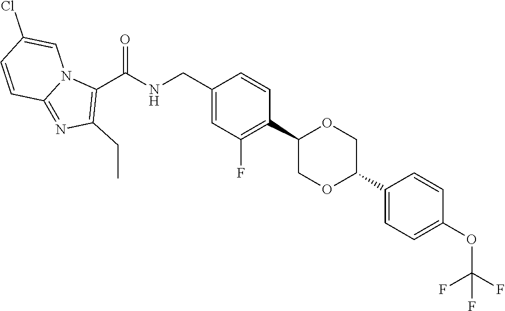 [SMILES: CCc1nc2ccc(Cl)cn2c1C(=O)NCc1ccc([C@@H]2CO[C@@H](c3ccc(OC(F)(F)F)cc3)CO2)c(F)c1] | B | 2.99 | 578.1 |
| I-1-87 | 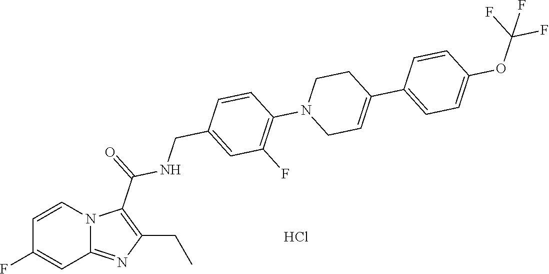 [SMILES: CCc1nc2cc(F)ccn2c1C(=O)NCc1ccc(N2CC=C(c3ccc(OC(F)(F)F)cc3)CC2)c(F)c1 ; Cl] | A | 2.568 | 557.2 |
| I-1-88 | 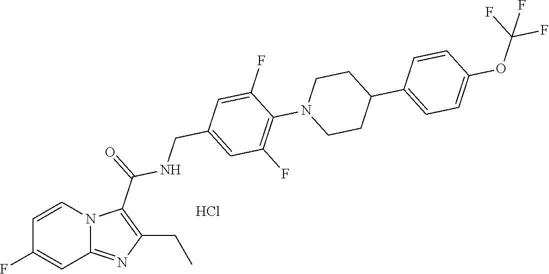 [SMILES: CCc1nc2cc(F)ccn2c1C(=O)NCc1cc(F)c(N2CCC(c3ccc(OC(F)(F)F)cc3)CC2)c(F)c1 ; Cl] | A | 2.819 | 577.2 |
| I-1-89 | 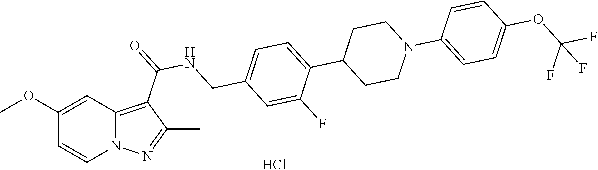 [SMILES: COc1ccn2nc(C)c(C(=O)NCc3ccc(C4CCN(c5ccc(OC(F)(F)F)cc5)CC4)c(F)c3)c2c1 ; Cl] | A | 2.824 | 557.2 |
| I-1-90 | 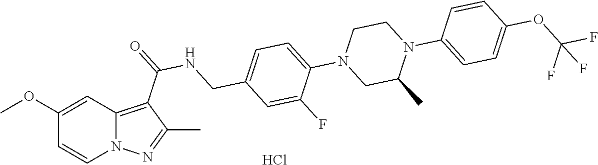 [SMILES: COc1ccn2nc(C)c(C(=O)NCc3ccc(N4CCN(c5ccc(OC(F)(F)F)cc5)[C@@H](C)C4)c(F)c3)c2c1 ; Cl] | A | 2.868 | 572.2 |

TABLE 16

| | | | | |
|---|---|---|---|---|
| I-1-91 | 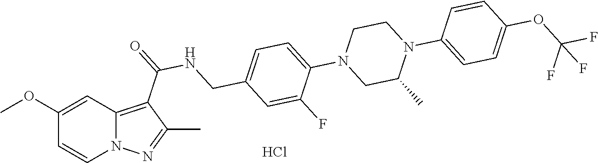 [SMILES: COc1ccn2nc(C)c(C(=O)NCc3ccc(N4CCN(c5ccc(OC(F)(F)F)cc5)[C@H](C)C4)c(F)c3)c2c1 ; Cl] | A | 2.868 | 572.2 |
| I-1-92 | 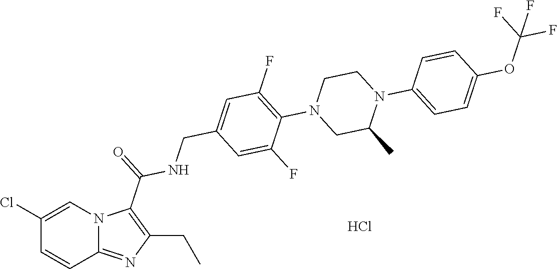 [SMILES: CCc1nc2ccc(Cl)cn2c1C(=O)NCc1cc(F)c(N2CCN(c3ccc(OC(F)(F)F)cc3)[C@@H](C)C2)c(F)c1 ; Cl] | A | 3.113 | 608.1 |
| I-1-93 | 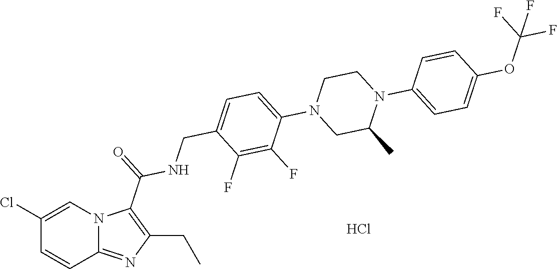 [SMILES: CCc1nc2ccc(Cl)cn2c1C(=O)NCc1ccc(N2CCN(c3ccc(OC(F)(F)F)cc3)[C@@H](C)C2)c(F)c1F ; Cl] | A | 3.041 | 608.1 |
| I-1-94 | 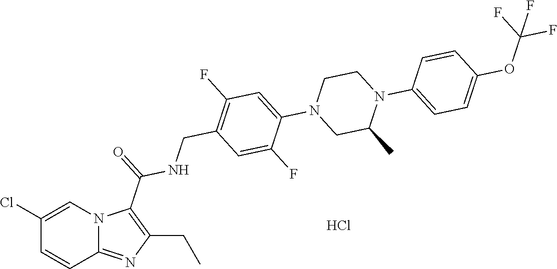 [SMILES: CCc1nc2ccc(Cl)cn2c1C(=O)NCc1cc(F)c(N2CCN(c3ccc(OC(F)(F)F)cc3)[C@@H](C)C2)cc1F ; Cl] | A | 3.048 | 608.1 |
| I-1-95 | 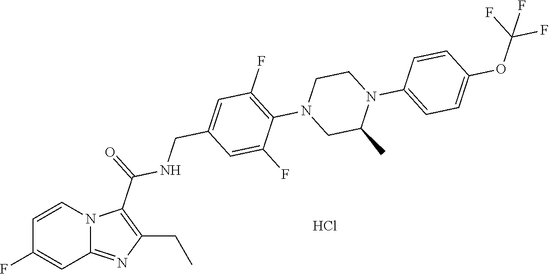 [SMILES: CCc1nc2cc(F)ccn2c1C(=O)NCc1cc(F)c(N2CCN(c3ccc(OC(F)(F)F)cc3)[C@@H](C)C2)c(F)c1 ; Cl] | A | 2.741 | 592.2 |
| I-1-96 | 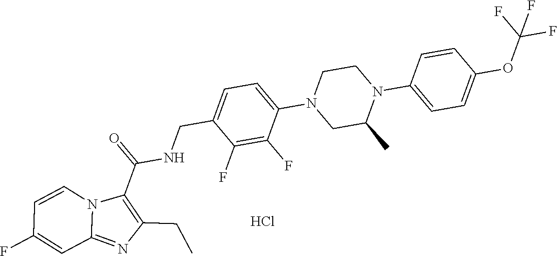 [SMILES: CCc1nc2cc(F)ccn2c1C(=O)NCc1ccc(N2CCN(c3ccc(OC(F)(F)F)cc3)[C@@H](C)C2)c(F)c1F ; Cl] | A | 2.677 | 592.2 |

TABLE 17

| | | | | |
|---|---|---|---|---|
| I-1-97 | 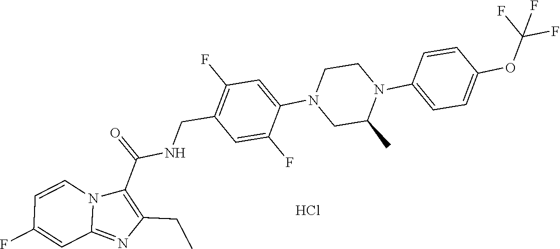 [SMILES: CCc1nc2cc(F)ccn2c1C(=O)NCc1cc(F)c(N2CCN(c3ccc(OC(F)(F)F)cc3)[C@@H](C)C2)cc1F ; Cl] | A | 2.682 | 592.2 |
| I-1-98 | 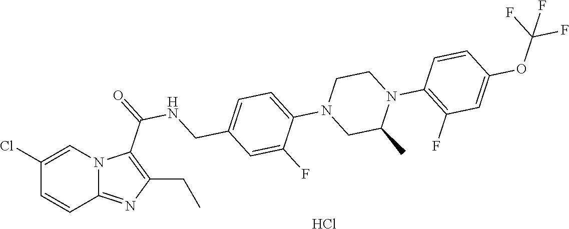 [SMILES: CCc1nc2ccc(Cl)cn2c1C(=O)NCc1ccc(N2CCN(c3ccc(OC(F)(F)F)cc3F)[C@@H](C)C2)c(F)c1 ; Cl] | B | 3.276 | 608.2 |
| I-1-99 | 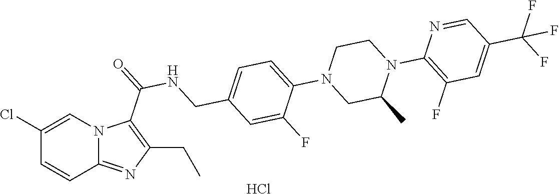 [SMILES: CCc1nc2ccc(Cl)cn2c1C(=O)NCc1ccc(N2CCN(c3ncc(C(F)(F)F)cc3F)[C@@H](C)C2)c(F)c1 ; Cl] | B | 3.267 | 593.1 |
| I-1-100 | 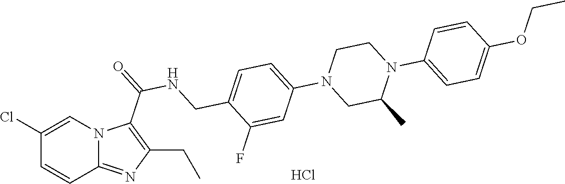 [SMILES: CCOc1ccc(N2CCN(c3ccc(CNC(=O)c4c(CC)nc5ccc(Cl)cn45)c(F)c3)C[C@@H]2C)cc1 ; Cl] | B | 2.95 | 550 |
| I-1-101 | 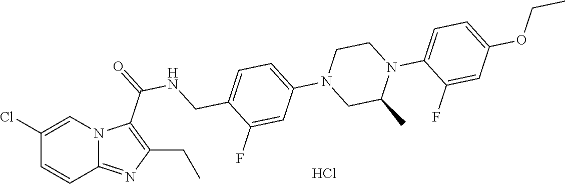 [SMILES: CCOc1ccc(N2CCN(c3ccc(CNC(=O)c4c(CC)nc5ccc(Cl)cn45)c(F)c3)C[C@@H]2C)c(F)c1 ; Cl] | B | 3.08 | 568 |
| I-1-102 | 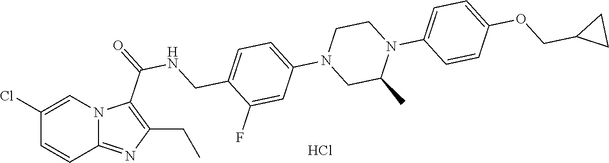 [SMILES: CCc1nc2ccc(Cl)cn2c1C(=O)NCc1ccc(N2CCN(c3ccc(OCC4CC4)cc3)[C@@H](C)C2)cc1F ; Cl] | B | 3.06 | 576 |

TABLE 18

| | | | | |
|---|---|---|---|---|
| I-1-103 | 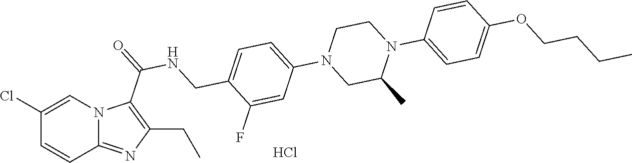 [SMILES: CCCCOc1ccc(N2CCN(c3ccc(CNC(=O)c4c(CC)nc5ccc(Cl)cn45)c(F)c3)C[C@@H]2C)cc1 ; Cl] | B | 3.33 | 578 |
| I-1-104 | 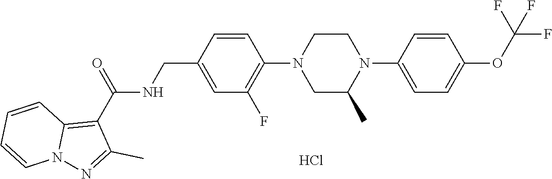 [SMILES: Cc1nn2ccccc2c1C(=O)NCc1ccc(N2CCN(c3ccc(OC(F)(F)F)cc3)[C@@H](C)C2)c(F)c1 ; Cl] | A | 2.79 | 542.1 |
| I-1-105 | 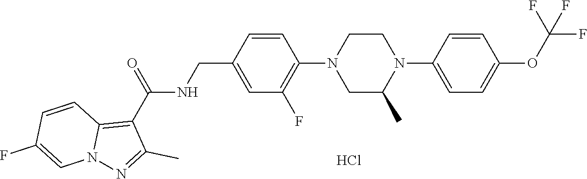 [SMILES: Cc1nn2cc(F)ccc2c1C(=O)NCc1ccc(N2CCN(c3ccc(OC(F)(F)F)cc3)[C@@H](C)C2)c(F)c1 ; Cl] | A | 2.902 | 560.1 |
| I-1-106 | 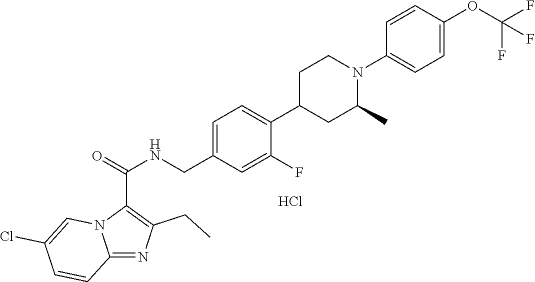 [SMILES: CCc1nc2ccc(Cl)cn2c1C(=O)NCc1ccc(C2CCN(c3ccc(OC(F)(F)F)cc3)[C@@H](C)C2)c(F)c1 ; Cl] | A | 2.77 | 589.15 |
| I-1-107 | 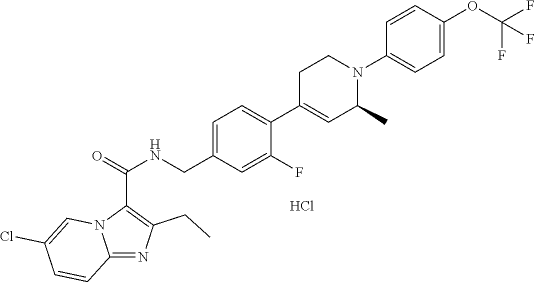 [SMILES: CCc1nc2ccc(Cl)cn2c1C(=O)NCc1ccc(C2=C[C@H](C)N(c3ccc(OC(F)(F)F)cc3)CC2)c(F)c1 ; Cl] | A | 3.27 | 587.15 |
| I-1-108 | 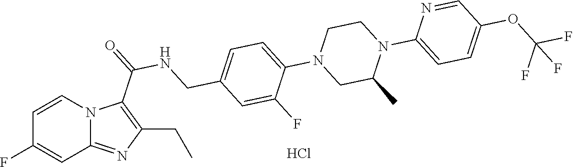 [SMILES: CCc1nc2cc(F)ccn2c1C(=O)NCc1ccc(N2CCN(c3ccc(OC(F)(F)F)cn3)[C@@H](C)C2)c(F)c1 ; Cl] | A | 2.51 | 575 |

TABLE 19

| | | | | |
|---|---|---|---|---|
| I-1-109 | 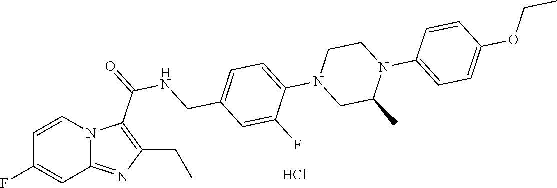 [SMILES: CCOc1ccc(N2CCN(c3ccc(CNC(=O)c4c(CC)nc5cc(F)ccn45)cc3F)C[C@@H]2C)cc1 ; Cl] | A | 1.57 | 534 |
| I-1-110 | 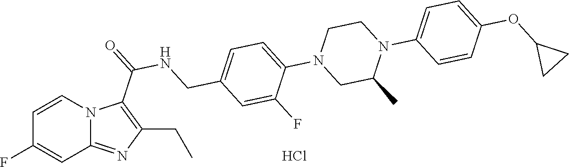 [SMILES: CCc1nc2cc(F)ccn2c1C(=O)NCc1ccc(N2CCN(c3ccc(OC4CC4)cc3)[C@@H](C)C2)c(F)c1 ; Cl] | A | 1.65 | 546 |
| I-1-111 | 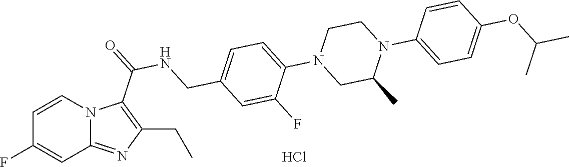 [SMILES: CCc1nc2cc(F)ccn2c1C(=O)NCc1ccc(N2CCN(c3ccc(OC(C)C)cc3)[C@@H](C)C2)c(F)c1 ; Cl] | A | 1.67 | 548 |
| I-1-112 | 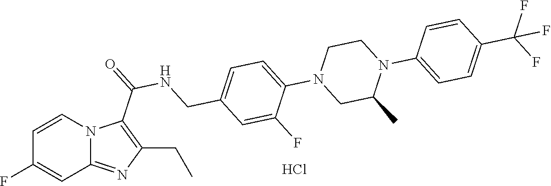 [SMILES: CCc1nc2cc(F)ccn2c1C(=O)NCc1ccc(N2CCN(c3ccc(C(F)(F)F)cc3)[C@@H](C)C2)c(F)c1 ; Cl] | A | 2.61 | 558 |
| I-1-113 | 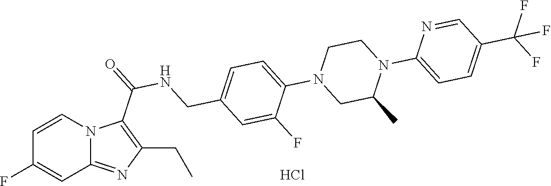 [SMILES: CCc1nc2cc(F)ccn2c1C(=O)NCc1ccc(N2CCN(c3ccc(C(F)(F)F)cn3)[C@@H](C)C2)c(F)c1 ; Cl] | A | 2.47 | 559 |
| I-1-114 | 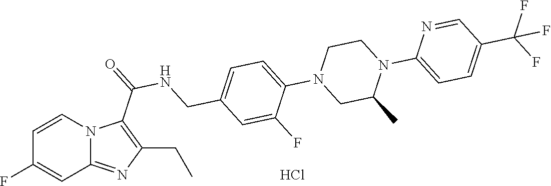 [SMILES: CCc1nc2cc(F)ccn2c1C(=O)NCc1ccc(N2CCN(c3ccc(C(F)(F)F)cn3)[C@@H](C)C2)c(F)c1 ; Cl] | A | 1.89 | 562 |

TABLE 20

| | | | | |
|---|---|---|---|---|
| I-1-115 | 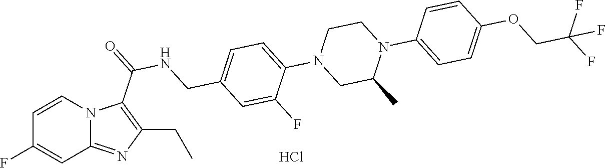 [SMILES: CCc1nc2cc(F)ccn2c1C(=O)NCc1ccc(N2CCN(c3ccc(OCC(F)(F)F)cc3)[C@@H](C)C2)c(F)c1 ; Cl] | A | 1.89 | 588 |
| I-1-116 | 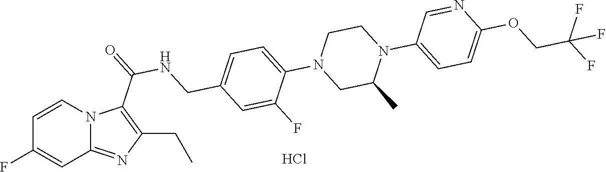 [SMILES: CCc1nc2cc(F)ccn2c1C(=O)NCc1ccc(N2CCN(c3ccc(OCC(F)(F)F)nc3)[C@@H](C)C2)c(F)c1 ; Cl] | A | 2.36 | 589 |
| I-1-117 | 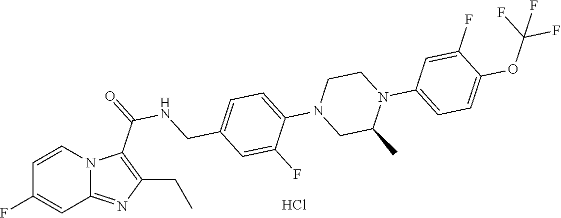 [SMILES: CCc1nc2cc(F)ccn2c1C(=O)NCc1ccc(N2CCN(c3ccc(OC(F)(F)F)c(F)c3)[C@@H](C)C2)c(F)c1 ; Cl] | A | 2.69 | 592 |
| I-1-118 | 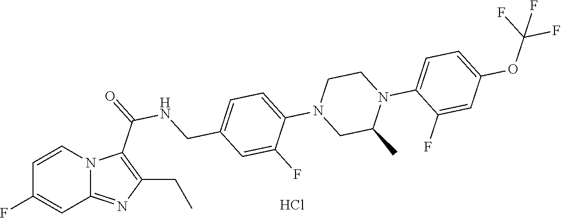 [SMILES: CCc1nc2cc(F)ccn2c1C(=O)NCc1ccc(N2CCN(c3ccc(OC(F)(F)F)cc3F)[C@@H](C)C2)c(F)c1 ; Cl] | A | 2.68 | 592 |
| I-1-119 | 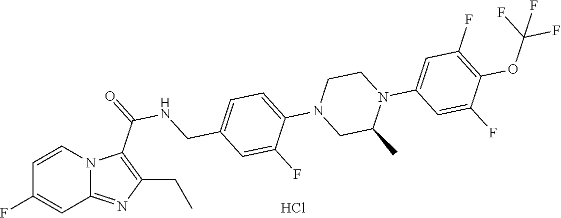 [SMILES: CCc1nc2cc(F)ccn2c1C(=O)NCc1ccc(N2CCN(c3cc(F)c(OC(F)(F)F)c(F)c3)[C@@H](C)C2)c(F)c1 ; Cl] | A | 2.78 | 610 |
| I-1-120 | 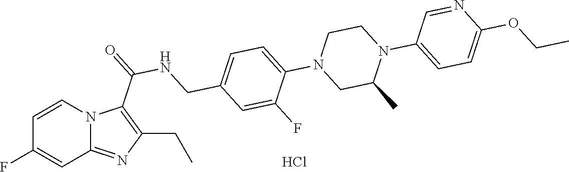 [SMILES: CCOc1ccc(N2CCN(c3ccc(CNC(=O)c4c(CC)nc5cc(F)ccn45)cc3F)C[C@@H]2C)cn1 ; Cl] | A | 1.91 | 535 |

TABLE 21

| | | | | |
|---|---|---|---|---|
| I-1-121 | 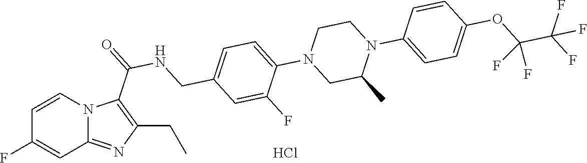 [SMILES: CCc1nc2cc(F)ccn2c1C(=O)NCc1ccc(N2CCN(c3ccc(OC(F)(F)C(F)(F)F)cc3)[C@@H](C)C2)c(F)c1 ; Cl] | A | 2.8 | 624 |
| I-1-122 | 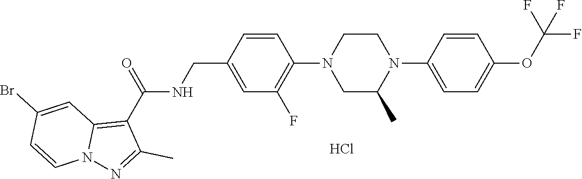 [SMILES: Cc1nn2ccc(Br)cc2c1C(=O)NCc1ccc(N2CCN(c3ccc(OC(F)(F)F)cc3)[C@@H](C)C2)c(F)c1 ; Cl] | A | 3.078 | 620 |
| I-1-123 | 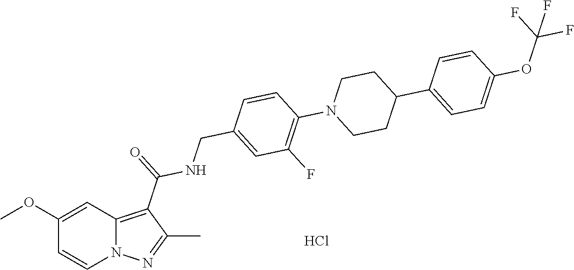 [SMILES: COc1ccn2nc(C)c(C(=O)NCc3ccc(N4CCC(c5ccc(OC(F)(F)F)cc5)CC4)c(F)c3)c2c1 ; Cl] | A | 2.882 | 557.2 |
| I-1-124 | 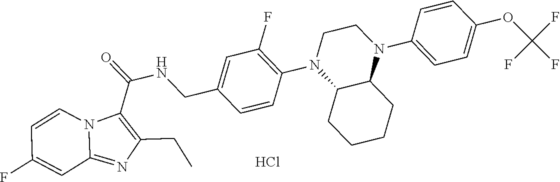 [SMILES: CCc1nc2cc(F)ccn2c1C(=O)NCc1ccc(N2CCN(c3ccc(OC(F)(F)F)cc3)[C@H]3CCCC[C@@H]32)c(F)c1 ; Cl] | B | 3.16 | 614.2 |
| I-1-125 | 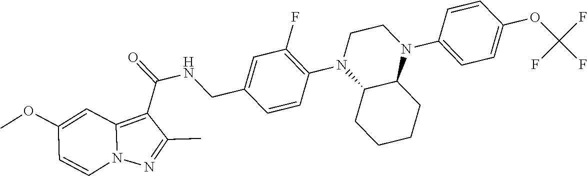 [SMILES: COc1ccn2nc(C)c(C(=O)NCc3ccc(N4CCN(c5ccc(OC(F)(F)F)cc5)[C@H]5CCCC[C@@H]54)c(F)c3)c2c1] | B | 3.06 | 612.2 |
| I-1-126 | 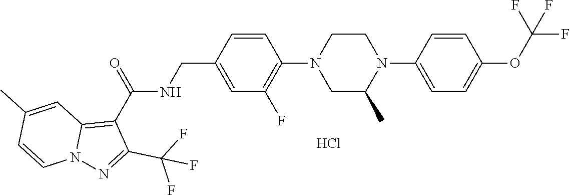 [SMILES: Cc1ccn2nc(C(F)(F)F)c(C(=O)NCc3ccc(N4CCN(c5ccc(OC(F)(F)F)cc5)[C@@H](C)C4)c(F)c3)c2c1 ; Cl] | A | 3.151 | 610.2 |

TABLE 22

| | | | | |
|---|---|---|---|---|
| I-1-127 | 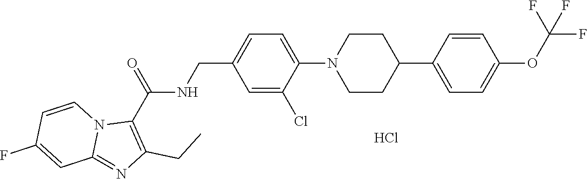 [SMILES: CCc1nc2cc(F)ccn2c1C(=O)NCc1ccc(N2CCC(c3ccc(OC(F)(F)F)cc3)CC2)c(Cl)c1 ; Cl] | A | 2.842 | 575.2 |
| I-1-128 | 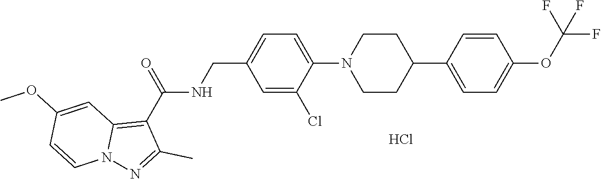 [SMILES: COc1ccn2nc(C)c(C(=O)NCc3ccc(N4CCC(c5ccc(OC(F)(F)F)cc5)CC4)c(Cl)c3)c2c1 ; Cl] | A | 3.1 | 573.2 |
| I-1-129 | 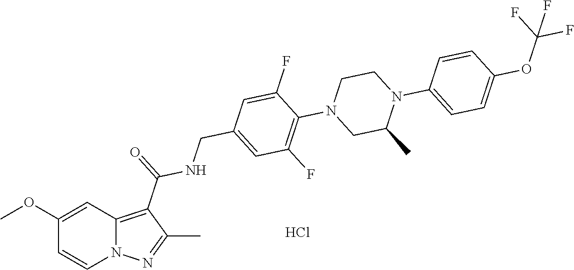 [SMILES: COc1ccn2nc(C)c(C(=O)NCc3cc(F)c(N4CCN(c5ccc(OC(F)(F)F)cc5)[C@@H](C)C4)c(F)c3)c2c1 ; Cl] | A | 2.985 | 590.2 |
| I-1-130 | 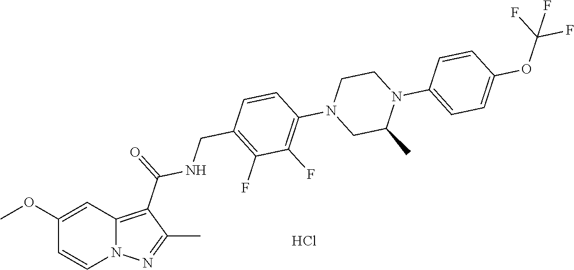 [SMILES: COc1ccn2nc(C)c(C(=O)NCc3ccc(N4CCN(c5ccc(OC(F)(F)F)cc5)[C@@H](C)C4)c(F)c3F)c2c1 ; Cl] | A | 2.964 | 590.2 |
| I-1-131 | 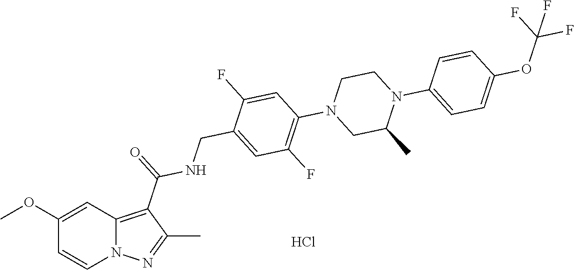 [SMILES: COc1ccn2nc(C)c(C(=O)NCc3cc(F)c(N4CCN(c5ccc(OC(F)(F)F)cc5)[C@@H](C)C4)cc3F)c2c1 ; Cl] | A | 2.961 | 590.2 |
| I-1-132 | 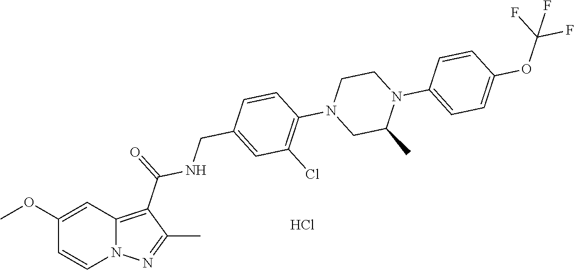 [SMILES: COc1ccn2nc(C)c(C(=O)NCc3ccc(N4CCN(c5ccc(OC(F)(F)F)cc5)[C@@H](C)C4)c(Cl)c3)c2c1 ; Cl] | A | 3.022 | 588.1 |

TABLE 23

| | | | | |
|---|---|---|---|---|
| I-1-133 | 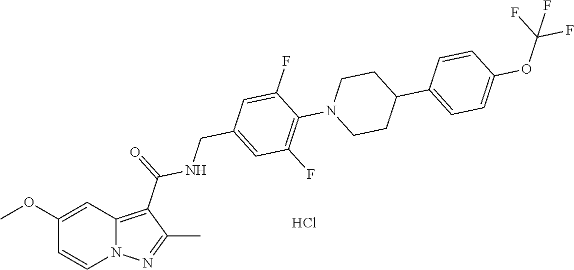 [SMILES: COc1ccn2nc(C)c(C(=O)NCc3cc(F)c(N4CCC(c5ccc(OC(F)(F)F)cc5)CC4)c(F)c3)c2c1 ; Cl] | A | 3.111 | 575.2 |
| I-1-134 | 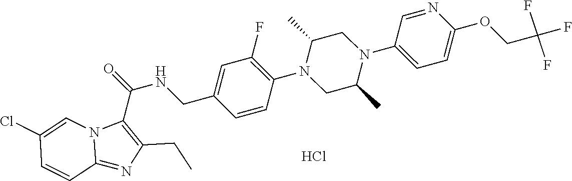 [SMILES: CCc1nc2ccc(Cl)cn2c1C(=O)NCc1ccc(N2C[C@H](C)N(c3ccc(OCC(F)(F)F)nc3)C[C@H]2C)c(F)c1 ; Cl] | B | 3.07 | 619.2 |
| I-1-135 | 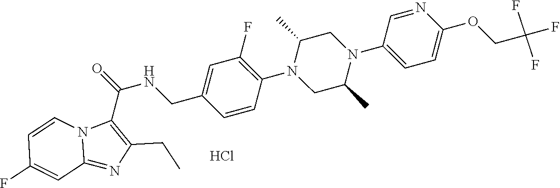 [SMILES: CCc1nc2cc(F)ccn2c1C(=O)NCc1ccc(N2C[C@H](C)N(c3ccc(OCC(F)(F)F)nc3)C[C@H]2C)c(F)c1 ; Cl] | B | 2.88 | 603.2 |
| I-1-136 | 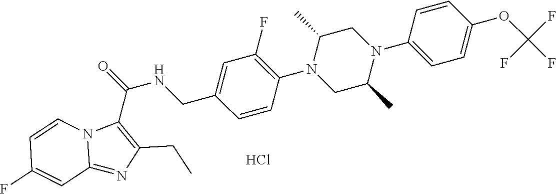 [SMILES: CCc1nc2cc(F)ccn2c1C(=O)NCc1ccc(N2C[C@H](C)N(c3ccc(OC(F)(F)F)cc3)C[C@H]2C)c(F)c1 ; Cl] | B | 3.13 | 588.2 |
| I-1-137 | 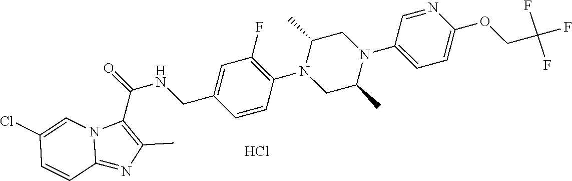 [SMILES: Cc1nc2ccc(Cl)cn2c1C(=O)NCc1ccc(N2C[C@H](C)N(c3ccc(OCC(F)(F)F)nc3)C[C@H]2C)c(F)c1 ; Cl] | B | 2.98 | 605.2 |
| I-1-138 | 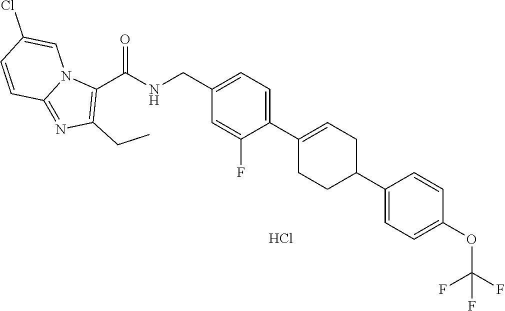 [SMILES: CCc1nc2ccc(Cl)cn2c1C(=O)NCc1ccc(C2=CCC(c3ccc(OC(F)(F)F)cc3)CC2)c(F)c1 ; Cl] | B | 3.4 | 572.1 |

TABLE 24

| | | | | |
|---|---|---|---|---|
| I-1-139 | 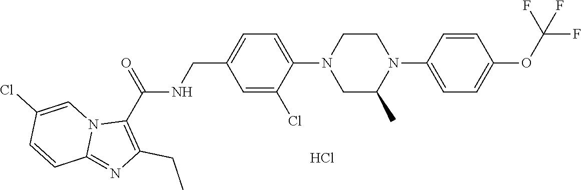 [SMILES: CCc1nc2ccc(Cl)cn2c1C(=O)NCc1ccc(N2CCN(c3ccc(OC(F)(F)F)cc3)[C@@H](C)C2)c(Cl)c1 ; Cl] | A | 3.09 | 606.1 |
| I-1-140 | 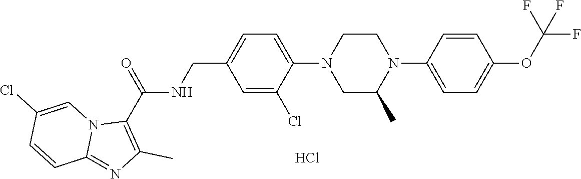 [SMILES: Cc1nc2ccc(Cl)cn2c1C(=O)NCc1ccc(N2CCN(c3ccc(OC(F)(F)F)cc3)[C@@H](C)C2)c(Cl)c1 ; Cl] | A | 3 | 592.1 |
| I-1-141 | 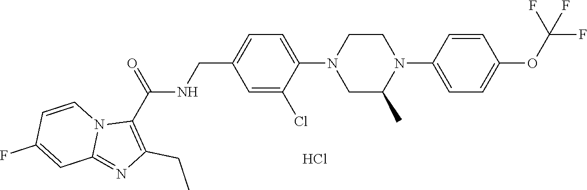 [SMILES: CCc1nc2cc(F)ccn2c1C(=O)NCc1ccc(N2CCN(c3ccc(OC(F)(F)F)cc3)[C@@H](C)C2)c(Cl)c1 ; Cl] | A | 2.7 | 590.1 |

TABLE 25

| | | LCMS | | MS |
|---|---|---|---|---|
| No. | Structure | method | RT | (M + H) |
| I-1-142 | 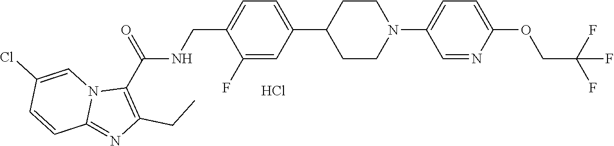 [SMILES: CCc1nc2ccc(Cl)cn2c1C(=O)NCc1ccc(C2CCN(c3ccc(OCC(F)(F)F)nc3)CC2)cc1F ; Cl] | B | 3.01 | 590 |
| I-1-143 | 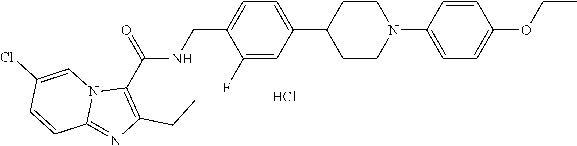 [SMILES: CCOc1ccc(N2CCC(c3ccc(CNC(=O)c4c(CC)nc5ccc(Cl)cn45)c(F)c3)CC2)cc1 ; Cl] | B | 3 | 535 |
| I-1-144 | 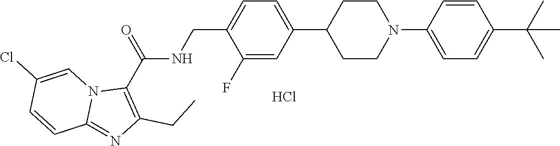 [SMILES: CCc1nc2ccc(Cl)cn2c1C(=O)NCc1ccc(C2CCN(c3ccc(C(C)(C)C)cc3)CC2)cc1F ; Cl] | B | 3.48 | 547 |
| I-1-145 | 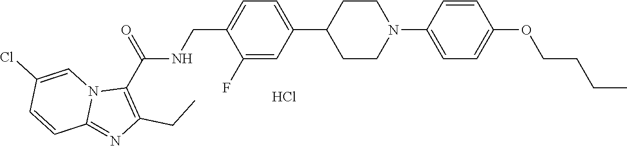 [SMILES: CCCCOc1ccc(N2CCC(c3ccc(CNC(=O)c4c(CC)nc5ccc(Cl)cn45)c(F)c3)CC2)cc1 ; Cl] | B | 3.36 | 563 |
| I-1-146 | 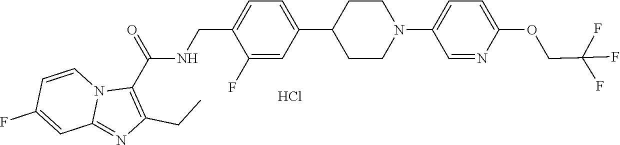 [SMILES: CCc1nc2cc(F)ccn2c1C(=O)NCc1ccc(C2CCN(c3ccc(OCC(F)(F)F)nc3)CC2)cc1F ; Cl] | B | 2.82 | 574 |
| I-1-147 | 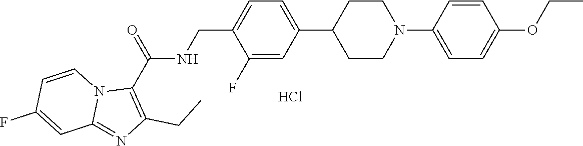 [SMILES: CCOc1ccc(N2CCC(c3ccc(CNC(=O)c4c(CC)nc5cc(F)ccn45)c(F)c3)CC2)cc1 ; Cl] | B | 2.79 | 519 |

TABLE 26

| | | | | |
|---|---|---|---|---|
| I-1-148 | 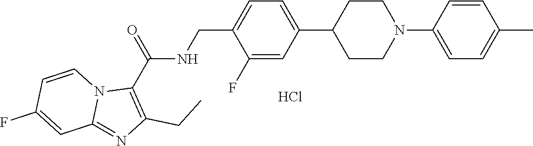 [SMILES: CCc1nc2cc(F)ccn2c1C(=O)NCc1ccc(C2CCN(c3ccc(C)cc3)CC2)cc1F ; Cl] | B | 2.92 | 489 |
| I-1-149 | 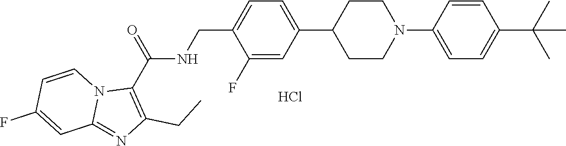 [SMILES: CCc1nc2cc(F)ccn2c1C(=O)NCc1ccc(C2CCN(c3ccc(C(C)(C)C)cc3)CC2)cc1F ; Cl] | B | 3.28 | 531 |
| I-1-150 | 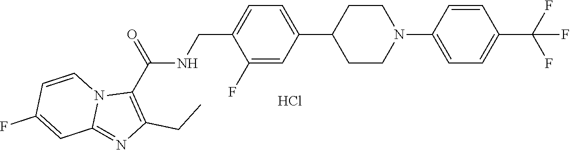 [SMILES: CCc1nc2cc(F)ccn2c1C(=O)NCc1ccc(C2CCN(c3ccc(C(F)(F)F)cc3)CC2)cc1F ; Cl] | B | 3 | 543 |
| I-1-151 | 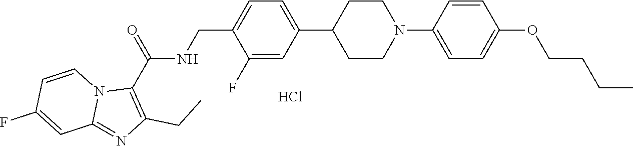 [SMILES: CCCCOc1ccc(N2CCC(c3ccc(CNC(=O)c4c(CC)nc5cc(F)ccn45)c(F)c3)CC2)cc1 ; Cl] | B | 3.16 | 547 |
| I-1-152 | 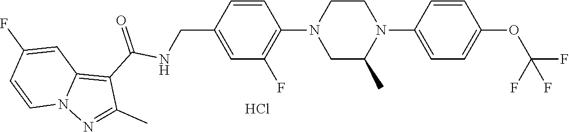 [SMILES: Cc1nn2ccc(F)cc2c1C(=O)NCc1ccc(N2CCN(c3ccc(OC(F)(F)F)cc3)[C@@H](C)C2)c(F)c1 ; Cl] | B | 2.9 | 560.15 |
| I-1-153 | 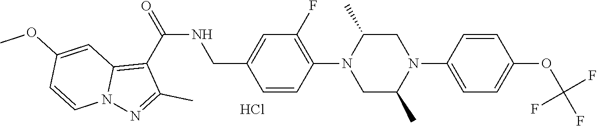 [SMILES: COc1ccn2nc(C)c(C(=O)NCc3ccc(N4C[C@H](C)N(c5ccc(OC(F)(F)F)cc5)C[C@H]4C)c(F)c3)c2c1 ; Cl] | B | 3.02 | 586.2 |

TABLE 27

| | | LCMS | | MS |
|---|---|---|---|---|
| No. | Structure | method | RT | (M + H) |
| I-2-1 | 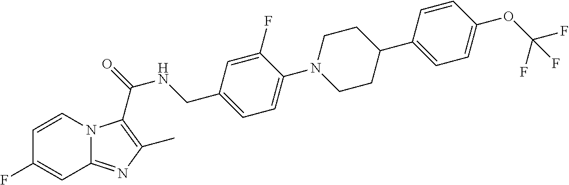 [SMILES: Cc1nc2cc(F)ccn2c1C(=O)NCc1ccc(N2CCC(c3ccc(OC(F)(F)F)cc3)CC2)c(F)c1] | A | 2.61 | 545.15 |
| I-2-2 | 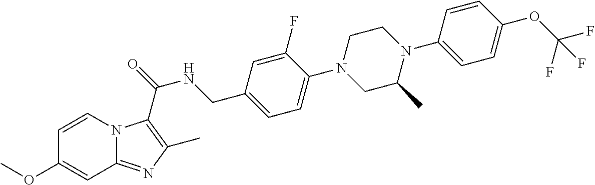 [SMILES: COc1ccn2c(C(=O)NCc3ccc(N4CCN(c5ccc(OC(F)(F)F)cc5)[C@@H](C)C4)c(F)c3)c(C)nc2c1] | A | 2.47 | 572.2 |
| I-2-3 | 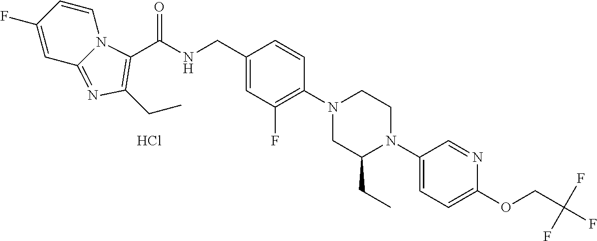 [SMILES: CCc1nc2cc(F)ccn2c1C(=O)NCc1ccc(N2CCN(c3ccc(OCC(F)(F)F)nc3)[C@@H](CC)C2)c(F)c1 ; Cl] | A | 2.63 | 603.2 |
| I-2-4 | 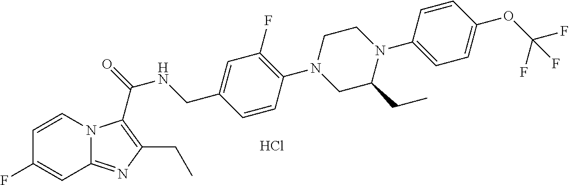 [SMILES: CCc1nc2cc(F)ccn2c1C(=O)NCc1ccc(N2CCN(c3ccc(OC(F)(F)F)cc3)[C@@H](CC)C2)c(F)c1 ; Cl] | A | 2.87 | 588.2 |
| I-2-5 | 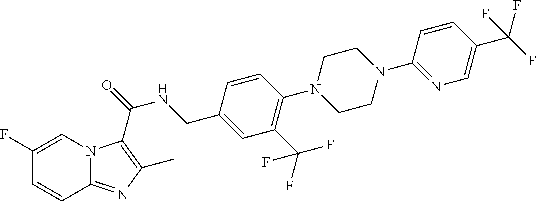 [SMILES: Cc1nc2ccc(F)cn2c1C(=O)NCc1ccc(N2CCN(c3ccc(C(F)(F)F)cn3)CC2)c(C(F)(F)F)c1] | A | 2.71 | 581.2 |
| I-2-6 | 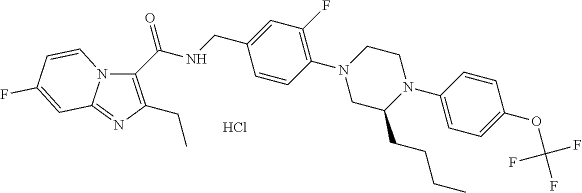 [SMILES: CCCC[C@H]1CN(c2ccc(CNC(=O)c3c(CC)nc4cc(F)ccn34)cc2F)CCN1c1ccc(OC(F)(F)F)cc1 ; Cl] | A | 3.15 | 616.2 |

TABLE 28

| | | | | |
|---|---|---|---|---|
| I-2-7 | 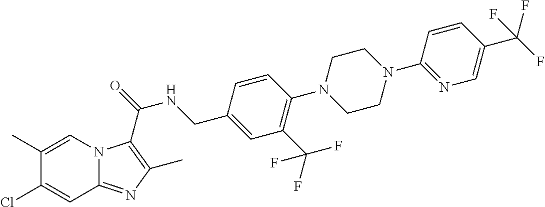 [SMILES: Cc1cn2c(C(=O)NCc3ccc(N4CCN(c5ccc(C(F)(F)F)cn5)CC4)c(C(F)(F)F)c3)c(C)nc2cc1Cl] | A | 2.86 | 611.15 |
| I-2-8 | 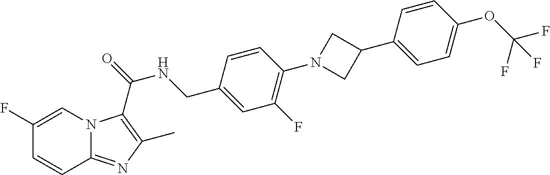 [SMILES: Cc1nc2ccc(F)cn2c1C(=O)NCc1ccc(N2CC(c3ccc(OC(F)(F)F)cc3)C2)c(F)c1] | A | 2.57 | 517.15 |
| I-2-9 | 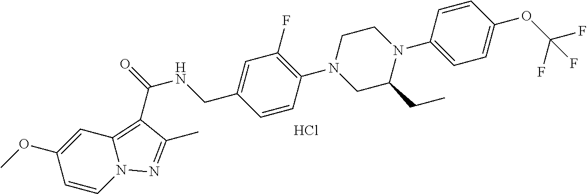 [SMILES: CC[C@H]1CN(c2ccc(CNC(=O)c3c(C)nn4ccc(OC)cc34)cc2F)CCN1c1ccc(OC(F)(F)F)cc1 ; Cl] | A | 3.01 | 586.25 |
| I-2-10 | 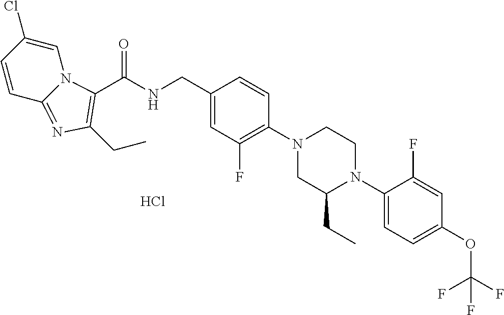 [SMILES: CCc1nc2ccc(Cl)cn2c1C(=O)NCc1ccc(N2CCN(c3ccc(OC(F)(F)F)cc3F)[C@@H](CC)C2)c(F)c1 ; Cl] | A | 3.25 | 622.15 |
| I-2-11 | 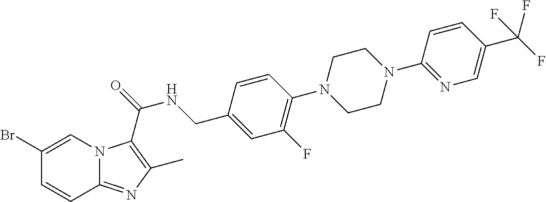 [SMILES: Cc1nc2ccc(Br)cn2c1C(=O)NCc1ccc(N2CCN(c3ccc(C(F)(F)F)cn3)CC2)c(F)c1] | A | 2.6 | 593.1 |
| I-2-12 | 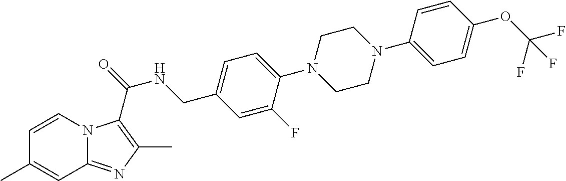 [SMILES: Cc1ccn2c(C(=O)NCc3ccc(N4CCN(c5ccc(OC(F)(F)F)cc5)CC4)c(F)c3)c(C)nc2c1] | B | 2.86 | 542.2 |

TABLE 29

| | | | | |
|---|---|---|---|---|
| I-2-13 | 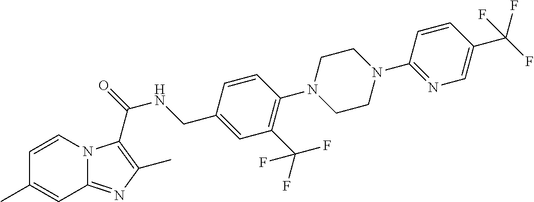 [SMILES: Cc1ccn2c(C(=O)NCc3ccc(N4CCN(c5ccc(C(F)(F)F)cn5)CC4)c(C(F)(F)F)c3)c(C)nc2c1] | B | 2.99 | 577.25 |
| I-2-14 | 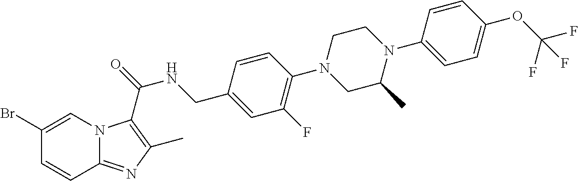 [SMILES: Cc1nc2ccc(Br)cn2c1C(=O)NCc1ccc(N2CCN(c3ccc(OC(F)(F)F)cc3)[C@@H](C)C2)c(F)c1] | A | 2.92 | 622.1 |
| I-2-15 | 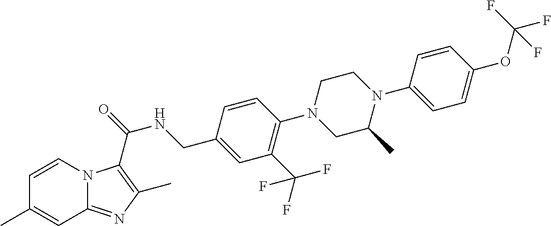 [SMILES: Cc1ccn2c(C(=O)NCc3ccc(N4CCN(c5ccc(OC(F)(F)F)cc5)[C@@H](C)C4)c(C(F)(F)F)c3)c(C)nc2c1] | B | 3.23 | 606.25 |
| I-2-16 | 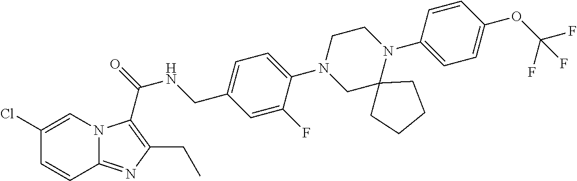 [SMILES: CCc1nc2ccc(Cl)cn2c1C(=O)NCc1ccc(N2CCN(c3ccc(OC(F)(F)F)cc3)C3(CCCC3)C2)c(F)c1] | A | 3.19 | 630.2 |

The following Compounds were also preferable and obtained in accordance with the general synthetic methods and Examples. The chemical structures and the physical properties (LC/MS data) of Compounds are described below.

TABLE 30

| | | LCMS | | MS |
|---|---|---|---|---|
| No. | Structure | method | RT | (M + H) |
| I-3-1 | 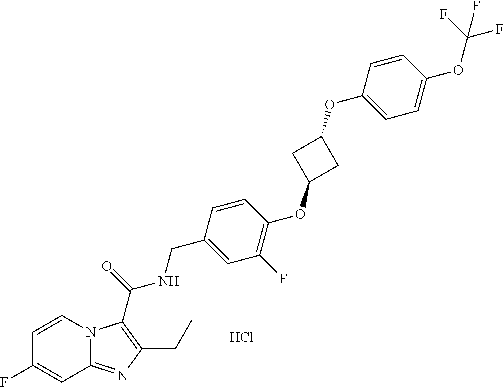 [SMILES: CCc1nc2cc(F)ccn2c1C(=O)NCc1ccc(O[C@H]2C[C@H](Oc3ccc(OC(F)(F)F)cc3)C2)c(F)c1 ; Cl] | A | 2.63 | 562.2 |
| I-3-2 | 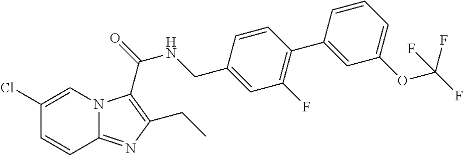 [SMILES: CCc1nc2ccc(Cl)cn2c1C(=O)NCc1ccc(-c2cccc(OC(F)(F)F)c2)c(F)c1] | A | 2.75 | 492.1 |
| I-3-3 | 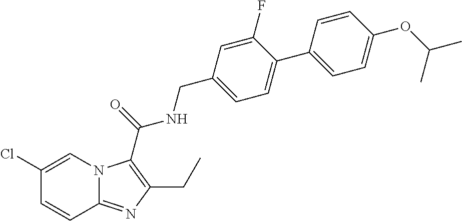 [SMILES: CCc1nc2ccc(Cl)cn2c1C(=O)NCc1ccc(-c2ccc(OC(C)C)cc2)c(F)c1] | A | 2.66 | 466.15 |
| I-3-4 | 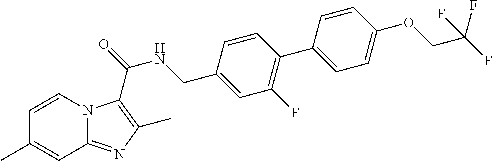 [SMILES: Cc1ccn2c(C(=O)NCc3ccc(-c4ccc(OCC(F)(F)F)cc4)c(F)c3)c(C)nc2c1] | A | 2.13 | 472.15 |
| I-3-5 | 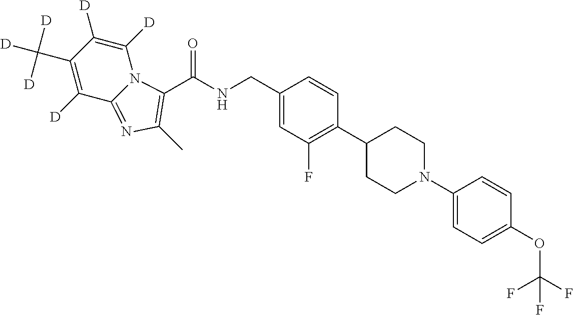 [SMILES: [2H]c1c(C([2H])([2H])[2H])c([2H])c2nc(C)c(C(=O)NCc3ccc(C4CCN(c5ccc(OC(F)(F)F)cc5)CC4)c(F)c3)n2c1[2H]] | B | 3.04 | 547.25 |
| I-3-6 | 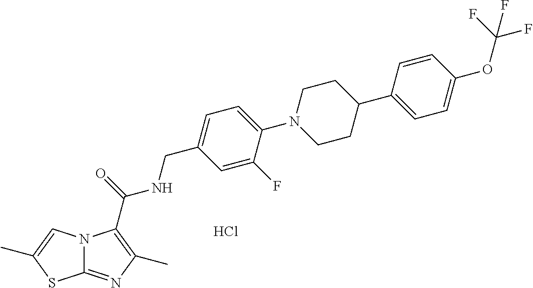 [SMILES: Cc1cn2c(C(=O)NCc3ccc(N4CCC(c5ccc(OC(F)(F)F)cc5)CC4)c(F)c3)c(C)nc2s1 ; Cl] | A | 2.86 | 547.15 |

TABLE 31

| | | | | |
|---|---|---|---|---|
| I-3-7 | 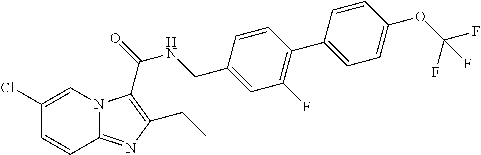 [SMILES: CCc1nc2ccc(Cl)cn2c1C(=O)NCc1ccc(-c2ccc(OC(F)(F)F)cc2)c(F)c1] | A | 2.72 | 492.1 |
| I-3-8 | 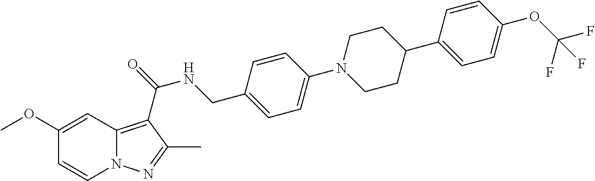 [SMILES: COc1ccn2nc(C)c(C(=O)NCc3ccc(N4CCC(c5ccc(OC(F)(F)F)cc5)CC4)cc3)c2c1] | A | 2.2 | 539.25 |
| I-3-9 | 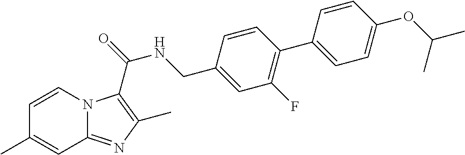 [SMILES: Cc1ccn2c(C(=O)NCc3ccc(-c4ccc(OC(C)C)cc4)c(F)c3)c(C)nc2c1] | B | 2.69 | 432.2 |
| I-3-10 | 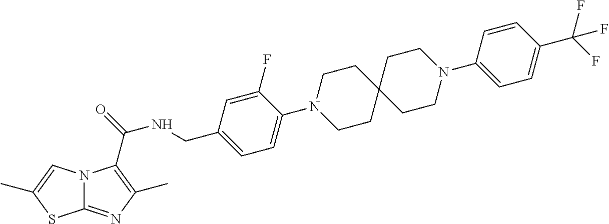 [SMILES: Cc1cn2c(C(=O)NCc3ccc(N4CCC5(CCN(c6ccc(C(F)(F)F)cc6)CC5)CC4)c(F)c3)c(C)nc2s1] | A | 2.98 | 600.25 |
| I-3-11 | 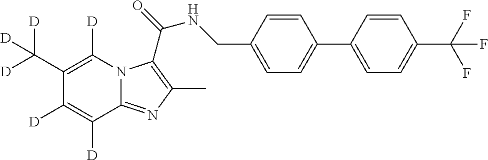 [SMILES: [2H]c1c(C([2H])([2H])[2H])c([2H])n2c(C(=O)NCc3ccc(-c4ccc(C(F)(F)F)cc4)cc3)c(C)nc2c1[2H]] | B | 2.69 | 430.15 |
| I-3-12 | 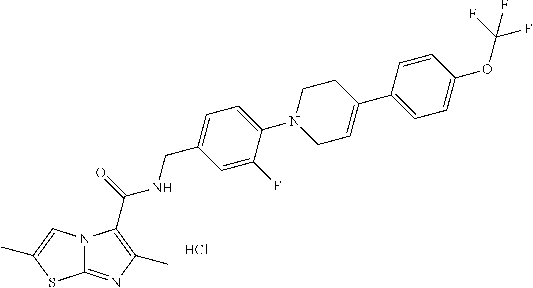 [SMILES: Cc1cn2c(C(=O)NCc3ccc(N4CC=C(c5ccc(OC(F)(F)F)cc5)CC4)c(F)c3)c(C)nc2s1 ; Cl] | A | 2.87 | 545.15 |

TABLE 32

| | | | | |
|---|---|---|---|---|
| I-3-13 | 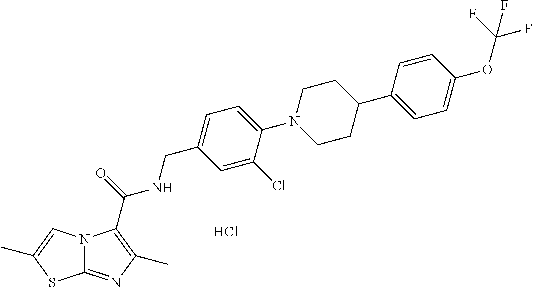 [SMILES: Cc1cn2c(C(=O)NCc3ccc(N4CCC(c5ccc(OC(F)(F)F)cc5)CC4)c(Cl)c3)c(C)nc2s1 ; Cl] | A | 3.08 | 563.1 |
| I-3-14 | 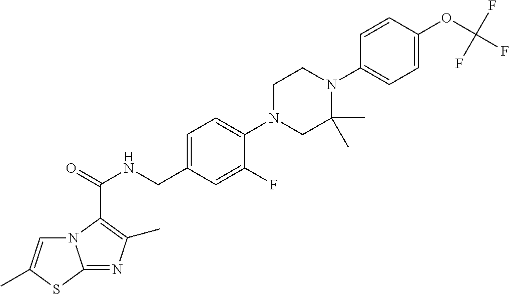 [SMILES: Cc1cn2c(C(=O)NCc3ccc(N4CCN(c5ccc(OC(F)(F)F)cc5)C(C)(C)C4)c(F)c3)c(C)nc2s1] | A | 2.63 | 576.2 |
| I-3-15 | 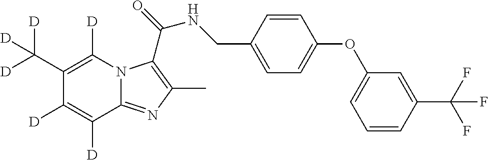 [SMILES: [2H]c1c(C([2H])([2H])[2H])c([2H])n2c(C(=O)NCc3ccc(Oc4cccc(C(F)(F)F)c4)cc3)c(C)nc2c1[2H]] | B | 2.68 | 446.1 |
| I-3-16 | 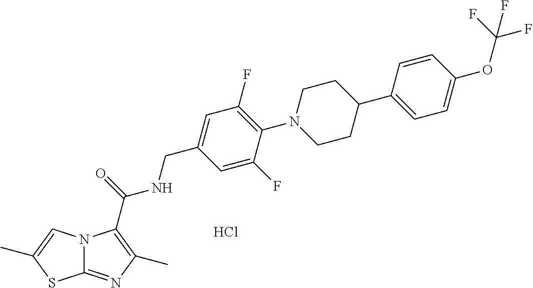 [SMILES: Cc1cn2c(C(=O)NCc3cc(F)c(N4CCC(c5ccc(OC(F)(F)F)cc5)CC4)c(F)c3)c(C)nc2s1 ; Cl] | A | 3.09 | 565.15 |
| I-3-17 | 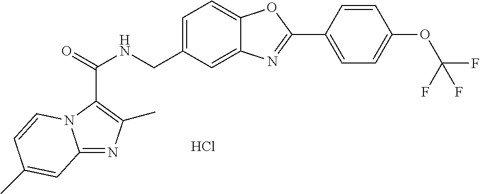 [SMILES: Cc1ccn2c(C(=O)NCc3ccc4oc(-c5ccc(OC(F)(F)F)cc5)nc4c3)c(C)nc2c1 ; Cl] | B | 2.65 | 481.15 |
| I-3-18 | 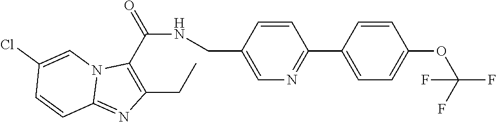 [SMILES: CCc1nc2ccc(Cl)cn2c1C(=O)NCc1ccc(-c2ccc(OC(F)(F)F)cc2)nc1] | A | 2.32 | 475.1 |

TABLE 33

| | | | | |
|---|---|---|---|---|
| I-3-19 | 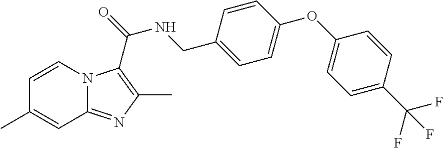 [SMILES: Cc1ccn2c(C(=O)NCc3ccc(Oc4ccc(C(F)(F)F)cc4)cc3)c(C)nc2c1] | B | 2.67 | 440.15 |
| I-3-20 | 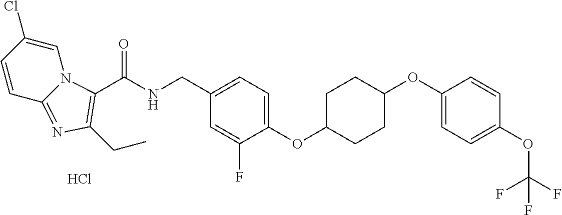 [SMILES: CCc1nc2ccc(Cl)cn2c1C(=O)NCc1ccc(OC2CCC(Oc3ccc(OC(F)(F)F)cc3)CC2)c(F)c1 ; Cl] | A | 3.06 | 606.2 |
| I-3-21 | 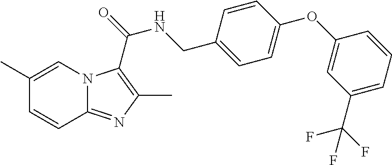 [SMILES: Cc1ccc2nc(C)c(C(=O)NCc3ccc(Oc4cccc(C(F)(F)F)c4)cc3)n2c1] | B | 2.68 | 440.15 |
| I-3-22 | 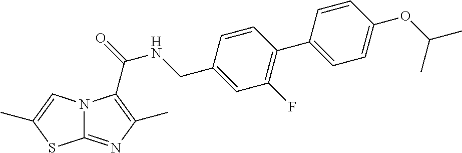 [SMILES: Cc1cn2c(C(=O)NCc3ccc(-c4ccc(OC(C)C)cc4)c(F)c3)c(C)nc2s1] | A | 2.53 | 438.15 |
| I-3-23 | 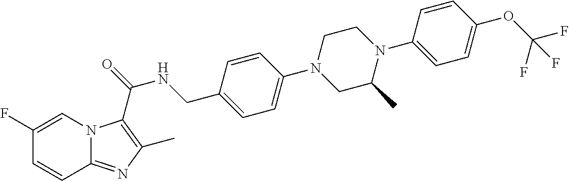 [SMILES: Cc1nc2ccc(F)cn2c1C(=O)NCc1ccc(N2CCN(c3ccc(OC(F)(F)F)cc3)[C@@H](C)C2)cc1] | A | 2.51 | 542.2 |
| I-3-24 | 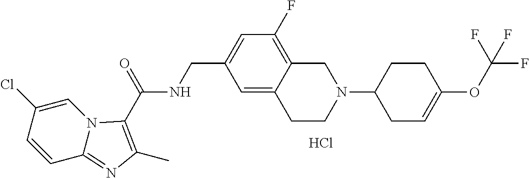 [SMILES: Cc1nc2ccc(Cl)cn2c1C(=O)NCc1cc(F)c2c(c1)CCN(C1CC=C(OC(F)(F)F)CC1)C2 ; Cl] | A | 2.8 | 533.1 |

TABLE 34

| | | | | |
|---|---|---|---|---|
| I-3-25 | 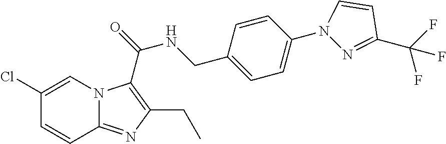 [SMILES: CCc1nc2ccc(Cl)cn2c1C(=O)NCc1ccc(-n2ccc(C(F)(F)F)n2)cc1] | A | 2.26 | 448.1 |
| I-3-26 | 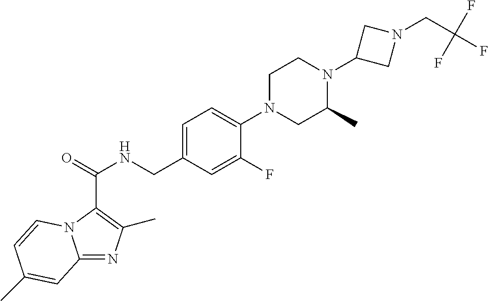 [SMILES: Cc1ccn2c(C(=O)NCc3ccc(N4CCN(C5CN(CC(F)(F)F)C5)[C@@H](C)C4)c(F)c3)c(C)nc2c1] | B | 2.11 | 533.3 |
| I-3-27 | 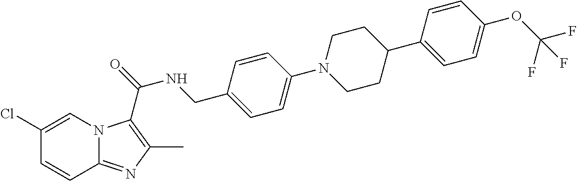 [SMILES: Cc1nc2ccc(Cl)cn2c1C(=O)NCc1ccc(N2CCC(c3ccc(OC(F)(F)F)cc3)CC2)cc1] | A | 2.24 | 543.15 |
| I-3-28 | 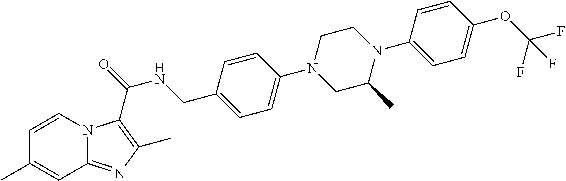 [SMILES: Cc1ccn2c(C(=O)NCc3ccc(N4CCN(c5ccc(OC(F)(F)F)cc5)[C@@H](C)C4)cc3)c(C)nc2c1] | B | 2.92 | 538.25 |
| I-3-29 | 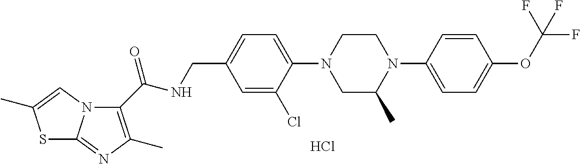 [SMILES: Cc1cn2c(C(=O)NCc3ccc(N4CCN(c5ccc(OC(F)(F)F)cc5)[C@@H](C)C4)c(Cl)c3)c(C)nc2s1 ; Cl] | A | 3 | 578.15 |
| I-3-30 | 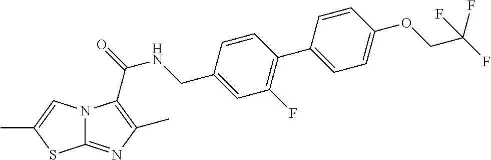 [SMILES: Cc1cn2c(C(=O)NCc3ccc(-c4ccc(OCC(F)(F)F)cc4)c(F)c3)c(C)nc2s1] | A | 2.47 | 478.1 |

TABLE 35

| | | | | |
|---|---|---|---|---|
| I-3-31 | 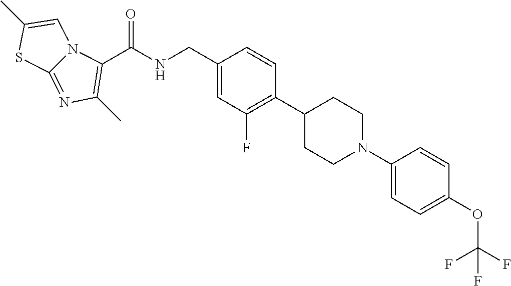 [SMILES: Cc1cn2c(C(=O)NCc3ccc(C4CCN(c5ccc(OC(F)(F)F)cc5)CC4)c(F)c3)c(C)nc2s1] | A | 2.8 | 547.15 |
| I-3-32 | 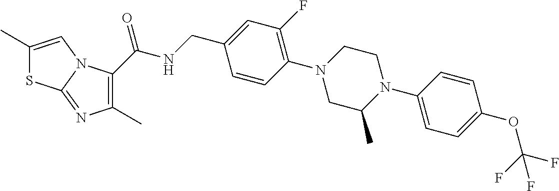 [SMILES: Cc1cn2c(C(=O)NCc3ccc(N4CCN(c5ccc(OC(F)(F)F)cc5)[C@@H](C)C4)c(F)c3)c(C)nc2s1] | A | 2.83 | 562.2 |
| I-3-33 | 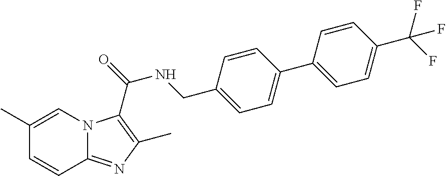 [SMILES: Cc1ccc2nc(C)c(C(=O)NCc3ccc(-c4ccc(C(F)(F)F)cc4)cc3)n2c1] | A | 2.68 | 424.15 |
| I-3-34 | 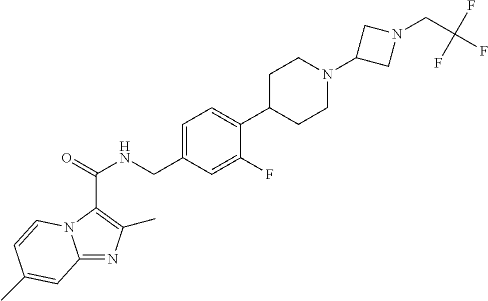 [SMILES: Cc1ccn2c(C(=O)NCc3ccc(C4CCN(C5CN(CC(F)(F)F)C5)CC4)c(F)c3)c(C)nc2c1] | B | 2.22 | 518.3 |
| I-3-35 | 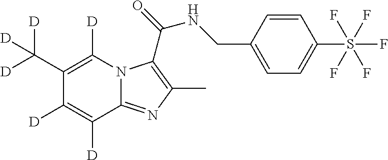 [SMILES: [2H]c1c(C([2H])([2H])[2H])c([2H])n2c(C(=O)NCc3ccc(S(F)(F)(F)(F)F)cc3)c(C)nc2c1[2H]] | B | 2.36 | 412.1 |
| I-3-36 | 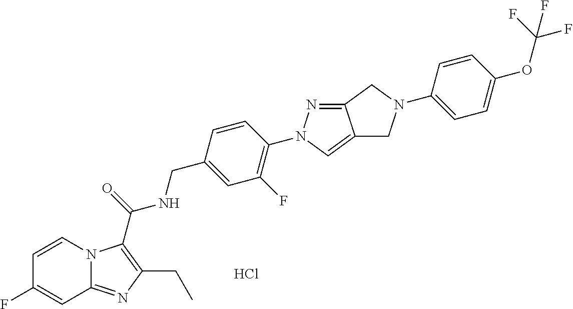 [SMILES: CCc1nc2cc(F)ccn2c1C(=O)NCc1ccc(-n2cc3c(n2)CN(c2ccc(OC(F)(F)F)cc2)C3)c(F)c1 ; Cl] | B | 2.99 | 583.2 |

TABLE 36

| | | | | |
|---|---|---|---|---|
| I-3-37 | 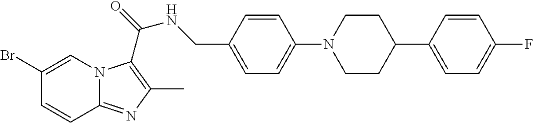 [SMILES: Cc1nc2ccc(Br)cn2c1C(=O)NCc1ccc(N2CCC(c3ccc(F)cc3)CC2)cc1] | A | 1.9 | 521.1 |
| I-3-38 | 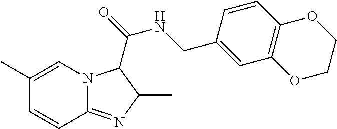 [SMILES: CC1=CN2C(=NC(C)C2C(=O)NCc2ccc3c(c2)OCCO3)C=C1] | B | 1.87 | 338.15 |
| I-3-39 | 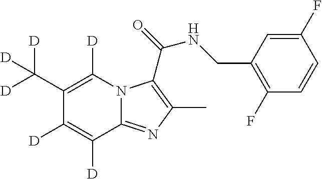 [SMILES: [2H]c1c(C([2H])([2H])[2H])c([2H])n2c(C(=O)NCc3cc(F)ccc3F)c(C)nc2c1[2H]] | A | 1.43 | 322.1 |
| I-3-40 | 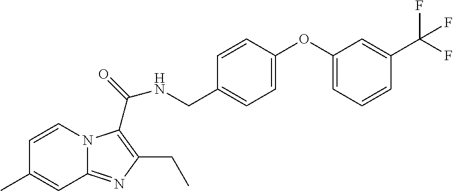 [SMILES: CCc1nc2cc(C)ccn2c1C(=O)NCc1ccc(Oc2cccc(C(F)(F)F)c2)cc1] | B | 2.76 | 454.15 |
| I-3-41 | 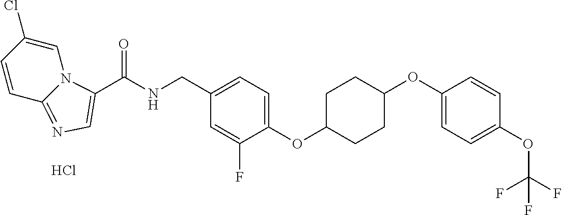 [SMILES: O=C(NCc1ccc(OC2CCC(Oc3ccc(OC(F)(F)F)cc3)CC2)c(F)c1)c1cnc2ccc(Cl)cn12 ; Cl] | A | 3.01 | 606.15 |
| I-3-42 | 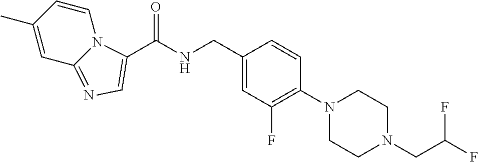 [SMILES: Cc1ccn2c(C(=O)NCc3ccc(N4CCN(CC(F)F)CC4)c(F)c3)cnc2c1] | B | 2.09 | 446.2 |

TABLE 37

| | | | | |
|---|---|---|---|---|
| I-3-43 | 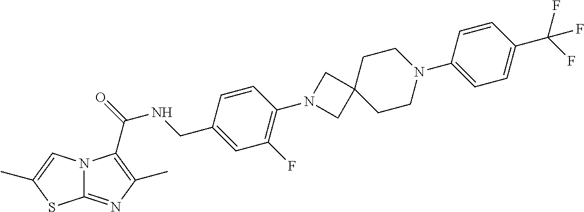 [SMILES: Cc1cn2c(C(=O)NCc3ccc(N4CC5(CCN(c6ccc(C(F)(F)F)cc6)CC5)C4)c(F)c3)c(C)nc2s1] | A | 2.92 | 572.2 |
| I-3-44 | 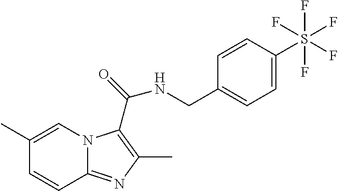 [SMILES: Cc1ccc2nc(C)c(C(=O)NCc3ccc(S(F)(F)(F)(F)F)cc3)n2c1] | B | 2.37 | 406.05 |
| I-3-45 | 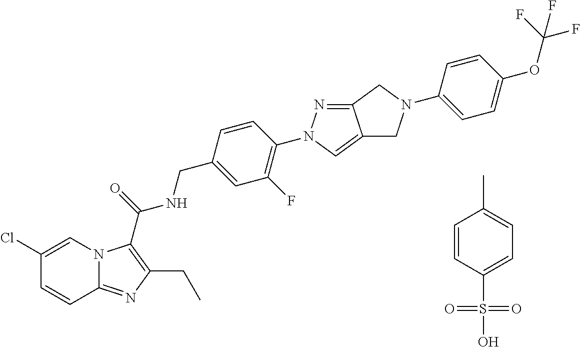 [SMILES: CCc1nc2ccc(Cl)cn2c1C(=O)NCc1ccc(-n2cc3c(n2)CN(c2ccc(OC(F)(F)F)cc2)C3)c(F)c1 ; Cc1ccc(S(=O)(=O)O)cc1] | A | 2.93 | 599.1 |
| I-3-46 | 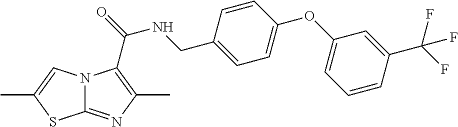 [SMILES: Cc1cn2c(C(=O)NCc3ccc(Oc4cccc(C(F)(F)F)c4)cc3)c(C)nc2s1] | A | 2.5 | 446.1 |
| I-3-47 | 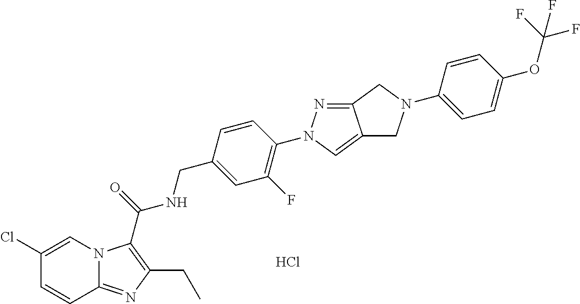 [SMILES: CCc1nc2ccc(Cl)cn2c1C(=O)NCc1ccc(-n2cc3c(n2)CN(c2ccc(OC(F)(F)F)cc2)C3)c(F)c1 ; Cl] | A | 2.93 | 599.1 |
| I-3-48 | 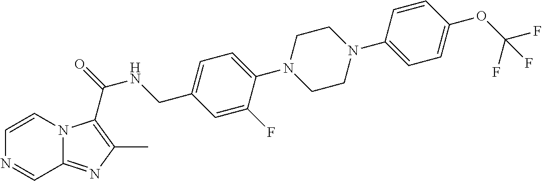 [SMILES: Cc1nc2cnccn2c1C(=O)NCc1ccc(N2CCN(c3ccc(OC(F)(F)F)cc3)CC2)c(F)c1] | A | 2.51 | 529.2 |

TABLE 38

| | | | | |
|---|---|---|---|---|
| I-3-49 | 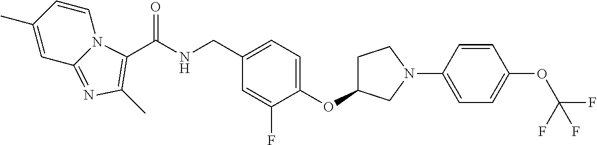 [SMILES: Cc1ccn2c(C(=O)NCc3ccc(O[C@H]4CCN(c5ccc(OC(F)(F)F)cc5)C4)c(F)c3)c(C)nc2c1] | B | 2.88 | 543.2 |
| I-3-50 | 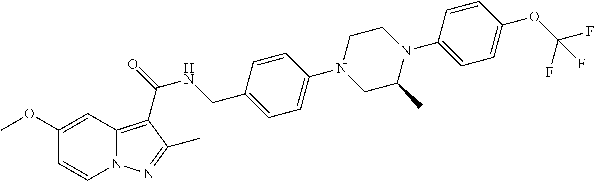 [SMILES: COc1ccn2nc(C)c(C(=O)NCc3ccc(N4CCN(c5ccc(OC(F)(F)F)cc5)[C@@H](C)C4)cc3)c2c1] | A | 2.69 | 554.2 |

### Pharmacological Examples

### Test Example 1

Determination of IC85 for Testing Compounds Against M. avium.

### Preparation

One μL of DMSO stock solutions (200× final concentration) of experimental compounds were added to round-bottom, sterile 96 well microtiter plates. Serial 4-fold dilutions (from 8 to 0.0000076 μM) were made directly in the microtiter plates from column 1 to 11. Untreated control samples with and without inoculum were included in column 12 in each plate.

A sample of Mycobacterium avium ATCC700898 was taken from 7H9 (5% OADC) agar plate. This was first diluted by CAMHB medium to obtain an optical density of 0.1 at 600 nm wavelength and then diluted 1/20, resulting in an inoculum of approximately 5×10 exp6 colony forming units per mL. Microtiter plates were filled with 200 μL of inoculum solution.

Plates were incubated at 37° C. in a stainless-steel bat to prevent evaporation. After 3 days of incubation, resazurin was added to all wells. One day later, fluorescence was measured on EnVision Microplate Reader with 543 excitation and 590 nm emission wavelengths and calculated IC85 values.

The results of Test Example 1 are shown below.

TABLE 39

| | IC85 |
|---|---|
| No. | (uM) |
| I-1-1 | 0.017 |
| I-1-2 | 0.021 |
| I-1-3 | 0.003 |
| I-1-4 | 0.1 |
| I-1-5 | 0.048 |
| I-1-6 | 0.2 |
| I-1-7 | 0.17 |
| I-1-8 | 0.58 |
| I-1-9 | 0.94 |
| I-1-10 | 0.0031 |
| I-1-11 | 0.089 |
| I-1-12 | 0.07 |
| I-1-13 | 0.057 |
| I-1-14 | 0.76 |
| I-1-15 | 0.057 |
| I-1-16 | 0.0016 |
| I-1-17 | 0.013 |
| I-1-18 | 0.011 |
| I-1-19 | 0.13 |
| I-1-20 | 0.033 |
| I-1-21 | 0.038 |
| I-1-22 | 0.028 |
| I-1-23 | 0.045 |
| I-1-24 | 0.032 |
| I-1-25 | 0.0022 |
| I-1-26 | 0.015 |
| I-1-27 | 0.033 |
| I-1-28 | 0.012 |
| I-1-29 | 0.0027 |
| I-1-30 | 0.0031 |
| I-1-31 | 0.14 |
| I-1-32 | 0.0091 |
| I-1-33 | 0.068 |
| I-1-34 | 0.008 |
| I-1-35 | 0.026 |
| I-1-36 | 0.11 |
| I-1-37 | 0.036 |
| I-1-38 | 0.0013 |
| I-1-39 | 0.011 |
| I-1-40 | 0.022 |
| I-1-41 | 0.18 |
| I-1-42 | 0.009 |
| I-1-43 | 0.015 |
| I-1-44 | 0.011 |
| I-1-45 | 0.011 |
| I-1-46 | 0.0026 |
| I-1-47 | 0.11 |
| I-1-48 | 0.035 |
| I-1-49 | 0.068 |
| I-1-50 | 0.5 |
| I-1-51 | 0.11 |
| I-1-52 | 0.0054 |
| I-1-53 | 0.037 |
| I-1-54 | 0.034 |
| I-1-55 | 0.69 |
| I-1-56 | 0.022 |
| I-1-57 | 0.0036 |
| I-1-58 | 0.077 |
| I-1-59 | 0.011 |
| I-1-60 | 0.011 |
| I-1-61 | 0.2 |
| I-1-62 | 0.0072 |
| I-1-63 | 0.012 |
| I-1-64 | 0.079 |
| I-1-65 | 0.12 |
| I-1-66 | 0.011 |
| I-1-67 | 0.068 |
| I-1-68 | 0.013 |
| I-1-69 | 0.51 |
| I-1-70 | 0.025 |
| I-1-71 | 0.1 |
| I-1-72 | 0.066 |
| I-1-73 | 0.019 |
| I-1-74 | 0.039 |
| I-1-75 | 0.016 |
| I-1-76 | 0.046 |
| I-1-77 | 0.022 |
| I-1-78 | 0.037 |
| I-1-79 | 0.036 |
| I-1-80 | 0.06 |
| I-1-81 | 0.33 |
| I-1-82 | 0.5 |
| I-1-83 | 0.62 |
| I-1-84 | 0.038 |
| I-1-85 | 0.0069 |
| I-1-86 | 0.028 |
| I-1-87 | 0.0079 |
| I-1-88 | 0.0061 |
| I-1-89 | 0.0072 |
| I-1-90 | 0.0052 |
| I-1-91 | 0.0053 |
| I-1-92 | 0.0055 |
| I-1-93 | 0.011 |
| I-1-94 | 0.014 |
| I-1-95 | 0.0022 |
| I-1-96 | 0.011 |
| I-1-97 | 0.0072 |
| I-1-98 | 0.0088 |
| I-1-99 | 0.035 |
| I-1-100 | 0.046 |
| I-1-101 | 0.47 |
| I-1-102 | 0.024 |
| I-1-103 | 0.019 |
| I-1-104 | 0.092 |
| I-1-105 | 0.059 |
| I-1-106 | 0.0062 |
| I-1-107 | 0.007 |
| I-1-108 | 0.0042 |
| I-1-109 | 0.0071 |
| I-1-110 | 0.0093 |
| I-1-111 | 0.0071 |
| I-1-112 | 0.0014 |
| I-1-113 | 0.0048 |
| I-1-114 | 0.0074 |
| I-1-115 | 0.0046 |
| I-1-116 | 0.011 |
| I-1-117 | 0.0025 |
| I-1-118 | 0.0076 |
| I-1-119 | 0.0065 |
| I-1-120 | 0.012 |
| I-1-121 | 0.003 |
| I-1-122 | 0.066 |
| I-1-123 | 0.014 |
| I-1-124 | 0.87 |
| I-1-125 | 0.78 |
| I-1-126 | 0.27 |
| I-1-127 | 0.0077 |
| I-1-128 | 0.018 |
| I-1-129 | 0.0068 |
| I-1-130 | 0.049 |
| I-1-131 | 0.045 |
| I-1-132 | 0.014 |
| I-1-133 | 0.019 |
| I-1-134 | 0.043 |
| I-1-135 | 0.12 |
| I-1-136 | 0.016 |
| I-1-137 | 0.1 |
| I-1-138 | 0.0068 |
| I-1-139 | 0.009 |
| I-1-140 | 0.0057 |
| I-1-141 | 0.002 |
| I-1-142 | 0.03 |
| I-1-143 | 0.023 |
| I-1-144 | 0.017 |
| I-1-145 | 0.016 |
| I-1-146 | 0.026 |
| I-1-147 | 0.0083 |
| I-1-148 | 0.0054 |
| I-1-149 | 0.015 |
| I-1-150 | 0.0024 |
| I-1-151 | 0.01 |
| I-1-152 | 0.11 |
| I-1-153 | 0.031 |

TABLE 40

| | IC85 |
|---|---|
| No. | (uM) |
| I-2-1 | 0.016 |
| I-2-2 | 0.37 |
| I-2-3 | 0.017 |
| I-2-4 | 0.0065 |
| I-2-5 | 0.42 |
| I-2-6 | 0.0044 |
| I-2-7 | 0.59 |
| I-2-8 | 0.13 |
| I-2-9 | 0.019 |
| I-2-10 | 0.023 |
| I-2-11 | 0.02 |
| I-2-12 | 0.023 |
| I-2-13 | 0.35 |
| I-2-14 | 0.0045 |
| I-2-15 | 0.25 |
| I-2-16 | 0.086 |

TABLE 41

| | IC85 |
|---|---|
| No. | (uM) |
| I-3-1 | 0.054 |
| I-3-2 | 0.0023 |
| I-3-3 | 0.0094 |
| I-3-4 | 0.043 |
| I-3-5 | 0.021 |
| I-3-6 | 0.26 |
| I-3-7 | 0.056 |
| I-3-8 | 0.028 |
| I-3-9 | 0.13 |
| I-3-10 | 0.029 |
| I-3-11 | 0.0028 |
| I-3-12 | 0.34 |
| I-3-13 | 0.35 |
| I-3-14 | 0.19 |
| I-3-15 | 0.23 |
| I-3-16 | 0.11 |
| I-3-17 | 0.86 |
| I-3-18 | 0.19 |
| I-3-19 | 0.44 |
| I-3-20 | 0.018 |
| I-3-21 | 0.13 |
| I-3-22 | 0.37 |
| I-3-23 | 0.015 |
| I-3-24 | 0.13 |
| I-3-25 | 0.14 |
| I-3-26 | 0.96 |
| I-3-27 | 0.012 |
| I-3-28 | 0.036 |
| I-3-29 | 0.061 |
| I-3-30 | 0.031 |
| I-3-31 | 0.33 |
| I-3-32 | 0.82 |
| I-3-33 | 0.0022 |
| I-3-34 | 0.61 |
| I-3-35 | 0.11 |
| I-3-36 | 0.2 |
| I-3-37 | 0.016 |
| I-3-38 | 0.86 |
| I-3-39 | 0.8 |
| I-3-40 | 0.59 |
| I-3-41 | 0.15 |
| I-3-42 | 0.93 |
| I-3-43 | 0.15 |
| I-3-44 | 0.11 |
| I-3-45 | 0.0046 |
| I-3-46 | 0.61 |
| I-3-47 | 0.0051 |
| I-3-48 | 0.66 |
| I-3-49 | 0.5 |
| I-3-50 | 0.0058 |

### Test Example 2

### Metabolic Stability Test

Using a commercially available pooled human liver microsomes, a compound of the present invention was reacted for a constant time, a remaining rate was calculated by comparing a reacted sample and an unreacted sample, thereby, a degree of metabolism in liver was assessed.

A reaction was performed (oxidative reaction) at 37° C. for 0 minute or 30 minutes in the presence of 1 mmol/L NADPH in 0.2 mL of a buffer (50 mmol/L Tris-HCl pH 7.4, 150 mmol/L potassium chloride, 10 mmol/L magnesium chloride) containing 0.5 mg protein/mL of human liver microsomes. After the reaction, 50 μL of the reaction solution was added to 100 μL of a methanol/acetonitrile=1/1 (v/v), mixed and centrifuged at 3000 rpm for 15 minutes. The compound of the present invention in the supernatant was quantified by LC/MS/MS or solid phase extraction (SPE)/MS, and a remaining amount of the compound of the present invention after the reaction was calculated, letting a compound amount at 0 minute reaction time to be 100%.

The results of Test Example 2 are shown below.

TABLE 42

| No. | MS_h_% |
|---|---|
| I-1-1 | 75 |
| I-1-2 | 86 |
| I-1-3 | 66 |
| I-1-4 | 88 |
| I-1-5 | 70 |
| I-1-6 | 60 |
| I-1-7 | 87 |
| I-1-8 | 86 |
| I-1-9 | 82 |
| I-1-10 | 60 |
| I-1-11 | 72 |
| I-1-12 | 64 |
| I-1-13 | 82 |
| I-1-14 | 79 |
| I-1-15 | 71 |
| I-1-16 | 67 |
| I-1-17 | 63 |
| I-1-18 | 85 |
| I-1-19 | 87 |
| I-1-20 | 83 |
| I-1-21 | 89 |
| I-1-22 | 87 |
| I-1-23 | 85 |
| I-1-24 | 62 |
| I-1-25 | 72 |
| I-1-26 | 93 |
| I-1-27 | 80 |
| I-1-28 | 94 |
| I-1-29 | 89 |
| I-1-30 | 89 |
| I-1-31 | 82 |
| I-1-32 | 62 |
| I-1-33 | 85 |
| I-1-34 | 65 |
| I-1-35 | 91 |
| I-1-36 | 79 |
| I-1-37 | 76 |
| I-1-38 | 90 |
| I-1-39 | 75 |
| I-1-40 | 89 |
| I-1-41 | 86 |
| I-1-42 | 59 |
| I-1-43 | 75 |
| I-1-44 | 69 |
| I-1-45 | 64 |
| I-1-46 | 96 |
| I-1-47 | 85 |
| I-1-48 | 69 |
| I-1-49 | 59 |
| I-1-50 | 94 |
| I-1-51 | 90 |
| I-1-52 | 84 |
| I-1-53 | 65 |
| I-1-54 | 79 |
| I-1-55 | 84 |
| I-1-56 | 60 |
| I-1-57 | 84 |
| I-1-58 | 89 |
| I-1-59 | 87 |
| I-1-60 | 72 |
| I-1-61 | 95 |
| I-1-62 | 82 |
| I-1-63 | 95 |
| I-1-64 | 94 |
| I-1-65 | 96 |
| I-1-66 | 83 |
| I-1-67 | 93 |
| I-1-68 | 91 |
| I-1-69 | 89 |
| I-1-70 | 84 |
| I-1-71 | 78 |
| I-1-72 | 87 |
| I-1-73 | 90 |
| I-1-74 | 64 |
| I-1-75 | 71 |
| I-1-76 | 80 |
| I-1-77 | 59 |
| I-1-78 | 69 |
| I-1-79 | 65 |
| I-1-80 | 74 |
| I-1-81 | 94 |
| I-1-82 | 98 |
| I-1-83 | 85 |
| I-1-84 | 86 |
| I-1-85 | 91 |
| I-1-86 | 77 |
| I-1-87 | 86 |
| I-1-88 | 89 |
| I-1-89 | 65 |
| I-1-90 | 91 |
| I-1-91 | 89 |
| I-1-92 | 66 |
| I-1-93 | 67 |
| I-1-94 | 67 |
| I-1-95 | 91 |
| I-1-96 | 90 |
| I-1-97 | 86 |
| I-1-98 | 76 |
| I-1-99 | 77 |
| I-1-100 | 65 |
| I-1-101 | 90 |
| I-1-102 | 75 |
| I-1-103 | 64 |
| I-1-104 | 72 |
| I-1-105 | 77 |
| I-1-106 | 59 |
| I-1-107 | 71 |
| I-1-108 | 71 |
| I-1-109 | 80 |
| I-1-110 | 80 |
| I-1-111 | 88 |
| I-1-112 | 69 |
| I-1-113 | 79 |
| I-1-114 | 87 |
| I-1-115 | 69 |
| I-1-116 | 87 |
| I-1-117 | 83 |
| I-1-118 | 97 |
| I-1-119 | 63 |
| I-1-120 | 71 |
| I-1-121 | 95 |
| I-1-122 | 81 |
| I-1-123 | 77 |
| I-1-124 | 63 |
| I-1-125 | 68 |
| I-1-126 | 77 |
| I-1-127 | 93 |
| I-1-128 | 85 |
| I-1-129 | 80 |
| I-1-130 | 74 |
| I-1-131 | 73 |
| I-1-132 | 62 |
| I-1-133 | 81 |
| I-1-134 | 65 |
| I-1-135 | 75 |
| I-1-136 | 86 |
| I-1-137 | 91 |
| I-1-138 | 83 |
| I-1-139 | 61 |
| I-1-140 | 89 |
| I-1-141 | 93 |
| I-1-142 | 71 |
| I-1-143 | 77 |
| I-1-144 | 67 |
| I-1-145 | 80 |
| I-1-146 | 85 |
| I-1-147 | 91 |
| I-1-148 | 73 |
| I-1-149 | 63 |
| I-1-150 | 65 |
| I-1-151 | 88 |
| I-1-152 | 87 |
| I-1-153 | 72 |

TABLE 43

| No. | MS_h_% |
|---|---|
| I-2-1 | 106 |
| I-2-2 | 97.7 |
| I-2-3 | 90.3 |
| I-2-4 | 90.1 |
| I-2-5 | 81.8 |
| I-2-6 | 80.8 |
| I-2-7 | 79.3 |
| I-2-8 | 74.9 |
| I-2-9 | 73.5 |
| I-2-10 | 72.2 |
| I-2-11 | 71.7 |
| I-2-12 | 71.4 |
| I-2-13 | 67.6 |
| I-2-14 | 67 |
| I-2-15 | 59.2 |
| I-2-16 | 58.2 |

TABLE 44

| No. | MS_h_% |
|---|---|
| I-3-1 | 99.1 |
| I-3-2 | 97.7 |
| I-3-3 | 96.1 |
| I-3-4 | 94.6 |
| I-3-5 | 93.7 |
| I-3-6 | 91.7 |
| I-3-7 | 89.5 |
| I-3-8 | 87.1 |
| I-3-9 | 85.9 |
| I-3-10 | 84.9 |
| I-3-11 | 84.7 |
| I-3-12 | 84.4 |
| I-3-13 | 84 |
| I-3-14 | 82.7 |
| I-3-15 | 82.4 |
| I-3-16 | 81.4 |
| I-3-17 | 81.1 |
| I-3-18 | 80.9 |
| I-3-19 | 80.9 |
| I-3-20 | 80.8 |
| I-3-21 | 79.1 |
| I-3-22 | 78.5 |
| I-3-23 | 77.8 |
| I-3-24 | 77.5 |
| I-3-25 | 77 |
| I-3-26 | 77 |
| I-3-27 | 75.5 |
| I-3-28 | 75.4 |
| I-3-29 | 75.1 |
| I-3-30 | 75.1 |
| I-3-31 | 73.9 |
| I-3-32 | 72.6 |
| I-3-33 | 72.6 |
| I-3-34 | 71.9 |
| I-3-35 | 71.3 |
| I-3-36 | 69 |
| I-3-37 | 68 |
| I-3-38 | 67.7 |
| I-3-39 | 67.2 |
| I-3-40 | 66 |
| I-3-41 | 65.4 |
| I-3-42 | 64.6 |
| I-3-43 | 64.1 |
| I-3-44 | 63.9 |
| I-3-45 | 63.2 |
| I-3-46 | 63.1 |
| I-3-47 | 62 |
| I-3-48 | 60.4 |
| I-3-49 | 60.2 |
| I-3-50 | 58.5 |

Usefulness as a medicament can be examined by the following tests, etc.

### Test Example 3: CYP Inhibition Test

Using commercially available pooled human liver microsomes, an inhibitory degree of each metabolite production amount by the compound of the present invention was assessed as marker reactions of human main five CYP isoforms (CYP1A2, 2C9, 2C19, 2D6, and 3A4), 7-ethoxyresorufin O-deethylation (CYP1A2), tolbutamide methyl-hydroxylation (CYP2C9), mephenytoin 4′-hydroxylation (CYP2C19), dextromethorphan O-demethylation (CYP2DC), and terfenedine hydroxylation (CYP3A4).

The reaction conditions were as follows: substrate, 0.5 μmol/L ethoxyresorufin (CYP1A2), 100 μmol/L tolbutamide (CYP2C9), 50 μmol/L S-mephenytoin (CYP2C19), 5 μmol/L dextromethorphan (CYP2D6), 1 μmol/L terfenedine (CYP3A4); reaction time, 15 minutes; reaction temperature, 37° C.; enzyme, pooled human liver microsomes 0.2 mg protein/mL; concentration of the compound of the present invention, 1.0, 5.0, 10, 20 μmol/L (four points).

Each of five kinds of substrates, human liver microsomes, or the compound of the present invention in 50 mmol/L Hepes buffer were added to a 96-well plate at the composition as described above, and NADPH, as a cofactor was added to initiate metabolism reactions. After the incubation at 37° C. for 15 minutes, a methanol/acetonitrile=1/1 (V/V) solution was added to stop the reaction. After the centrifugation at 3000 rpm for 15 minutes, resorufin (CYP1A2 metabolite) in the supernatant was quantified by a fluorescent multilabel counter or LC/MS/MS and hydroxytolbutamide (CYP2C9 metabolite), 4′ hydroxymephenytoin (CYP2C19 metabolite), dextrorphan (CYP2D6 metabolite), and terfenadine alcohol metabolite (CYP3A4 metabolite) were quantified by LC/MS/MS.

The sample obtained by adding only DMSO that is a solvent dissolving a compound instead of the compound of the present invention to a reaction mixture was adopted as a control (100%). Remaining activity (%) was calculated at each concentration of the compound of the present invention compared to control, and IC₅₀ was calculated by reverse presumption by a logistic model using a concentration and an inhibition rate.

### Test Example 4: CYP3A4 (MDZ) MBI Test

The CYP3A4 (MDZ) MBI test is a test of investigating Mechanism based inhibition (MBI) potential on CYP3A4 inhibition of the compound of the present invention by the enhancement of the inhibitory effect caused by a metabolic reaction of the compound of the present invention. CYP3A4 inhibition was evaluated using pooled human liver microsomes by 1-hydroxylation reaction of midazolam (MDZ) as a marker reaction.

The reaction conditions were as follows: substrate, 10 μmol/L MDZ; pre-reaction time, 0 or 30 minutes; substrate metabolic reaction time, 2 minutes; reaction temperature, 37° C.; protein content of pooled human liver microsomes, at pre-reaction 0.5 mg/mL, at reaction 0.05 mg/mL (at 10-fold dilution); concentrations of the compound of the present invention, 1, 5, 10, 20 μmol/L or 0.83, 5, 10, and 20 μmol/L (four points).

Pooled human liver microsomes and a solution of the compound of the present invention in K-Pi buffer (pH 7.4) as a pre-reaction solution were added to a 96-well plate at the composition of the pre-reaction. A part of pre-reaction solution was transferred to another 96-well plate, and 1/10 diluted by K-Pi buffer containing a substrate. NADPH as a cofactor was added to initiate a reaction as a marker reaction (Preincubation 0 min). After a predetermined time of a marker reaction, a solution of methanol/acetonitrile=1/1 (V/V) was added to stop the reaction. In addition, NADPH was added to a remaining pre-reaction solution to initiate a pre-reaction (Preincubation 30 min). After a predetermined time of a pre-reaction, a part was transferred to another 96-well plate, and 1/10 diluted by K-Pi buffer containing a substrate to initiate a reaction as a marker reaction. After a predetermined time of a marker reaction, a solution of methanol/acetonitrile=1/1 (V/V) was added to stop the reaction. After centrifuged at 3000 rpm for 15 minutes, 1-hydroxymidazolam in the supernatant was quantified by LC/MS/MS.

The sample obtained by adding only DMSO that was a solvent dissolving a compound instead of the compound of the present invention to a reaction mixture was adopted as a control (100%). Remaining activity (%) was calculated at each concentration of the compound of the present invention compared to control, and IC value was calculated by reverse-presumption by a logistic model using a concentration and an inhibition rate. Shifted IC value was calculated as “IC of preincubation 0 min/IC of preincubation 30 min”. When a shifted IC was 1.5 or more, this was defined as positive. When a shifted IC was 1.0 or less, this was defined as negative.

### Test Example 5: BA Test

Materials and Methods for experiments to evaluate oral absorption

(1) Animals: rats were used
(2) Breeding conditions: The mice or rats were allowed to freely take solid food and sterilized tap water.
(3) Dose and grouping: orally or intravenously administered at a predetermined dose; grouping was as follows (Dose depends on the compound) Oral administration: 2 to 60 μmol/kg or 1 to 30 mg/kg (n=2 to 3) Intravenous administration: 1 to 30 μmol/kg or 0.5 to 10 mg/kg (n=2 to 3)
(4) Preparation of dosing solution: for oral administration, in a solution or a suspension state; for intravenous administration, in a solubilized state
(5) Administration method: in oral administration, forcedly administered into ventriculus with oral probe; in intravenous administration, administer from caudal vein with a needle-equipped syringe
(6) Evaluation items: blood was collected over time, and the plasma concentration of drug was measured by LC/MS/MS
(7) Statistical analysis: regarding the transition of the plasma concentration of the compound of the present invention, the area under the plasma concentration-time curve (AUC) was calculated by non-linear least squares program WinNonlin (Registered trade name), and the bioavailability (BA) was calculated from the AUCs of the oral administration group and intravenous administration group.

### Test Example 6: Fluctuation Ames Test

Mutagenicity of the compound of the present invention was evaluated.

A 20 μL of freezing-stored Salmonella typhimurium (TA98 strain, TA100 strain) was inoculated on 10 mL of a liquid nutrient medium (2.5% Oxoid nutrient broth No. 2), and this was incubated at 37° C. for 10 hours under shaking. The 7.70 to 8.00 mL of TA98 culture medium was centrifuged (2000×g, 10 minutes). Bacteria were suspended in a Micro F buffer (K₂HPO₄: 3.5 g/L, KH₂PO₄: 1 g/L, (NH₄)₂SO₄: 1 g/L, trisodium citrate dihydrate: 0.25 g/L, and MgSO₄.7H₂0: 0.1 g/L) with the same volume as that of the culture medium used for centrifugation. The suspension was added to 120 mL of Exposure medium (Micro F buffer containing biotin: 8 μg/mL, histidine: 0.2 μg/mL, and glucose: 8 mg/mL). The 3.10 to 3.42 mL of TA100 culture medium strain was mixed with 120 to 130 mL Exposure medium. Each 12 μL of DMSO solution of the compound of the present invention (several stage dilution from maximum dose 50 mg/mL at 2 to 3 fold ratio), DMSO as a negative control, and 50 μg/mL of 4-nitroquinoline 1-oxide DMSO solution for the TA98 strain and 0.25 μg/mL of 2-(2-furyl)-3-(5-nitro-2-furyl)acrylamide DMSO solution for the TA100 strain in the assay without metabolic activation, 40 μg/mL of 2-aminoanthracene DMSO solution for the TA98 strain and 20 μg/mL of 2-aminoanthracene DMSO solution for the TA100 strain in the assay with metabolic activation as a positive control, and 588 μL of the test bacterial suspension (498 μL and 90 μL of S9 mixture in the case of metabolic activation assay) was mixed, and this was incubated at 37° C. for 90 minutes under shaking. 460 μL of the mixture was mixed with 2300 μL of Indicator medium (Micro F buffer containing 8 μg/mL biotin, 0.2 μg/mL histidine, 8 mg/mL glucose, 37.5 μg/mL bromocresol purple), each 50 μL was dispensed to microplate 48 wells/dose, and this was incubated at 37° C. for 3 days. Since the wells containing the bacteria which gained growth ability by point mutation in amino acid (histidine) synthesizing enzyme gene turns from purple to yellow due to a pH change, the number of yellow wells in 48 wells was counted per dose, and was compared with the negative control group. (−) and (+) means negative and positive in mutagenicity respectively.

### Test Example 7: hERG Test

For the purpose of assessing risk of an electrocardiogram QT interval prolongation of the compound of the present invention, effects of the compound of the present invention on delayed rectifier K+ current (I_{Kr}), which plays an important role in the ventricular repolarization process, was studied using CHO cells expressing human ether-a-go-go related gene (hERG) channel.

After a cell was retained at a membrane potential of −80 mV by whole cell patch clamp method using an automated patch clamp system (QPatch; Sophion Bioscience A/S) and gave a leak potential of −50 mV, I_{Kr} induced by depolarization pulse stimulation at +20 mV for 2 seconds and, further, repolarization pulse stimulation at −50 mV for 2 seconds, was recorded. Extracellular solution (NaCl: 145 mmol/L, KCl: 4 mmol/L, CaCl₂): 2 mmol/L, MgCl₂: 1 mmol/L, glucose: 10 mmol/L, HEPES (4-(2-hydroxyethyl)-1-piperazineethanesulfonic acid, 4-(2-hydroxyethyl)-1-piperazineethanesulfonic acid): 10 mmol/L, pH=7.4) adjusted to contain 0.1% dimethylsulfoxide was used as a medium. The extracellular solution in which the medium and the compound of the present invention had been dissolved at each objective concentration was applied to the cell for 7 minutes or more at room temperature. From the recording I_{Kr}, an absolute value of the tail peak current was measured based on the current value at the resting membrane potential using analysis software (QPatch Assay software; Sophion Bioscience A/S). Further, the tail peak current after application of the compound of the present invention relative to the tail peak current after application of the medium was calculated as a % inhibition to assess the influence of the compound of the present invention on I_{Kr}.

### Test Example 8: Solubility Test

The solubility of the compound of the present invention was determined under 1% DMSO addition conditions. 10 mmol/L solution of the compound was prepared with DMSO. 2 μL of the solution of the compound of the present invention was respectively added to 198 μL of JP-1 fluid or JP-2 fluid. The mixture was left shaking at room temperature for 1 hour, and the mixture was vacuum-filtered. The filtrate was 10- or 100-fold diluted with methanol/water=1/1 (v/v) or acetonitrile/methanol/water=1/1/2 (v/v/v), and the compound concentration in the filtrate was measured with LC/MS or Solid-Phase Extraction (SPE)/MS by the absolute calibration method.

The composition of the JP-1 fluid was as below.

Water was added to 2.0 g of sodium chloride and 7.0 mL of hydrochloric acid to reach 1000 mL.

The composition of the JP-2 fluid was as below.

1 volume of water was added to 1 volume of the solution in which 3.40 g of potassium dihydrogen phosphate and 3.55 g of anhydrous disodium hydrogen phosphate were dissolved in water to reach 1000 mL.

### Test Example 9: Powder Solubility Test

Appropriate quantity of the compound of the present invention was put in suitable containers. 200 μL of JP-1 fluid (water was added to 2.0 g of sodium chloride and 7.0 mL of hydrochloric acid to reach 1000 mL), 200 μL of JP-2 fluid (1 volume of water was added to 1 volume of the solution which 3.40 g of potassium dihydrogen phosphate and 3.55 g of anhydrous disodium hydrogen phosphate dissolve in water to reach 1000 mL) or 20 mmol/L sodium taurocholate (TCA)/JP-2 fluid (JP-2 fluid was added to 1.08 g of TCA to reach 100 mL) was independently added to each container. When total amount was dissolved after adding the test reagent, the compound of the present invention was added appropriately. After sealing and shaking at 37° C. for 1 hour, the solution was filtered and 100 μL of methanol was added to 100 μL of each filtrate to dilute two-fold. The dilution rate was changed as necessary. After checking that there is no bubble and precipitate, the container was sealed and shaken. The compound of the present invention was measured using HPLC by absolute calibration curve method.

### Test Example 10: P-g Substrate Test

The compound of the present invention is added to one side of Transwell (registered trademark, CORNING) where human MDR1-expressing cells or parent cells have been monolayer-cultured. The cells are reacted for a constant time. The membrane permeability coefficients from the apical side toward the basolateral side (A→B) and from the basolateral side toward the apical side (B→A) are calculated for the MDR1-expressing cells or the parent cells, and the efflux ratio (ER; ratio of the membrane permeability coefficients of B→A and A→B) values of the MDR1-expressing cells and the parent cells are calculated. The efflux ratio (ER) values of the MDR1-expressing cells and the parent cells are compared to confirm whether or not the compound of the present invention would be a P-gp substrate.

### Formulation Example

The following Formulation Examples are only exemplified and not intended to limit the scope of the invention.

### Formulation Example 1: Tablets

The compounds used in the present invention, lactose, and calcium stearate were mixed. The mixture was crushed, granulated and dried to give a suitable size of granules. Next, calcium stearate was added to the granules, and the mixture was compressed and molded to give tablets.

### Formulation Example 2: Capsules

The compounds used in the present invention, lactose, and calcium stearate were mixed uniformly to obtain powder medicines in the form of powders or fine granules. The powder medicines were filled into capsule containers to give capsules.

### Formulation Example 3: Granules

The compounds used in the present invention, lactose and calcium stearate are mixed uniformly and the mixture is compressed and molded. Then, it is crushed, granulated and sieved to give suitable sizes of granules.

### Formulation Example 4: Orally Disintegrated Tablets

The compounds used in the present invention and crystalline cellulose are mixed, granulated and tablets are made to give orally disintegrated tablets.

### Formulation Example 5: Dry Syrups

The compounds used in the present invention and lactose are mixed, crushed, granulated and sieved to give suitable sizes of dry syrups.

### Formulation Example 6: Injections

The compounds used in the present invention and phosphate buffer are mixed to give injection.

### Formulation Example 7: Infusions

The compounds used in the present invention and phosphate buffer are mixed to give injection.

### Formulation Example 8: Inhalations

The compounds used in the present invention and lactose are mixed and crushed finely to give inhalations.

### Formulation Example 9: Ointments

The compounds used in the present invention and petrolatum are mixed to give ointments.

### Formulation Example 10: Patches

The compounds used in the present invention and base such as adhesive plaster or the like are mixed to give patches.

### INDUSTRIAL APPLICABILITY

Based on the above test results, the compounds of the present invention can be a medicine useful as a therapeutic and/or prophylactic agent for symptoms and/or diseases induced by infection with mycobacteria.

1. A compound represented by formula (I): 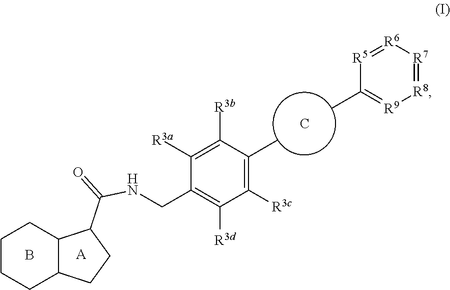
or its pharmaceutically acceptable salt,
wherein
a group represented by formula: 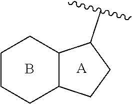
is a group represented by formula: 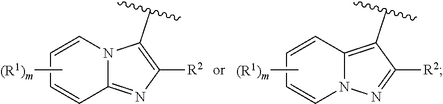
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted alkylsulfanyl, substituted or unsubstituted alkenylsulfanyl, substituted or unsubstituted alkynylsulfanyl, substituted or unsubstituted alkylsulfinyl, substituted or unsubstituted alkenylsulfinyl, substituted or unsubstituted alkynylsulfinyl, substituted or unsubstituted alkylsulfonyl, substituted or unsubstituted alkenylsulfonyl or substituted or unsubstituted alkynylsulfonyl;
m is 0, 1, 2, 3 or 4;
R² is a hydrogen atom, halogen, cyano, substituted or unsubstituted alkyl, substituted or unsubstituted alkenyl or substituted or unsubstituted alkynyl;
R^{3a}, R^{3b}, R^{3c} and R^{3d} are each independently hydrogen atom, halogen, hydroxy, cyano, substituted or unsubstituted alkyl, substituted or unsubstituted alkenyl or substituted or unsubstituted alkynyl, with the proviso that R^{3a}, R^{3b}, R^{3c} and R^{3d} are not simultaneously hydrogen atom;
ring C is represented as follows: 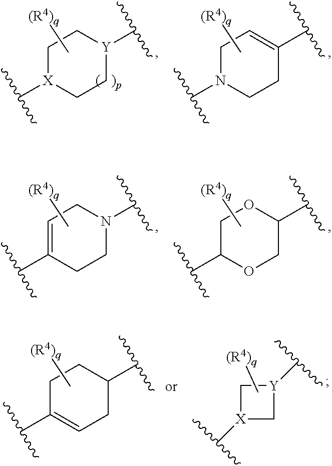
X is CH or N;
Y is CH or N;
R⁴ is each independently halogen, hydroxy, cyano, substituted or unsubstituted alkyl, substituted or unsubstituted alkenyl or substituted or unsubstituted alkynyl;
two R⁴ groups attached to adjacent carbon atoms may be taken together with the carbon atoms to which they are attached to form a substituted or unsubstituted 5- to 6-membered non-aromatic carbocycle or a substituted or unsubstituted 5- to 6-membered non-aromatic heterocycle;
two R⁴ groups attached to a same carbon atom may be taken together with the carbon atom to which they are attached to form a substituted or unsubstituted 3- to 6-membered non-aromatic carbocycle or a substituted or unsubstituted 3- to 6-membered non-aromatic heterocycle;
two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano or substituted or unsubstituted alkyl;
R^{4N} is each independently a hydrogen atom or substituted or unsubstituted alkyl;
p is 0 or 1;
q is 0, 1, 2, 3 or 4;
R⁵ is CR^{5C} or N;
R⁶ is CR^{6C} or N;
R⁷ is CR^{7C} or N;
R⁸ is CR^{5C} or N;
R⁹ is CR^{9C} or N;
with the proviso that R⁵, R⁶, R⁷, R⁸ and R⁹ are not simultaneously N;
R^{5C}, R^{6C}, R^{7C}, R^{8C} and R^{9C} are each independently hydrogen atom, halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, substituted or unsubstituted aromatic carbocyclyloxy, substituted or unsubstituted non-aromatic carbocyclyloxy, substituted or unsubstituted aromatic heterocyclyloxy, substituted or unsubstituted non-aromatic heterocyclyloxy or pentafluorothio;
with the proviso that compounds shown below are excluded:

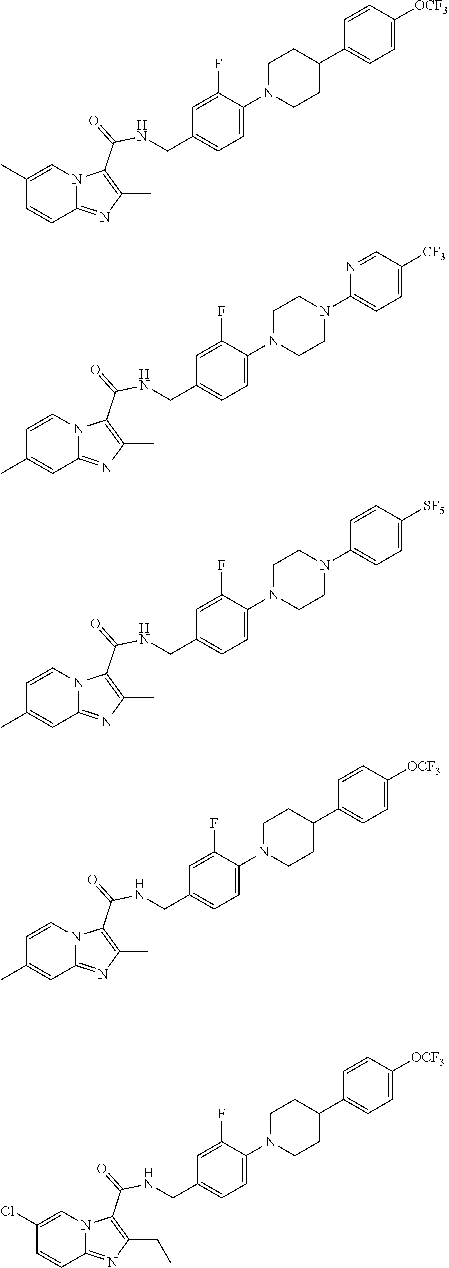

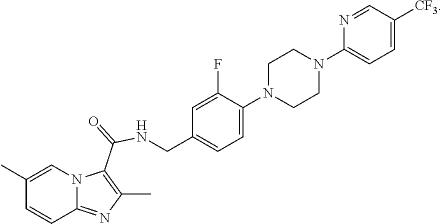 [SMILES: Cc1ccc2nc(C)c(C(=O)NCc3ccc(N4CCN(c5ccc(C(F)(F)F)cn5)CC4)c(F)c3)n2c1]

2. The compound according to claim 1 or its pharmaceutically acceptable salt, wherein
a group represented by formula: 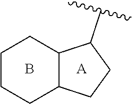
is a group represented by formula: 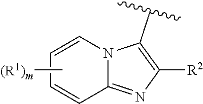
wherein each symbol is as defined in claim 1.

3. The compound according to claim 1 or its pharmaceutically acceptable salt, wherein
a group represented by formula: 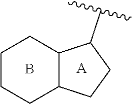
is a group represented by formula: 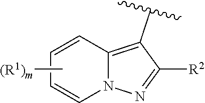
wherein each symbol is as defined in claim 1.

4. The compound according to claim 2 or its pharmaceutically acceptable salt, wherein R¹ is halogen or substituted or unsubstituted alkyl.

5. The compound according to claim 3 or its pharmaceutically acceptable salt, wherein R¹ is substituted or unsubstituted alkyloxy or substituted or unsubstituted alkyl.

6. The compound according to claim 1 or its pharmaceutically acceptable salt, wherein m is 1.

7. The compound according to claim 1 or its pharmaceutically acceptable salt, wherein R² is substituted or unsubstituted alkyl.

8. The compound according to claim 1 or its pharmaceutically acceptable salt, wherein R^{3b} is halogen.

9. The compound according to claim 1 or its pharmaceutically acceptable salt, wherein R^{3b} and R^{3c} are each independently halogen.

10. The compound according to claim 1 or its pharmaceutically acceptable salt, wherein R^{3a} is halogen.

11. The compound according to claim 1 or its pharmaceutically acceptable salt, wherein ring C is represented as follows: 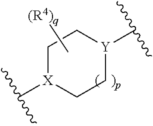
wherein each symbol is as defined in claim 1.

12. The compound according to claim 1 or its pharmaceutically acceptable salt, wherein p is 1.

13. The compound according to claim 1 or its pharmaceutically acceptable salt, wherein q is 0.

14. The compound according to claim 1 or its pharmaceutically acceptable salt, wherein q is 1.

15. The compound according to claim 1 or its pharmaceutically acceptable salt, wherein q is 2.

16. The compound according to claim 1 or its pharmaceutically acceptable salt, wherein ring C is represented as follows: 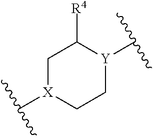
wherein each symbol is as defined in claim 1.

17. The compound according to claim 1 or its pharmaceutically acceptable salt, wherein ring C is represented as follows: 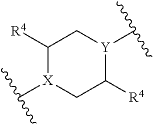
wherein each symbol is as defined in claim 1.

18. The compound according to claim 1 or its pharmaceutically acceptable salt, wherein X and Y are N.

19. The compound according to claim 1 or its pharmaceutically acceptable salt, wherein one of X and Y is N, and the other of X and Y is CH.

20. The compound according to claim 1, or its pharmaceutically acceptable salt, wherein R⁴ is each independently substituted or unsubstituted alkyl.

21. The compound according to claim 1 or its pharmaceutically acceptable salt, wherein R⁷ is CR^{7C}, and R^{7C} is substituted or unsubstituted alkyl or substituted or unsubstituted alkyloxy.

22. The compound according to claim 21 or its pharmaceutically acceptable salt, wherein R⁵, R⁶, R⁸ and R⁹ are CH.

23. The compound according to claim 21 or its pharmaceutically acceptable salt, wherein R⁵ is CR^{5C}, R^{5C} is halogen and R⁶, R⁸ and R⁹ are CH.

24. The compound according to claim 1 or its pharmaceutically acceptable salt, wherein the compound is selected from compounds (I-1-3), (I-1-25), (I-1-29), (I-1-38), (I-1-39), (I-1-42), (I-1-43), (I-1-45), (I-1-95) and (I-1-118).

25. The compound according to claim 1 or its pharmaceutically acceptable salt, wherein the compound is selected from compounds (I-1-144), (I-1-149) and (I-2-6).

26. A pharmaceutical composition comprising the compound according to claim 1 or its pharmaceutically acceptable salt.

27. The pharmaceutical composition according to claim 26, for the treatment and/or prevention of mycobacterial infection.

28. A method for preventing or treating mycobacterial infection, comprising administering the compound to a subject according to claim 1, or its pharmaceutically acceptable salt.

29. The compound according to claim 1, or its pharmaceutically acceptable salt, for the treatment and/or prevention of mycobacterial infection.
